(19) United States
(12) Patent Application Publication
(10) Pub. No.: US 2003/0165895 A1
(43) Pub. Date: Sep. 4, 2003

(54) Methods of detecting, diagnosing and treating cancer and identifying neoplastic progression

(76) Inventors: Bogdan Czerniak, Houston, TX (US); Dennis Johnston, Houston, TX (US)

Correspondence Address: Thomas M. Boyce, Fulbright & Jaworski L.L.P., Suite 2400, 600 Congress Avenue, Austin, TX, 78701, US

(21) Appl. No.: 10/167,241

(22) Filed: Jun. 11, 2002

Related U.S. Application Data
(60) Provisional application No. 60/297,813, filed on Jun. 12, 2001.

(51) Int. Cl. C12Q 1/68

(52) U.S. Cl. 435/6

(57) ABSTRACT

Disclosed are methods, compositions and apparatus useful in the detection, monitoring and treatment of the progression of neoplasia and preneoplastic conditions with special emphasis on the chromosomal changes related to the development and progression of urothelial neoplasia. Chromosomal changes, including LOH, at the disclosed loci demonstrate a statistically significant relation to the progression of disease state in urothelial neoplasia.

This application claims the benefit of priority to co-pending application serial No. 60/297,813, the entire disclosure of which is herein incorporated by reference.

The government owns rights in the present invention pursuant to grant numbers R29CA66723 and UO-1 CA85078 from the National Institutes of Health.

### BACKGROUND OF THE INVENTION

1. Field of the Invention

The present invention relates generally to the fields of cancer detection, diagnosis and prognosis. More particularly, it concerns methods, compositions and apparatus for the detection of neoplastic and preneoplastic cells associated with cancers, including urothelial tumors.

2. Description of Related Art

Cancer develops via multiple, cumulative steps, many of which precede the development of clinically and even microscopically recognizable disease. Conventional histologic mapping of invasive clinically evident cancer and adjacent tissues combined with clinical and epidemiological data conducted during the last 50 years provided compelling evidence for development of most epithelial cancers from precursor in situ conditions designated as dysplasia or carcinoma in situ. These conditions progress to invasive cancer by multiple cumulative molecular events, many of which are antecedent to the development of identifiable precursor lesions and occur in microscopically normal epithelium. Although, several models of human cancer progression from pre-malignant conditions has been proposed during the last decade the specific events leading to the development and progression of human neoplasia are largely unknown.

The analysis of genomic imbalances in human cancers can potentially guide us to those chromosomal regions that contain genes playing a role in tumor development and progression. Unfortunately, the analysis of such data is rarely informative as functional implications of the imbalances and consequently their pathogenetic significance are largely unknown. Moreover, it is unclear which of the imbalances are primary events relevant for disease progression and which are redundant hits dragged through the progression by mere cosegregation. In familial disorders including cancer predisposing syndromes a cosegregation of genetic hits with diseased phenotype identifies a predisposing locus and may guide subsequent positional cloning of a target gene. Unfortunately, the powerful concepts of genetic linkage analysis in pedigrees cannot be used in the vast majority of human cancers, which are sporadic disorders.

Recently, the first detailed look at the sequence of the human genome became available. Although still unfinished, the data provide great insight into the overall organization of the human genome. Additionally, the sequence presents a framework for the discovery of what may go wrong in human disease at molecular genetic level. The progress in mapping efforts has been accomplished by the gradual integration of recombination-based genetic maps with YAC contigs and EST radiation hybrid panels through the generation of BAC-based sequence-ready maps and finally to the ultimate map, the genome sequence itself These efforts are expected to make the future tasks of gene finding simpler and much less time consuming than current methods allow.

One obvious field of intensive research that requires a genome-wide approach has been the search for genes involved in the development and progression of common human cancers. Identification of those chromosomal loci and ultimately the target genes that play a role in the development of occult in-situ phases of neoplasia and their progression to clinically aggressive invasive cancer is of particular importance. Such information may provide clues for more specific studies on incipient phases of human carcinogenesis and facilitate future early cancer detection and as well as its ultimate prevention. In the past, genes involved in the development of human cancer were primarily identified by various positional cloning techniques from individual putative tumor suppressor gene loci defined by allelic loss or homozygous deletions and less frequently, from amplified chromosomal regions. More recently, the genome-wide search for both under- and over-expressed genes in various neoplastic disorders is being accomplished by various cDNA microarray technologies. Such an approach enables the identification of changes in expression patterns for hundreds or even thousands genes simultaneously, but cannot distinguish the primary events from secondary changes.

In searching genomic data with the goal of identifying genes involved in neoplastic initiation and progression, bladder carcinoma offers a useful model system because it develops by progression of microscopically recognizable in situ precursor conditions known as dysplasia and carcinoma in situ.

Urinary bladder cancer is the 5th most common cancer in the Western world and is responsible for approximately 3% of all cancer-related deaths. Tobacco smoking is correlated with half of all cases of bladder cancer. Another 25% of cases of bladder cancer are correlated with exposure to aromatic polycylic hydrocarbons or polychlorinated biphenyls in the environment. Approximately 55,000 new patients are diagnosed with bladder cancer annually in the United States, and approximately 15,000 of them die each year of the disease. The common urinary bladder tumors are derived from its transitional epithelium and comprise approximately 90% of bladder tumors. Transitional cell (urothelial) carcinoma (TCC) is the most common neoplasm of the urinary bladder in the Western world. Current pathogenetic concepts postulate that common urothelial neoplasms of the bladder arise via two distinct but somewhat overlapping pathways: papillary and nonpapillary. Approximately 80% of urothelial tumors of the bladder are superficially growing exophytic papillary lesions that may recur but usually do not invade and metastasize. They originate from hyperplastic urothelial changes. The remaining 20% of urothelial tumors are highly aggressive, solid, nonpapillary carcinomas with a strong propensity to invade and metastasize.

Bladder tumors are used as a common model of human cancer, which develops by progression of microscopically recognizable in situ precursor conditions, and are easily accessible by various minimally invasive or non-invasive techniques (Greenlee et al., 2000; Gazdar et al., 2001). The entire mucosal surface of the bladder can be examined by cystoscopy and biopsies with minimal risk for the patient and exfoliated urothelial cells can be repeatedly tested for various alterations in voided urine at no risk at all (Gazdar et al., 2001). Moreover, the simple anatomy and appropriates size of the bladder permit the histologic and genetic mapping studies of invasive cancer and preneoplastic lesions in the entire mucosa of cystectomy specimens.

The vast majority of invasive bladder cancers occur in patients without a prior history of papillary tumors and originate from clinically occult mild dysplasia (low-grade intraurothelial neoplasia) progressing to carcinoma in situ (high-grade intraurothelial neoplasia) and invasive cancer. The intraurothelial preneoplastic conditions progressing to invasive bladder cancer typically develop within the bladder epithelium as a primary lesion in a patient without any history of superficial papillary lesions. However, some patients who first present with low-grade, superficial papillary lesions may eventually develop intraurothelial neoplasia that progress first to carcinoma in situ and then to invasive cancer. In such instances, urothelial dysplasia and/or carcinoma in situ may develop in the adjacent urinary bladder epithelium or within the superficially growing papillary lesions.

It can be anticipated that tumors with such wide differences in morphology, growth pattern, and clinical behavior arise as a result of different molecular events. However, some overlapping molecular features may be present, especially in the early phases of neoplasia associated with establishment of an abnormal clone of urothelial cells within urinary bladder mucosa. The original dual-track concept of urinary bladder carcinogenesis, postulated approximately 20 years ago, was developed on the basis of clinicopathologic observations and whole-organ histologic mapping studies of cystectomy specimens. These early studies postulated that urothelial neoplasia progressed from precursor lesions such as low-grade dysplasia (low-grade intraurothelial neoplasia) to severe dysplasia and carcinoma in situ (high-grade intraurothelial neoplasia) and finally to invasive cancer. Furthermore, virtually every clinically evident lesion, such as superficial papillary tumors, was found to be associated with wide microscopically recognizable changes in the urinary bladder mucosa representing either hyperplasia or mild dysplasia. It is generally accepted now that invasive bladder cancer develops by the low-grade dysplasia-carcinoma in situ sequence via complex stepwise molecular events.

Bladder cancer is a highly accessible disease that is regularly monitored through a variety of noninvasive (urine) or minimally invasive (bladder barbotage, cystoscopy and biopsy) techniques. Approximately 80% of patients initially present with a superficial papillary lesion of low histologic grade. This type of tumor is typically treated by endoscopic resection. This technique is well tolerated, removes the cancer, and preserves bladder function. However, it is associated with a high rate of recurrence. Patients presenting with multifocal superficial papillary lesions have a risk of recurrence of 70% at 1 year. Patients with the most favorable presentation, i.e., a solitary superficial papillary lesion, still have a risk of recurrence approaching 50% at 4 years. Because of the high rate of recurrence, patients are routinely monitored by periodic cystoscopic examination, often as frequently as once every 3 months.

Approximately 15-20% of the patients who present with a low-grade superficial papillary lesion will eventually develop high-grade intraurothelial neoplasia somewhere else in the bladder that may progress to invasive high-grade bladder cancer. Although histologic assessment of the excised tumor (stage and grade) allows an estimate of the risk of progression and recurrence, it is still very imprecise. For example, approximately 30% of the patients whose tumors invade the lamina propria will experience progression to high-stage disease within 3 years. Conventional histopathological assessment of the excised neoplasm does not define which of these tumors are more likely to progress to high-stage disease and perhaps kill the patient. This variable natural history and the relative ease in obtaining specimens for sequential analysis make bladder cancer an excellent model for developing biomarkers.

Approximately 20% of patients present with high-grade invasive nonpapillary tumors, and they typically do not have a prior history of superficial papillary lesions. Despite the relatively easy access to the bladder both by direct vision (cystoscopy) and through analysis of exfoliated cells, conventional therapy including transurethral resection, intravesical chemotherapy, and immunotherapy frequently do not prevent tumor recurrences or late progression to high-stage and high-grade disease. Rare patients present with the de novo high-grade intraurothelial neoplasia (carcinoma in situ). More often, the development of high-grade intraurothelial neoplasia is observed clinically in a patient with a prior history of recurrent papillary urothelial tumors. Patients in whom high-grade intraurothelial neoplasia develop have a high risk of progression to invasive disease. In fact, most invasive urinary bladder tumors are of nonpapillary solid type that arise from carcinoma in situ.

While high-grade intraurothelial neoplasia (severe dysplasia/carcinoma in situ) classically presents as an area of redness, it may be visually indistinguishable from the remainder of the bladder and can be very difficult to detect. These patients are initially treated with intravesical bacille calmette guerin (BCG) and response is assessed by repeated urine cytologic examination and biopsy. Biomarkers that could improve the detection of this important tumor and especially its evolution from low-grade intraurothelial neoplasia could be used to identify patients whose disease is likely to progress to invasive cancer and are likely to require a more aggressive approach such as cystectomy.

High-grade lesions that are relatively superficial but invade the lamina propria (stage T1) are treated less aggressively but have a 30% risk of progression to muscle invasive disease (stage T2 or higher). These kinds of lesions are usually initially treated with local excision (transurethral resection) followed by intravesical BCG. A second resection is often performed, after completion of BCG treatment, to insure that the tumor is completely eradicated. Late recurrences, with the development of carcinoma in situ and early invasive T1 disease, are common and indicate potential for progression to high-stage disease.

The development of novel biomarkers that will provide early detection of tumors with the potentiality of progression to invasive disease would identify patients that require more aggressive therapy and/or new forms of intervention. Despite the initial effectiveness of intravesical BCG treatment, long-term recurrences of high-grade intraurothelial neoplasia are common, apparently owing in part to an underlying field change that may not necessarily be associated with the presence of microscopically and cytologically recognizable changes. Preliminary clinical evidence indicates that some vitamins and their analogues can lessen the propensity of low-grade intraurothelial neoplasia to become more aggressive or can diminish microscopically undetectable field changes. Current chemoprevention efforts are based on this concept, but they still lack information on the long-term effects and suitable markers for early monitoring of treatment effects, i.e., the eradication or persistence of genetically abnormal field changes in the bladder. Such markers can serve as an intermediate endpoint for chemoprevention and be extremely beneficial in assessing the effects of chemopreventive agents. They will help physicians monitor the disease once chemopreventive drugs become clinically available.

Finally, refractory superficial tumors and tumors that invade muscle are treated with cystectomy. Chemotherapy has been and is being administered in neoadjuvant (before cystectomy) and adjuvant (after cystectomy) strategies. The identification of biomarkers that can identify patients who are at risk of recurrence and development of distant metastases would significantly help in the choice of an appropriate neoadjuvant treatment that could preserve bladder function.

The ultimate identification and classification of urinary bladder neoplasia is accomplished by two pathological techniques, microscopic examination of tissue biopsies or transurethral resection specimens and urinary cytology. Tissue biopsies are accurate in classification of urothelial lesions that are identified by cystoscopic examination. They are less effective for evaluation of the presence of diffuse intraurothelial preneoplastic changes ranging from low- to high-grade intraurothelial neoplasia. The ineffectiveness of this approach is predominantly due to sampling error. Further, this technique cannot predict which of the intraurothelial preneoplastic conditions has a potentiality to progress to invasive disease nor which of the patients with superficial papillary lesions is more or less prone to develop the recurrence. Urine cytology alone has a low rate of detection of urinary bladder carcinoma; its accuracy varies from 50-70% depending on the number of specimens examined, the previous therapy, and the grade of the tumor. Cytologic interpretation is also frequently made difficult because of the low number of a typical or malignant cells present. Multiple auxiliary techniques have been used to improve the rate of detection and prediction of the biologic potential. The analysis of DNA ploidy both by image and flow cytology in bladder tumors helps to identify those grade 2 bladder lesions that are more likely to recur and progress. Virtually all high-grade nonpapillary and clinically aggressive lesions are aneuploid, while, superficial low-grade papillary lesions are often diploid. The analysis of DNA ploidy in voided urine specimens would also improve the rate of detection of urothelial neoplasia. In addition, various molecular techniques have been applied to identify genetic abnormalities in biopsies and voided urine specimens that range from the identification of mutated, transforming, and tumor suppressor genes, through identification of allelic losses in voided urine samples, to interphase genetics such as FISH studies.

The development of novel biomarkers for early detection and assessment of early signs of progression of urinary bladder neoplasia would be of utility in accomplishing the following goals.

1. Identify early changes as signs of clinically occult and even premicroscopic phases of urinary bladder neoplasia.

2. Develop rational treatment and chemoprevention strategies based on the assessment of individual patient risk of progression of intraurothelial neoplasia to invasive disease.

3. Assess success of chemoprevention effects for clinically occult disease, i.e., prevention of recurrence and progression.

4. Identify aggressive variants of bladder neoplasia whose progression to invasive disease is imminent and so justifies early, more aggressive intervention.

5. Illustrate the application of a genome-wide method of detecting the evolution of genetic changes underlying carcinogenesis.

### SUMMARY OF THE INVENTION

The instant application discloses a method of detecting the genetic changes in a subject related to the development and progression of cancers. Whole-organ histologic and genetic mapping are applied to early occult phases of human carcinogenesis.

In those sporadic human cancers that develop from microscopically recognizable pre-neoplastic conditions, the early predisposing events can be identified by the analysis of geographic relationship among genomic imbalances and precursor in situ conditions progressing to invasive disease. Such analysis can identify those hits that form plaques associated with growth advantage related to specific phases of neoplasia and are more likely to represent events driving the disease progression. In addition, the similarity of alterations such as loss of the same allele or the presence of identical molecular alterations in multiple samples corresponding to precursor conditions and invasive cancer identify their clonal relationship. Overall, the analysis of the relationship among the distribution of preneoplastic in situ conditions progressing to invasive cancer and genomic imbalances such as allelic loss and mutation provide data of major pathogenetic significance i.e. growth advantage and clonal relationship collectively referred to as clonal expansion.

The hits associated with clonal in situ expansion of abnormal cells involving large areas of mucosal membrane encompassing not only invasive cancer and precursor conditions but also some adjacent areas of microscopically normal epithelium represent early events associated with the development of incipient occult phases of neoplasia. On the opposite side of the spectrum are hits restricted to invasive carcinoma and adjacent areas of severe dysplasia and carcinoma in situ representing late events associated with the progression to invasive cancer. The superimposition of alteration patterns from all chromosomes in the entire mucosa of the affected organ provides more complete information on the sequence of events in disease progression.

One possible result of the present method is a genome-wide map of cancer progression from occult in situ precancerous conditions to clinically aggtressive invasive disease. One embodiment of the method integrates deletional chromosomal maps with physical maps and ultimately with the human genome sequence. The invention provides for the construction of an accurate map containign all known, proposed, and predicted genes mapping to chromosomal regions which are involved in clonal expansion of preneoplastic conditions, the progression to the state of invasive cancer, and the ultimate state of invasive cancer. The methods provide for analysis of the human genome sequences spanning target regions focussing on the content of repeat elements, their unique paleoontological and evolutionary features as well as the number and nature of genes mapping to these regions.

In one embodiment the invention comprises a method of generating a genome-wide map of cancer progression comprising the steps of (1) identifying significant associations between allele loss or mutation with other markers such as morphology, location (tissue distribution and geography within tissues), or other known neoplastic indicators, (2) performing a cluster analysis of allele loss or mutations identified in (1) with known genomic regions, e.g. chromosomes, or chromosome segments, (3) comparing the results of (1) and (2) to identify groups of allele loss or mutation with statistically significant association with the various phases of neoplasia. Further steps may optionally include the further analysis in order to identify subgroups of allele loss or mutation within other informative markers of neoplastic progression.

In a further, preferred embodiment, the methods comprise nearest neighbor analysis of the genomic location and significantly associated allele loss or mutation with other markers such as morphology, location (tissue distribution and geography within tissues), or other known neoplastic indicators. In further embodiments, the method further comprising overlapping groups of clonal allelic loss or mutation are overlain with geographical relationships of early and late phase neoplasia to indicate significant markers of allelic loss or mutation associated with such relationships.

In an additional embodiment, altered regions identified by the methods of the invention are associated with genomic markers present in the human genome. These associations may then be further converted to a purely physical map based upon the human genome sequence by correlating specific sequence markers available in the physical map (e.g. microsatellite markers, single nucleotide polymorphisms, etc.) with those identified to be significantly associated with the various neoplastic stages. Such specific sequence markers include all known, proposed and predicted gene sequences present in the human genome sequence, and which may also be correlated to other markers identified, such as mirosatelite markers, to produce an accurate map of all known, proposed, and predicted genes, as well as single nuleotide polymorphisms mapping to chromosomal regions identified as involved in the development and progression of the cancer so analyzed. Further details of functional and preferred embodiments may be found in the detailed description of the invention and in the exemplary studies provided.

The methods of the present invention may be referred to as whole-organ histologic and genetic mapping. In a specific example, these methods have been applied urothlial neoplasia. Bladder cancer was selected as close to an ideal model human tumor involving internal organs for studies of early events of carcinogenesis. The simple anatomy and appropriate size of the bladder permit histologic and genetic mapping studies of invasive cancer and in situ preneoplastic conditions in the entire mucosa of cystectomy specimens. A single cystectomy specimen can be divided into 30-60 mucosal samples each covering approximately 2 cm² of mucosal area and corresponding to normal urothelium, precursor intraurothelial conditions, and invasive carcinoma. The uroepithelial lining of the bladder is easily stripped from the underlying stromal tissue by simple mechanical scraping providing 99% pure urothelial cell suspensions. Such samples typically yield 5-10 μg of genomic DNA, ideal for studies of molecular genetic alterations in preneoplastic in situ lesions and sufficient for genome-wide PCR-based mapping studies. Moreover, the overall organization of the data permits the analysis of genetic alterations in relation to the disease progression by several powerful statistical algorithms such as nearest neighbor, binomial likelihood, and hierarchical clustering analyses.

The disclosure therefore indentifies chromosomal loci at which a loss of heterozygocity has been determined to be statistically related to either the development of urothelial neoplasia or the progression of the neoplastic phenotype from preneoplastic conditions through the development of invasive carcinoma. While the disclosed invention utilizes urothelial carcinoma as a model system, it is contemplated that the methods, and loci, disclosed are equally applicable to the detection of other neoplasia.

Thus, in a preferred embodiment of the instant invention, the disclosed methods are applicable to the detection of genetic changes relating to the development and progression of cancers. Such cancers would include brain cancer, liver cancer, spleen cancer, lymph node cancer, small intestine cancer, blood cell cancer, pancreatic cancer, colon cancer, stomach cancer, cervix cancer, breast cancer, endometrial cancer, prostate cancer, testicle cancer, ovarian cancer, skin cancer, head and neck cancer, esophageal cancer, oral tissue cancer, bone marrow cancer, lung cancer, cancers of the larynx, oral cavity, kidney and esophagus, bladder or urothelial cancer, and cervical cancer.

One embodiment of the invention relates a method of detecting a cell exhibiting a neoplastic or preneoplastic phenotype. This method comprises testing a sample containing cells for the presence of a loss of heterozygocity (LOH) at loci on one or more chromosomes. The chromosomes to be tested may be selected from the group consisting of: chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22. The identification of an LOH at one or more specific loci on these chromosomes is deemed indicative of a neoplastic or preneoplastic phenotype. In a preferred embodiment, the neoplastic phenotype is an urothelial neoplasia.

In a preferred embodiment, the disclosed loci are utilized in the construction of probes which may be assembled in DNA arrays and/or on DNA chips for the detection of the chromosomal changes related to the development of a preneoplastic or neoplastic phenotype or to monitor the progression of genetic changes during cancers. In one embodiment, the DNA array or DNA chip would comprise DNA probes corresponding to loci on at least three chromosomes. The chromosomes to be assayed would be selected from the group including chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22. Detection of an LOH at one or more specific loci, as disclosed herein, is indicative of a neoplastic or preneoplastic phenotype.

A further embodiment would involve the selection probes specific for one or more chromosomes selected from the group including chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22. In a preferred embodiment, either of these proposed arrays would be useful in the specific detection of urothelial neoplasia.

In another embodiment, the detection of the disclosed genetic alterations permits the determination of specific stages within the progression of the neoplastic phenotype. It is envisioned that the instant invention encompasses a method of detecting urothelial neoplasia. This method would comprise obtaining a urine sample or bladder tissue sample, isolating bladder cells from the sample and testing the bladder cells for allelic loss at loci associated with the development of urothelial neoplasia. The loci to be assayed may be selected from the group consisting of D1S243, D1S1608, D1S548, D1S198, D1S221, APOA2, TPo, D2S1240, D2S378, D2S114, D2S294, D2S159, D3S1298, D3S1278, D3S1303, D3S1541, ACPP, D3S1512, D3S1246, D3S1754, D3S1262 and D3S1661 D4S405, D4S828, D4S1548, D4S1597, D4S1607, D4S408, D5S428, APCII, D5S346, D5S421, MCC, D5S659, D5S404, D5S2055, D5S818, IRF1, CFS1R, D5S1465, EDN1, D6S251, D6S262, D6S290, D6S1027, D7S526, D8S136, D8S133, D8S137, D8S259, ANKI, D8S285, D8S553, D9S286, D9S156, D9S304, D9S273, D9S166, D9S252, D9S287, D9S180, D9S66, D10S1214, D10S213, D10S606, D10S215, D10S1242, D10S190, D10S217, D11S922, D11S569, D11S2368, D11S1301, D11S937, D11S931, D11S897, D11S924, D11S1284, D11S933, D11S910, D12S397, D13S221, D13S171, D13S291, RB1, D13S164, D13S268, D13S271, D13S154, D14S290, D14S68, D15S207, D15S107, D16S513, D16S500, D16S541, D16S415, D16S512, D16S505, D16S520, D17S578, D17S849, TP53, D17S960, D17S786, D17S799, D17S947, D17S579, D17S933, D17S932, D17S934, D17S943, D17S807, D17S784, D18S452, D18S66, D18S68, D19S406, D19S714, D19S225 D22S264, D22S446, D22S280 and D22S423.

An embodiment of the invention involves the detection of occult preclinical or premicroscopic stages of urothelial neoplasia by LOH assay, wherein the assayed loci are selected from the group including: D1S243, D1S1608, D1S548, D1S198, D1S221, APOA2, TPO, D2S1240, D2S378, D2S114, D2S294, D2S159, D3S1298, D3S1278, D3S1303, D3S1541, ACPP, D3S1512, D3S1246, D3S1754, D3S1262 and D3S1661 D4S405, D4S828, D4S1548, D4S1597, D4S1607, D4S408, D5S428, APCII, D5S346, D5S421, MCC, D5S659, D5S404, D5S2055, D5S818, IRF1, CFS1R, D5S1465, EDN1, D6S251, D6S262, D6S290, D6S1027, D7S526, D8S136, D8S133, D8S137, D8S259, ANKI, D8S285, D8S553, D9S286, D9S156, D9S304, D9S273, D9S166, D9S252, D9S287, D9S180, D9S66, D10S1214, D10S213, D10S606, D10S215, D10S1242, D10S190, D10S217, D11S922, D11S569, D11S2368, D11S1301, D11S937, D11S931, D11S897, D11S924, D11S1284, D11S933, D11S910, D12S397, D13S221, D13S171, D13S291, RB1, D13S164, D13S268, D13S271, D13S154, D14S290, D14S68, D15S207, D15S107, D16S513, D16S500, D16S541, D16S415, D16S512, D16S505, D16S520, D17S578, D17S849, TP53, D17S960, D17S786, D17S799, D17S947, D17S579, D17S933, D17S932, D17S934, D17S943, D17S807, D17S784, D18S452, D18S66, D18S68, D19S406, D19S714, D19S225 D22S264, D22S446, D22S280 and D22S423.

It is envisioned that cells to be sampled may be obtained from a variety of sources within a host. In certain embodiments of the instant invention, cells may be obtained from voided urine or by branchial lavage. In other embodiments, the cells may be obtained from bladder tissue samples.

It is further envisioned that a variety of techniques may be used to detect the genetic changes that are indicative of the development of a neoplastic or preneoplastic phenotype. In a preferred embodiment of the instant invention, such a change is detectable by the use of a gene chip or DNA array. In further embodiments, changes are detectable by fluorescent in situ hybridization (FISH), southern blotting, PCR analysis, or RFLP analysis.

For the purpose of the instant invention, a variety of loci may be screened as indicative of the development of a neoplastic or preneoplastic phenotype. In certain embodiments, loci on chromosome 1 would consist of D1S243, DlS1608, D1S548, D1S198, Dl S221 and APOA2; loci on chromosome 2 would consist of TPO, D2S1240, D2S378, D2S114, D2S294 and D2S159; loci on chromosome 3 would consist of D3S1298, D3S1278, D3S1303, D3S1541, ACPP, D3S1512, D3S1246, D3S1754, D3S1262 and D3S1661; loci on chromosome 4 would consist of D4S405, D4S828, D4S1548, D4S1597, D4S1607 and D4S408; loci on chromosome 5 would consist of D5S428, APCII, D5S346, D5S421, MCC, D5S659, D5S404, D5S2055, D5S818, IRF1, CFS1R and D5S1465; loci on chromosome 6 would consist of EDN1, D6S251, D6S262, D6S290 and D6S1027; loci on chromosome 7 would consist of D7S526; loci on chromosome 8 would consist of D8S136, D8S133, D8S137, D8S259, ANKI, D8S285 and D8S553; loci on chromosome 9 would consist of D9S286, D9S156, D9S304, D9S273, D9S166, D9S252, D9S287, D9S180 and D9S66; loci on chromosome 10 would consist of D10S1214, D10S213, D10S606, D10S215, D10S1242, D10S190 and D10S217; loci on chromosome 11 would consist of D11S922, D11S569, D11S2368, D11S1301, D11S937, D11S931, D11S897, D11S924, D11S1284, D11S933 and D11S910; loci on chromosome 12 would consist of D12S397; loci on chromosome 13 would consist of D13S221, D13S171, D13S291, RB1, D13S164, D13S268, D13S271 and D13S154; loci on chromosome 14 would consist of D14S290 and D14S68; loci on chromosome 15 would consist of D15S207 and D15S107; loci on chromosome 16 would consist of D16S513, D16S500, D16S541, D16S415, D16S512, D16S505 and D16S520; loci on chromosome 17 would consist of D17S578, D17S849, TP53, D17S960, D17S786, D17S799, D17S947, D17S579, D17S933, D17S932, D17S934, D17S943, D17S807 and D17S784; loci on chromosome 18 would consist of D18S452, D18S66 and D18S68; loci on chromosome 19 would consist of D19S406, D19S714 and D19S225; and loci on chromosome 22 would consist of D22S264, D22S446, D22S280 and D22S423.

For the purpose of the instant invention, urothelial neoplasia comprises the progression of the neoplastic state from preneoplastic conditions to invasive cancer within the urinary bladder and surrounding tissue.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Genetic model of human urothelial carcinogenesis. The map was assembled on the basis of whole-organ histologic and genetic mapping of chromosomes 1-22. Outer circle represents chromosomal vectors aligned clockwise from p to q arms, with positions of altered markers exhibiting LOH. All the markers are positioned on the vectors according to the human genome database (version Mar. 14, 1996). The innermost concentric circles represent major phases of development and progression of urothelial neoplasia from normal urothelium (NU) through low-grade intraurothelial neoplasia (LGIN) and high-grade intraurothelial neoplasia (HGIN) to transitional cell carcinoma (TCC). Solid circles (●) denote statistically significant LOH of the markers defined by the LOD score analysis. Open circles (∘) identify LOH without statistically significant association to a given stage of neoplasia. The positions of open or solid circles on appropriate concentric circles relate the alterations to a given phase of neoplasia. Only markers with LOH are positioned on the chromosomal vectors. Solid bars on outer brackets represent clusters of markers with significant LOH and denote location of putative tumor suppressor genes involved in urothelial neoplasia. The distances of markers on chromosomal vectors and the solid bars depicting minimal deleted regions were adjusted to fit the circle and are not drawn to scale. More precise localization of these regions can be obtained from individual chromosomal vectors

FIG. 2. Assembly of a three-dimensional display of LOH on five tested chromosomes in a single cystectomy specimen with invasive TCC. The vertical axis represents vectors with positions of hypervariable markers and their chromosomal location. Only markers with LOH are shown. The shaded blocks represent areas of urinary bladder mucosa with LOH as they relate to progression of neoplasia represented by a histologic map of cystectomy specimen with invasive bladder cancer and adjacent precursor conditions in the background. In addition to an area of invasive cancer, there are two separate foci of non-invasive papillary TCC. The histologic map code is: NU, normal urothelium; MD, mild dysplasia; MdD, moderate dysplasia; SD, severe dysplasia; CIS, carcinoma in situ; TCC, transitional cell carcinoma. For the purpose of statistical analyses precursor conditions were grouped as follows: MD, and MdD, low-grade intraurothelial neoplasia (LGIN); MdD and CIS, high-grade intraurothelial neoplasia (HGIN). Note that there is wide involvement of almost the entire urinary bladder mucosa by LOH in loci D17S786 and D8S553 representing earliest hits in the evolution of urothelial neoplasia detectable by this approach. An accumulation of allelic losses on chromosome 9 in two foci of noninvasive papillary TCCs is present, but not in the areas of invasive TCC. Scattered, apparently separate foci of allelic losses occured in areas of urinary bladder mucosa with wide field type allelic losses in loci D17S786 and D8S553.

FIG. 3. Testing of frequency of LOH in voided urine samples and bladder tumor samples on patients with urinary bladder cancer in target minimally deleted regions on chromosomes 3, 9, and 13. A) Summary of allelic loss of chromosome 3 tested with 17 hypervariable markers in 22 voided urine and 32 urinary bladder tumor samples. The list of 17 tested markers and their chromosomal locations are provided at the top. The allelic losses are related to clinicopathologic parameters such as growth pattern, histologic grade, stage of tumor and follow up data. It is evident that allelic losses in the ACPP region form a clearly defined locus. Allelic losses and occasional shortening or expansion of repetitive sequences of the hypervariable markers in the remaining tested regions of chromosome 3 seem to represent random events without clustering in clearly defied loci. B) Summary of allelic loss of chromosome 9 tested with 20 hypervariable markers in 26 samples. The list of 20 tested markers and their chromosomal locations are provided at the top. The allelic losses are related to clinicopathologic parameters such as growth pattern, histologic grade, stage of tumor and follow up data. C) Summary of allelic loss of chromosome 13 tested with 12 hypervariable markers. The list of 12 tested markers and their chromosomal locations are provided at the top. The allelic losses are related to clinicopathologic parameters such as growth pattern, histologic grade, stage of tumor and follow up data.

FIG. 4. Summary of data on allelic losses on chromosome 3. The Genenthon chromosome vector with a list of tested markers and their distances in centimorgans (cM). Additional markers delineated by solid bars on the left were added to the vectors. All the markers are positioned according to the Human Genome Database (version Mar. 14, 1996). The data on allelic losses revealed by superimposed histologic and genetic mapping are summarized in the middle column designated SHGM. Individual rows numbered 1-8 designate the results in individual cystectomy specimens. Open circles (∘) indicate markers without evidence of LOH. Solid circles (●) denote markers with LOH. Open circles with slash (ø) indicate non-informative marker. An asterisk (*) on the right side of the marker indicates a statistically significant association between an altered marker and urothelial neoplasia as established by LOD score. Thin vertical lines on the left side of the chromosomal diagram designated putative locations of the marker on chromosomal regions. The chromosomal locations are provided only for markers with LOH. Solid bars on the left of the chromosomal vector identify the minimal deleted regions. These regions are defined by flanking markers and the predicted size of the deleted segment in cM. In general, the diagram shows scattered regions of LOH on both arms of chromosome 3. The markers exhibiting LOH with statistically significant LOD scores clustered in two distinct regions that may contain putative tumor suppressor genes involved in the development and progression of urinary bladder cancer. The regions defined by the nearest markers flanking the microsatellite exhibiting LOH with significant LOD scores were: D3S1277-D3S1100, (p21) and D3S1541-D3S1512, q(21-25). Larger areas of deletion involving q21-25 and q26-27 regions are seen in a single case of cystectomy specimens (map 5). The markers and deleted regions implicated in the development and progression of neoplasia are shown here without designation of particular phases of urothelial neoplasia. Their relationship to the development of various phases of intraurothelial neoplasia can be obtained from the LOD score table shown in the bottom panel and from the genetic model shown in FIG. 2.

Cumulated LOD scores for allelic losses of chromosome 3 markers were calculated and analyzed for different phases of urothelial changes ranging from NU, normal urothelium; LGIN, low-grade intraurothelial neoplasia; HGIN, high-grade intraurothelial neoplasia and TCC, transitional cell carcinoma. To simplify the table, only stringency level 1 calculations are shown. The pattern of LOD scores ≧3 at θ=0.01 or 0.99 and LOD scores <3 at θ=0.5 for the same marker is significant. The strongest association between an altered marker and neoplasia is when a LOD score is ≧3 at θ=0.9 and 0.5 and <3 at θ=0.01. Note that the significant patterns of LOD scores typically parallel lower values of Tₘₐₓ. Note that allelic losses in the ACPP marker show statistically significant LOD score with the morphologically normal urothelium and precede the development of microscopically recognizable changes such as LGIN. Allelic losses of this marker retain the statistically significant score through all subsequent stages of urothelial neoplasia ranging from LGIN to TCC. The large segments of the flanking areas of the q arm involving q21-25 and q26-28 regions developed statistically significant LOD scores in progression to invasive disease. Similarly, the allelic loss of the marker D3S1298 exhibits statistically significant LOD score in association with the development of invasive TCC.

FIG. 5. Summary of whole-organ histologic and genetic mapping of deleted regions on chromosome 1 involved in progression of human urinary bladder neoplasia from preneoplastic intraurothelial lesion to invasive cancer.

FIG. 6. Summary of whole-organ histologic and genetic mapping of deleted regions on chromosome 2 involved in progression of human urinary bladder neoplasia from preneoplastic intraurothelial lesion to invasive cancer.

FIG. 7. Summary of whole-organ histologic and genetic mapping of deleted regions on chromosome 4 involved in progression of human urinary bladder neoplasia from preneoplastic intraurothelial lesion to invasive cancer.

FIG. 8. Summary of whole-organ histologic and genetic mapping of deleted regions on chromosome 5 involved in progression of human urinary bladder neoplasia from preneoplastic intraurothelial lesion to invasive cancer. A. Genetic map of chromosome 5 with a list of tested markers and their distances. Chromosomal locations are provided for altered markers only. All the markers were positioned on the map according to the Cooperative Human Linkage Center map (version 4.0). Asterisks on the right side of the markers indicate statistically significant association between an altered marker and urothelial neoplasia as established by LOD scores. Bars on the left side of the chromosomal vector identify the deleted regions associated with the development and progression of urothelial neoplasia. The regions of allelic losses defined by the nearest nonaltered flanking markers and their predicted size in cM are as follows: 5q13.3-q22 (D5S424-D5S656, 38.8 cM), 5q22-q31.1 (D5S656-D5S808, 19.2 cM), 5q31.1-q32 (D5S816-SPARC, 11.5 cM) and 5q34 (GABRA1-D5S415, 6.4 cM). The relationship of markers with LOH to various phases of neoplasia is provided in the LOD score table shown in B. (cM, centimorgans; WOHGM, whole-organ histologic and genetic mapping of individual cystectomy specimens consecutively numbered 1 through 5. ◯—nonaltered marker, ●—markers with LOH, and Ø—noninformative marker). B. Summary of binomial maximum likelihood analysis testing the relationship among LOH in individual chromosome 5 loci and progression of urothelial neoplasia from in situ precursor conditions to invasive TCC. Cumulative LOD scores for markers with LOH were calculated at variable θ=(0.01, 0.5, and 0.99) and tested against Tmax. The significance of allelic losses in individual loci was analyzed for normal urothelium (NU); low-grade intraurothelial neoplasia (LGIN); high-grade intraurothelial neoplasia (HGIN) and transitional cell carcinoma (TCC). To simplify the data, stringency 1 calculations are presented only. The patterns of significant LOD scores are as described below. Note that significant patterns of LOD scores typically parallel the high T max values. (◯—LOD score <3; ●—LOD score ≧3).

FIG. 9. Summary of whole-organ histologic and genetic mapping of deleted regions on chromosome 6 involved in progression of human urinary bladder neoplasia from preneoplastic intraurothelial lesion to invasive cancer.

FIG. 10. Summary of whole-organ histologic and genetic mapping of deleted regions on chromosome 7 involved in progression of human urinary bladder neoplasia from preneoplastic intraurothelial lesion to invasive cancer.

FIG. 11. Summary of whole-organ histologic and genetic mapping of deleted regions on chromosome 8 involved in progression of human urinary bladder neoplasia from preneoplastic intraurothelial lesion to invasive cancer.

FIG. 12. Summary of whole-organ histologic and genetic mapping of deleted regions on chromosome 9 involved in progression of human urinary bladder neoplasia from preneoplastic intraurothelial lesion to invasive cancer.

FIG. 13. Summary of whole-organ histologic and genetic mapping of deleted regions on chromosome 10 involved in progression of human urinary bladder neoplasia from preneoplastic intraurothelial lesion to invasive cancer.

FIG. 14. Summary of whole-organ histologic and genetic mapping of deleted regions on chromosome 11 involved in progression of human urinary bladder neoplasia from preneoplastic intraurothelial lesion to invasive cancer.

FIG. 15. Summary of whole-organ histologic and genetic mapping of deleted regions on chromosome 12 involved in progression of human urinary bladder neoplasia from preneoplastic intraurothelial lesion to invasive cancer.

FIG. 16. Summary of whole-organ histologic and genetic mapping of deleted regions on chromosome 13 involved in progression of human urinary bladder neoplasia from preneoplastic intraurothelial lesion to invasive cancer.

FIG. 17. Summary of whole-organ histologic and genetic mapping of deleted regions on chromosome 14 involved in progression of human urinary bladder neoplasia from preneoplastic intraurothelial lesion to invasive cancer.

FIG. 18. Summary of whole-organ histologic and genetic mapping of deleted regions on chromosome 15 involved in progression of human urinary bladder neoplasia from preneoplastic intraurothelial lesion to invasive cancer.

FIG. 19. Summary of whole-organ histologic and genetic mapping of deleted regions on chromosome 16 involved in progression of bladder neoplasia from intraurothelial precursor conditions to invasive cancer. (A) Map of chromosome 16 with a list of tested markers and their positions according to the Genethon database, version March, 1996. Asterisks on the right side of the markers indicate a statistically significant association between an altered marker and urothelial neoplasia. Bars on the left side of the chromosomal vector designate deleted regions defined by their flanking markers and a size in cM as follows:

p13.3(D16S418-D16S406, 1.2cM), p13.1(D16S748-D16S287, 12.9cM), q12.1(D16S409-D16S514, 24.0cM), q22.1(D16S496-D16S515, 5.4cM), q24 (D16S507-D16S511, 5.9CM) and q24(D16S402-D16S413, 17.4cM). The relationship of LOH in individual markers to various phases of urothelial neoplasia was tested by binomial maximum likelihood analysis and is summarized in the LOD score table shown in B. (cM, centimorgans; WOHGM, whole organ histologic and genetic mapping of individual cystectomy specimens consecutively numbered 1 through 5. ◯ nonaltered marker; ●, altered marker; 100 , noninformative marker) (B) Binomial maximum likelihood analysis testing the relationship among LOH of individual chromosome 16 markers and progression of bladder neoplasia from intraurothelial precursor conditions to invasive cancer. Cumulative LOD scores for chromosome 16 markers with LOH were calculated at variable θ=(0.01, 0.5 and 0.99) for normal urothelium (NU); low-grade intraurothelial neoplasia (LGIN); high-grade intraurothelial neoplasia (HGIN); and transitional cell carcinoma (TCC). To simplify graphical presentation only stringency 1 calculations are provided. The patterns of statistically significant LOD scores are as described below. Note that significant patterns of LOD scores typically correspond to high Tₘₐₓ values. (◯, LOD score <3;, LOD score ≧3).

FIG. 20. Summary of whole-organ histologic and genetic mapping of deleted regions on chromosome 17 involved in progression of human urinary bladder neoplasia from preneoplastic intraurothelial lesion to invasive cancer.

FIG. 21. Summary of whole-organ histologic and genetic mapping of deleted regions on chromosome 18 involved in progression of human urinary bladder neoplasia from preneoplastic intraurothelial lesion to invasive cancer.

FIG. 22. Summary of whole-organ histologic and genetic mapping of deleted regions on chromosome 19 involved in progression of human urinary bladder neoplasia from preneoplastic intraurothelial lesion to invasive cancer.

FIG. 23. Summary of whole-organ histologic and genetic mapping of deleted regions on chromosome 20 involved in progression of human urinary bladder neoplasia from preneoplastic intraurothelial lesion to invasive cancer.

FIG. 24. Summary of whole-organ histologic and genetic mapping of deleted regions on chromosome 21 involved in progression of human urinary bladder neoplasia from preneoplastic intraurothelial lesion to invasive cancer.

FIG. 25. Summary of whole-organ histologic and genetic mapping of deleted regions on chromosome 22 involved in progression of human urinary bladder neoplasia from preneoplastic intraurothelial lesion to invasive cancer.

FIG. 26. Summary of target loci on the q arm of chromosome 3 involved in urinary bladder cancer. The YAC contig maps of the minimally deleted (q21-22) and amplified (q24-26) loci as well as the examples of our dual labeling FISH studies with YAC825b3 (top panel) and BAC 522C10 (bottom panel) are shown. YAC825b3: (A) A control test with chromosome 3 from normal human lymphocytes. (B) A control test with normal human lymphocytes. Two YAC and two centromeric signals are present. (C) Examples of allelic losses documented with YAC825b3. One cell shows only one YAC probe signal with two centromeric CEP3 signals. The upper cell shows chromosome 3 polysomy (3 centromeric signals and only two YAC probe signals). BAC522C10: (A) A control test with chromosome 3 from normal lymphocytes. (B) A control test with normal human lymphocytes. Two BAC and two centromeric signals are present. (C) Examples of allelic loss. Two centromeric signals and only one signal with BAC probe are present. (D) An example of homozygous deletion. Two centromeric signals are present but no BAC signal could be documented.

FIG. 27 Assembly of superimposed histologic and genetic maps. A) Examples showing consistent LOH of the same allele in multiple mucosal samples of the same cystectomy specimen. Marker D9S273 shows LOH in multiple samples corresponding to TCC (samples 39-41) and involving areas of urinary bladder mucosa exhibiting changes consistent with LGIN and HGIN (samples 31, 33, and 36), as well as an area with microscopically normal urothelium (sample 19). Marker D9S1124 shows LOH in four samples. Samples 39 and 40 corresponded to invasive TCC. Samples 32 and 38 corresponded to HGIN. Marker D9S424 shows LOH only in an area corresponding to invasive TCC (sample 34). In summary, marker D9S273 shows LOH in invasive TCC and precursor in situ conditions (LGIN and HGIN) as well as in an area of microscopically normal urothelium, indicating that LOH in this locus is an early event. Marker D9S1124 developed LOH in HGIN that progressed to invasive TCC and is a relatively late event associated with the development of high-grade urothelial dysplasia and/or carcinoma in situ. LOH of marker D9S424 is a late event associated with the development of invasion. Sample #1 in all the panels represents the allelic pattern of the marker from peripheral blood of the same patient and serves as a control. The presence of LOH in all samples was confirmed by densitometry and is expressed as O.D. ratio below each sample. O.D. ≦0.5 is indicative of LOH. Solid bars below panels denote samples with LOH. B) Examples of superimposed histologic and genetic maps of three cystectomy specimens. Marker D11S1301 (left panel) shows scattered foci of LOH. Marker D4S1548 (middle panel) shows a plaque-like LOH involving almost the entire urinary bladder mucosa. Marker D17S849 (right panel) shows LOH restricted to invasive TCC only. Open boxes delineated by black lines indicate areas of urinary bladder mucosa with LOH in a given locus. The background shadowed area represents a histologic map of cystectomy specimen depicting distribution of various intraurothelial precursor conditions and TCC. Histologic map code: (NU) normal urothelium; (MD) mild dysplasia; (MdD) moderate dysplasia; (SD) severe dysplasia; (CIS) carcinoma in situ; (TCC) transitional cell carcinoma.

FIG. 28. Summary of physical map analysis spanning the deleted regions of chromosome 1.

FIG. 29. Summary of physical map analysis spanning the deleted regions of chromosome 2.

FIG. 30. Summary of physical map analysis spanning the deleted regions of chromosome 3.

FIG. 31. Summary of physical map analysis spanning the deleted regions of chromosome 4.

FIG. 32. Summary of physical map analysis spanning the deleted regions of chromosome 5. Original markers and substitutes for markers with LOH based on the closest proximity were placed on the Genethon map and were repositioned on the GB4 radiation hybrid panel-based physical map. The new positions for the Genethon markers with LOH as well as flanking markers on the GB4 map were identified by electronic PCR search of BAC contigs. In addition, multiple alternative markers based on their proximity to markers with LOH were identified and added to the map. The original Genethon markers with LOH are shown in gray. All other substitute and flanking markers are printed in black. In addition, average EST density for regions flanked by individual markers placed on GB4 map and a list of 138 known genes mapping to the target regions are shown. To simplify the diagram, contig data used for this analysis are not provided. More complete data with alternative positions of the genes can be obtained from http://www.mdanderson.org/bladdergenomicmaps. (cM-centimorgan, cR-centiray)

FIG. 33. Summary of physical map analysis spanning the deleted regions of chromosome 7.

FIG. 34. Summary of physical map analysis spanning the deleted regions of chromosome 10.

FIG. 35. Summary of physical map analysis spanning the deleted regions of chromosome 13.

FIG. 36. Summary of physical map analysis spanning the deleted regions of chromosome 14.

FIG. 37. Summary of physical map analysis spanning the deleted regions of chromosome 15.

FIG. 38. Summary of physical map and sequence database analysis spanning the deleted regions of chromosome 16. The Genethon positions of the markers defining the deleted regions were related to the GB4 radiation hybrid panel-based physical map. The new positions for the Genethon markers with LOH as well as flanking markers on the GB4 map were identified by electronic PCR search of BAC contigs. In addition, multiple alternative markers based on their proximity to markers with LOH were identified and added to the map. The nearest substitute markers are often located within the same BAC clone as original Genethon markers used for LOH studies. Consequently some of the original Genethon and substitute markers have the same position on the GB4 map. The original Genethon markers with LOH are shown in red. All other substitute and flanking markers are printed in black. An average EST density is provided for regions flanked by individual markers. The list of known genes within the target regions and their positions on the GB4 map is also shown. To simplify the diagram, only the first position of a known gene sequence on the GB4 map is shown. More complete data with contigs information and alternative positions of the genes can be obtained from http://www.mdanderson.org/BladderGenomicMaps/

FIG. 39. Summary of physical map analysis spanning the deleted regions of chromosome 17.

FIG. 40. Summary of physical map analysis spanning the deleted regions of chromosome 18.

FIG. 41. Summary of physical map analysis spanning the deleted regions of chromosome 19.

FIG. 42. Summary of physical map analysis spanning the deleted regions of chromosome 22.

FIG. 43. Assembly of whole-organ histologic and genetic maps. A. An example of marker tested on multiple mucosal samples from the cystectomy specimen (map 5). Marker IRF1 shows LOH in samples corresponding to TCC (samples 4, 15 and 16) as well as ones exhibiting changes consistent with LGIN (samples 13, 19, 22 and 27) and HGIN (samples 2, 3, 5-12, 14, 17, 18, 20,21, 23, 24, 26, 28-35). Sample #1 represents allelic patterns of the same marker from peripheral blood of the same patient and serves as control. The presence of LOH in all samples was confirmed by densitometry and is expressed as O.D. ratio below each sample in both panels. O.D.<0.5 is indicative of LOH. B. Example of chromosome 5 allelic losses in a single cystectomy specimen (map 5) with invasive non-papillary urothelial carcinoma assembled by nearest neighbor analysis. The vertical axis represents a chromosome 5 map with positions of markers and their chromosomal locations. Only altered markers are shown. The shaded blocks represent areas of urinary bladder mucosa with LOH as they relate to progression of neoplasia presented by a histologic map of cystectomy in the background. Note that several markers including IRF1 show LOH in a form of a plaque involving a large area of urinary bladder mucosa. Code for a histologic map is shown in B. C. Examples of LOH distributions superimposed on a histologic map of cystectomy specimen (map 5). Markers D5S346 and IRF1 show a plaque-like LOH involving almost the entire urinary bladder mucosa. Marker D5S1465 shows LOH involving smaller area of urinary bladder mucosa located within a larger plaque of LOH which involved markers D5S346 and IRF1. Open boxes delineated by lines indicate areas of urinary bladder mucosa with alterations in a given locus. The background-shadowed area represents a histologic map of cystectomy specimen depicting distribution of various intraurothelial precursor conditions and TCC. Histologic map code: (NU) normal urothelium; (MD) mild dysplasia; (MdD) moderate dysplasia; (SD) severe dysplasia; (CIS) carcinoma in situ; (TCC) invasive transitional cell carcinoma.

FIG. 44. Assembly of whole-organ histologic and genetic maps. (A) Example of a marker D16S541 tested on multiple mucosal samples from the same cystectomy specimen (map 4). Sample 1 represents allelic patterns of the marker from peripheral blood lymphocytes of the same patient and serves as control. Marker D16S541 shows LOH in samples corresponding to microscopically normal urothelium (samples 2-5, 7, and 8), LGIN (samples 9-11, and 19) and invasive TCC (sample 24). The presence of LOH in all samples was confirmed by densitometry and is expressed as O.D. ratio below each sample. O.D. ratio ≦0.5 was considered indicative of LOH. (B) Example of chromosome 16 allelic losses in a single cystectomy specimen with invasive TCC assembled by nearest neighbor analysis. The vertical axis represents a chromosome 16 vector with positions of markers and their chromosomal locations. Only markers with LOH are shown. The shaded blocks represent areas of urinary bladder mucosa with LOH as they relate to progression of neoplasia presented by a histologic map of cystectomy in the background. The code for histologic map is as shown in C. (C) Example of whole—organ histologic and genetic map of a cystectomy specimen showing distribution of LOH in three markers on chromosome 16. Markers D16S505 and D16S520 show an almost identical overlapping plaque-like LOH involving a large area of urinary bladder mucosa corresponding not only to invasive cancer but also to areas of bladder mucosa with HGIN, LGIN, and microscopically normal urothelium. Such pattern of involvement implies that the concurrent allelic losses of these markers represent early hits in bladder carcinogenesis. On the other hand LOH of marker D16S415 involves a smaller area of urinary bladder mucosa corresponding to HGIN and invasive cancer only, and so indicates that the allelic loss of this marker occurred later in urinary bladder cancer development. Histologic map code: (1) normal urothelium; (2) mild dysplasia; (3) moderate dysplasia; (4) severe dysplasia; (5) carcinoma in situ; (6) transitional cell carcinoma.

FIG. 45. Assembly of a three-dimensional display of LOH on tested chromosomes. The vertical axis represents vectors with positions of hypervariable markers and their chromosomal location. Only markers with LOH are shown. The shaded blocks represent areas of urinary bladder mucosa with LOH as they relate to progression of neoplasia represented by a histologic map of cystectomy specimen with invasive bladder cancer and adjacent precursor conditions in the background. In addition to an area of invasive cancer, there are two separate foci of non-invasive papillary TCC. The histologic map code is: NU, normal urothelium; MD, mild dysplasia; MdD, moderate dysplasia; SD, severe dysplasia; CIS, carcinoma in situ; TCC, transitional cell carcinoma. For the purpose of statistical analyses precursor conditions were grouped as follows: NM, and MdD, low-grade intraurothelial neoplasia (LGIN); MdD and CIS, high-grade intraurothelial neoplasia (HGIN).

FIG. 46.A. Identification of minimal deleted regions involved in the development and progression of bladder neoplasia by whole-organ histologic and genetic mapping—An example of a marker tested on multiple mucosal samples from the same cystectomy specimen. Marker RB1.2 is located within the RB gene and shows LOH in samples corresponding to invasive TCC and in multiple samples exhibiting changes consistent with LGIN, HGIN, as well as in samples corresponding to adjacent areas of microscopically normal urothelium (NU). Sample #1 represents allelic pattern of the same marker from peripheral blood of the same patient and is used as a control. The presence of allelic imbalance indicative of LOH was confirmed by the densitometry and is provided as OD ratio below each sample. OD≦0.5 was used as indicative of LOH.

FIG. 46.B. Example of LOH distributions superimposed on a histologic map of cystectomy specimen. Open boxes delineated by lines indicate areas of urinary bladder mucosa with alterations in a given locus. Markers shows a plaque like LOH involving almost the entire mucosa. Marker RB1 located within the RB gene shows LOH restricted to a smaller area of bladder mucosa involving invasive TCC and adjacent areas of bladder mucosa primarily with HGIN. Overall, the three makers disclosed sequential allelic losses involving the RB gene when neoplasia progresses from early to late phases of intraurothelial neoplasia and ultimately to invasive cancer. The background-shadowed area represents a histologic map of cystectomy specimen depicting distribution of various intraurothelial precursor conditions and TCC. Histologic map code: (NU) normal urothelium; (MD) mild dysplasia; (MdD) moderate dysplasia; (SD) severe dysplasia; (CIS) carcinoma in situ; (TCC) invasive transitional cell carcinoma.

FIG. 46.C. Example of chromosome 13 allelic losses in a single cystectomy specimen (map 5) with invasive non-papillary urothelial carcinoma assembled by nearest neighbor analysis. The vertical axis represents a chromosome 5 map with positions of markers and their chromosomal locations. Only altered markers are shown. The shaded blocks represent areas of urinary bladder mucosa with LOH as they relate to progression of neoplasia presented by a histologic map of cystectomy in the background. Note that several markers show LOH in a form of a plaque involving a large area of urinary bladder mucosa. Code for a histologic map is shown in B.

FIG. 46.D. Deletional map of chromosome 13 assembled from data generated by whole-organ histologic and genetic mapping. A list of all tested markers and their position according to the Cooperative Human Linkage Center Map (version 4.0) is shown. Chromosomal band locations are provided for markers with LOH only. Asterisks on the right side of the markers indicate statistically significant relationship between LOH and the development of urothelial neoplasia tested by binomial maximum likelihood analyses and calculate as logaritm of odds (LOD) scores. Bars on the left side of the chromosomal map identify the deleted regions which are defined by the positions of deleted markers and their nearest non-altered flanking markers and the predicted size of the deleted regions in centimorgans (cM). The relationship of markers with LOH to various phases of neoplasia is provided in the LOD score table shown in B. (cM, centimorgans; WOHGM, whole-organ histologic and genetic mapping of individual cystectomy specimens consecutively numbered 1 through 5. ◯—nonaltered marker, ●—markers with LOH, and Ø—noninformative marker).

FIG. 46.E. Summary of binomial maximum likelihood analysis testing the relationship among LOH in individual chromosome 5 loci and progression of urothelial neoplasia from in situ precursor conditions to invasive TCC. Cumulative LOD scores for markers with LOH were calculated at variable θ=(0.01, 0.5, and 0.99) and tested against Tmax. The significance of allelic losses in individual loci was analyzed for normal urothelium (NU); low-grade intraurothelial neoplasia (LGIN); high-grade intraurothelial neoplasia (HGIN) and transitional cell carcinoma (TCC). To simplify the data, stringency 1 calculations are presented only. The patterns of significant LOD scores are as described in Materials and Methods. Note that significant patterns of LOD scores typically parallel the high T max values. (◯—LOD score <3; ●—LOD score≧3).

FIG. 47. Cluster display of LOH patterns in progression of bladder neoplasia from intraurothelial precursor conditions to invasive cancer. The clusters of markers with LOH from all tested chromosomes were compared with the results of binomial maximum likelihood analysis. Six separate clusters were identified and are indicated by colored bars and by identical coloring of the corresponding regions of the dendrogram. The clusters contain markers with LOH distribution patterns showing no relationship to progression of bladder neoplasia (A), sporadic significant relationship to distinct phases of bladder neoplasia but no relationship to progression to invasive TCC (B), and showing statistically significant relationship to early or late of bladder neoplasia progressing to invasive TCC (C). Note that in the vast majority of markers there is concordance among the results generated by binomial maximum likelihood analysis and clustered display. Markers that show LOH distributions patterns with discrepant results are indicated by shaded areas.

FIG. 48. Summary of physical map and sequence database analysis spanning the deleted regions of chromosome 13. The Genethon positions of the markers defining the deleted regions were related to the GB4 radiation hybrid panel-based physical map. The new positions for the Genethon markers with LOH as well as flanking markers on the GB4 map were identified by electronic PCR search of BAC contigs. In addition, multiple alternative markers based on their proximity to markers with LOH were identified and added to the map. The nearest substitute markers are often located within the same BAC clone as original Genethon markers used for LOH studies. Consequently some of the original Genethon and substitute markers have the same position on the GB4 map. The original Genethon markers with LOH are shown in red while all other substitute and flanking markers are printed in black. An average EST density is provided for regions flanked by individual markers using the GB4 radiation panel map. The list of known genes within the target regions and their positions on the GB4 map is shown. In the final steps we extracted all known, proposed, and predicted genes between markers using the sequence-based mapping tools at the Ensemble website and the “Golden Path” Genome Browser and constructed final sequence-based map of the deleted chromosomal regions putatively involved in progression of bladder neoplasia from precursor conditions to invasive cancer. The sequenced-based map with positions of gene was related to SNPs map spanning the deleted regions. To simplify the diagram, only the first position of a gene sequence on the GB4 map is shown.

FIG. 49. Identification of clonal allelic losses using SNPs mapping to the RB gene containing region defined by D13S268 and D13S176 in the progression of bladder cancer through neoplastic stages.

### DESCRIPTION OF ILLUSTRATIVE EMBODIMENTS

The present invention discloses a means of detecting a neoplastic or preneoplastic phenotype for a cell or tissue based upon identification of genomic alterations employing superimposed histologic and genetic mapping. The identified markers were not only examined in paired tumor vs. normal host DNA samples, but were also related to the progression of neoplasia from precancerous lesions to invasive cancers. This was accomplished by sampling the entire mucosa of a subject bladder. The distribution of a microscopically identified invasive cancer and its precursor conditions were then displayed in the form of a histologic map. Subsequent isolation of DNA generated a set of samples in which the search for genetic alterations with various probes could be performed and the results can be superimposed over the histologic map.

Although several transforming and tumor suppressor genes have been postulated to play a role in the progression of urinary bladder cancer, specific knowledge on genome wide alterations that are involved in this process is still lacking. Herein are disclosed the evolution of genome-wide allelic losses in the progression of human urothelial neoplasia from clinically occult precursor intraurothelial conditions to invasive cancer. FIGS. 28-42 list genes that are tumor suppressor candidates. The sequence of these genes is available at GenBank at http://www.ncbi.nlm.nih.gov/entrez/query.fcgi?db=Nucleotide. Multiple DNA samples extracted from invasive bladder cancer and adjacent microscopically identified preneoplastic intraurothelial conditions of the entire organ were evaluated.

Using superimposed histologic and genetic mapping, the identified genetic alterations were matched to progressive histologic changes that paralleled the natural history of the disease. The significance of alterations in individual loci for the development and progression of urinary bladder cancer was tested by modified LOD 3 score analysis, and the data from individual chromosomes were used to assemble a genetic model of multistep urinary bladder carcinogenesis. The model represents a detailed, high density map of allelic losses on tested chromosomes in the progression of human urinary bladder cancer, providing information on the location of multiple putative tumor suppressor gene loci involved in human urinary bladder carcinogenesis.

The samples corresponded to microscopically identified intraurothelial precursor conditions ranging from dysplasia to carcinoma in situ and invasive cancer. The analysis of paired normal and tumor DNA samples disclosed allelic losses in tested hypervariable DNA markers. Subsequent use of these markers on all mucosal samples revealed that 47 had alterations with a statistically significant relation to urothelial neoplasia. The allelic losses clustered in distinct chromosomal regions, indicating the location of putative tumor suppressor genes involved in the development and progression of urinary bladder cancer. Some of the markers with statistically significant allelic losses mapped to the regions containing well characterized tumor suppressor genes, but many were located in previously unknown loci.

The majority of statistically significant allelic losses (70%) occurred early in low-grade intraurothelial dysplasia. Some of them involved adjacent areas of morphologically normal mucosa, preceding the development of microscopically recognizable precursor lesions. The remaining 30% of markers developed allelic losses in the later phases of urothelial neoplasia, implicating their involvement in progression to invasive disease. Markers exhibiting allelic losses in early phases of urothelial neoplasia could be used for detection of occult preclinical or even premicroscopic phases of urinary bladder cancer whereas markers that showed allelic losses in the later phases of the process could serve as indicators of progression to invasive disease. The disclosed genome-wide model provides important chromosomal landmarks for more specific identification of genetic changes involved in urinary bladder carcinogenesis.

For the purpose of the instant invention, the term neoplasm or neoplastic means a cell or tissue exhibiting abnormal growth, including hyperproliferation or uncontrolled cell growth, that may be benign or cancerous. The development from a normal cell to a cell exhibiting a neoplastic phenotype is a multi-step process. Cells developing a neoplastic phenotype or designated as of a cancerous cell type generally exhibit an alteration of the normal cell cycle and altered apoptotic response. Generally the changes that a cell undergoes in developing to a tumor cell may be monitored at the cellular or DNA level. Therefore, preneoplasm or preneoplastic phenotype is construed for the purposes of the instant invention to refer to a cell or tissue which exhibits changes at the DNA or cellular level that evidence the ultimate progression of the cell or tissue to a neoplastic or cancerous phenotype.

Preneoplasia is frequently characterized, for example, by dysplastic changes, particularly in the cell nucleus, that may be associated with metaplasia and carcinoma in situ. Preneoplastic conditions do not show evidence of microinvasion or other hallmarks of cancer behavior. As with the development to neoplasia, preneoplastic cells may exhibit progression through multiple steps. Although a preneoplastic cell may progress to a neoplastic stage, they may remain stable for an extended period of time and may even regress. The development of preneoplasia is often associated with enviromental factors. Examples of preneoplastic conditions in noninvasive bladder cancer include diffuse cellular atypia of the urothelium. These cells may give rise to recurrent papillomas and finally to invasive bladder cancer.

1. Nucleic Acids, Proteins and Expression of the Tumor Suppressors of the Present Invention

As used herein, the term “nucleic acid” refers to a polymer of DNA, RNA or a derivative or mimic thereof, of two or more bases in length. The term “oligonucleotide” refers to a polymer of DNA, RNA or a derivative or mimic thereof, of between about 3 and about 100 bases in length. The term “polynucleotide” refers to a polymer of DNA, RNA or a derivative or mimic thereof, of greater than about 100 bases in length. Thus, it will be understood that the term “nucleic acid” encompass the terms “oligonucleotide” and “polynucleotide”. These definitions generally refer to at least one single-stranded molecule, but in specific embodiments will also encompass at least one double-stranded molecule. Within the scope of the invention, it is contemplated that the terms “oligonucleotide”, “polynucleotide” and “nucleic acid” will generally refer to at least one polymer comprising one or more of the naturally occurring monomers found in DNA (A, G, T, C) or RNA (A, G, U, C).

Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of annealing to the nucleic acid segment being described under relatively stringent conditions such as those described herein.

Hybridization is understood to mean the forming of a double stranded molecule and/or a molecule with partial double stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and/or ionic strength of a desired stringency are determined in part by the length of the particular probe, the length and/or base content of the target sequences, and/or to the presence of formamide, tetramethylammonium chloride and/or other solvents in the hybridization mixture. It is also understood that these ranges are mentioned by way of example only, and/or that the desired stringency for a particular hybridization reaction is often determined empirically by comparison to positive and/or negative controls.

Accordingly, the nucleotide sequences of the disclosure may be used for their ability to selectively form duplex molecules with complementary stretches of genes and/or RNA. Depending on the application envisioned, it is preferred to employ varying conditions of hybridization to achieve varying degrees of selectivity of probe towards target sequence.

Nucleic acid molecules having sequence regions consisting of contiguous nucleotide stretches of about 13, 14, 15, 16, 17, 18, 20, 25, 30, 35, 40, 45, 50, 55, 60, 70, 80, 90, 100, 125, 150, 175, 200, 250, 300, 350, 400 or more basepairs (bp) to about 5000 bp, or even up to and including sequences of about 30-50 cM or so, identical or complementary to the target DNA sequence, are particularly contemplated as hybridization probes for use in embodiments of the instant invention. It is contemplated that long contigous sequence regions may be utilized including those sequences comprising about 100, 200, 300, 400, 500, 600, 700, 800, 900, 1000, 1100, 1200, 1300, 1400, 1500, 2000, 2500, 3000, 3500, 4000, 4500, 5000, 5500, 6000, 6500, 7000, 7500, 8000, 8500, 9000, 9500, 10,000 or more contiguous nucleotides or up to and including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cM.

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

For applications requiring high selectivity, it is preferred to employ relatively stringent conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe and/or the template and/or target strand, and/or would be particularly suitable for isolating specific genes and/or detecting specific mRNA transcripts. It is generally appreciated that conditions may be rendered more stringent by the addition of increasing amounts of formamide.

Preferred embodiments of the instant invention involve the detection of genetic changes in an individual by the ability of host chromosomal DNA to hybridize to a specific probe. In the context of the instant invention, probes constitute single stranded DNA of from 18 b.p. to 50 cM. It is envisioned that probes may constitute, for example, synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change which may lead to the development of a neoplastic or preneoplastic phenotype in a cell or tissue. An example of a change detectable by the failure of a probe to hybridize to a hosts chromosomal DNA is termed a loss of heterozygosity (LOH).

Tumor Suppressor Proteins

In addition to the entire sequence of a tumor suppressor whose whole or partial chromosomal deletion is indicative of cancer, the present invention also relates to fragments of the polypeptides that may or may not retain the tumor suppressing activity. Fragments, including the N-terminus of the molecule may be generated by genetic engineering of translation stop sites within the coding region. Alternatively, treatment of the tumor molecules with proteolytic enzymes, known as proteases, can produce a variety of N-terminal, C-terminal and internal fragments. Examples of fragments may include contiguous residues of the sequence of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Purification of Tumor Suppressor Proteins

It may be desirable to purify tumor suppressors whose whole or partial chromosomal deletion is indicative of cancer or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; sodium dodecyl sulfate/polyacrylamide gel electrophoresis (SDS/PAGE); isoelectric focusing. A particularly efficient method of purifying peptides is fast protein liquid chromatography (FPLC) or even HPLC.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

It is known that the migration of a polypeptide can vary, sometimes significantly, with different conditions of SDSIPAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample can be low because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. It should be possible to elute the substance without destroying the sample or the ligand. One of the most common forms of affinity chromatography is immunoaffinity chromatography. The generation of antibodies that would be suitable for use in accord with the present invention is discussed below.

The present invention also describes peptides of the tumor suppressors for use in various embodiments of the present invention. Because of their relatively small size, the peptides of the invention also can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

The present invention also provides for the use of the tumor suppressors as antigens for the immunization of animals relating to the production of antibodies. A biospecific or multivalent composition or vaccine is produced. It is envisioned that the methods used in the preparation of these compositions will be familiar to those of skill in the art and should be suitable for administration to animals, i.e., pharmaceutically acceptable.

Variants of Tumor Suppressors Whose Whole or Partial Chromosomal Deletion is Indicative of Cancer

Amino acid sequence variants of these polypeptides can be substitutional, insertional or deletion variants. Deletion variants lack one or more residues of the native protein that are not essential for function or immunogenic activity. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions are called fusion proteins.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine, and isoleucine.

Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In other embodiments, the probes or primers are labeled with radioactive species (³²P, ¹⁴C, ³⁵S, ³H, or other label), with a fluorophore (rhodamine, fluorescein) or a chemillumiscent (luciferase).

One method of using probes and primers of the present invention is in the search for genes related to tumor suppressors whose chromosomal deletion is indicative of cancer or, more particularly, orthologs of tumor suppressors whose chromosomal deletion is indicative of cancer from other species. Normally, the target DNA will be a genomic or cDNA library, although screening may involve analysis of RNA molecules. By varying the stringency of hybridization, and the region of the probe, different degrees of homology may be discovered.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In other embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, it is envisioned that the probes or primers described herein will be useful as reagents in solution hybridization, as in PCR™, for detection of expression of corresponding genes, as well as in embodiments employing a solid phase. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety. Other methods of amplication are ligase chain reaction (LCR), Qbeta Replicase, isothermal amplification, strand displacement amplification (SDA), PCR™-like template- and enzyme-dependent synthesis using primers with a capture or detector moiety, transcription-based amplification systems (TAS), cylical synthesis of single-stranded and double-stranded DNA, “RACE”, one-sided PCR™, and di-oligonucleotide amplification.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression cassette” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

Promoters and Enhancers

A “promoter” is a control sequence that is a region of a nucleic acid sequence at which initiation and rate of transcription are controlled. A promoter can be used to regulate expression of a gene, for example, in gene therapy. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Such promoters may be used to drive β-galactosidase expression for use as a reporter gene. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al., (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 2 lists several elements/promoters that may be employed, in the context of the present invention, to regulate the expression of a gene. This list is not intended to be exhaustive of all the possible elements involved in the promotion of expression but, merely, to be exemplary thereof Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al, 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et at., 1989 |
| MHC Class II HLA-DRa | Sherman et at., 1989 |
| β-Actin | Kawamoto et al., 1988; Ng et al; 1989 |
| Muscle Creatine | Jaynes et at., 1988; Horlick et at., 1989; Johnson et al., |
| Kinase (MCK) | 1989 |
| Prealbumin (Transthyretin) | Costa et al., 1988 |
| Elastasel | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et at., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., Moreau et al., 1981; Sleight et al., |
| | 1985; Firak et al., 1986; Herr et al., 1986; Imbra et al., |
| | 1986; Kadesch et al., 1986; Wang et al., 1986; Ondek et |
| | al., 1987; Kuhlet et al., 1987; Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; |
| | Hen et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Chol |
| | et al., 1988; Reisman et al., 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; |
| | | Stuart et al., 1985; Imagawa et |
| | | al., 1987, Karin et al., 1987; |
| | | Angel et al., 1987b; MeNeall et |
| | | al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai et |
| | | al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobuhn | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al, 1989, 1990a, |
| | Antigen | 1990b |
| Proliferin | Phorbol Ester-TPA | Mordacq et al., 1989 |
| Tumor Necrosis Factor | PMA | Hensel et al, 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al,. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-I (Almendro et al., 1996).

Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Specific embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

Orgins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Examples of selectable and screenable markers are well known to one of skill in the art.

Host Cells

In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAxBAc® 2.0 from INVITROGEN® and BACPACK™ BACULOVIRUS ExPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

2. Separation and Quantitation Methods

Following amplification, it may be desirable to separate the amplification products of several different lengths from each other and from the template and the excess primer for the purpose analysis or more specifically for determining whether specific amplification has occurred.

Gel Electrophoresis

In one embodiment, amplification products are separated by agarose, agarose-acrylamide or polyacrylamide gel electrophoresis using methods commonly known to one of ordinary skill in the art. (Sambrook et al., 1989).

Chromatographic Techniques

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982). In yet another alternative, labeled cDNA products, such as biotin or antigen can be captured with beads bearing avidin or antibody, respectively.

Microfluidic Techniques

Microfluidic techniques include separation on a platform such as microcapillaries, designed by ACLARA BioSciences Inc., or the LabChipTM “liquid integrated circuits” made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR™ apparatus for collection and amplification of nucleic acids from a specimen. U.S. Pat. Nos. 5,304,487 and 5,296,375, discuss devices for collection and analysis of cell containing samples and are incorporated herein by reference. U.S. Pat. No. 5,856,174 describes an apparatus which combines the various processing and analytical operations involved in nucleic acid analysis and is incorporated herein by reference.

Capillary Electrophoresis

In some embodiments, it may be desirable to provide an additional, or alternative means for analyzing the amplified genes. In these embodiment, micro capillary arrays are contemplated to be used for the analysis.

Microcapillary array electrophoresis generally involves the use of a thin capillary or channel which may or may not be filled with a particular separation medium. Electrophoresis of a sample through the capillary provides a size based separation profile for the sample. The use of microcapillary electrophoresis in size separation of nucleic acids has been reported in, for example, Woolley and Mathies, 1994. Microcapillary array electrophoresis generally provides a rapid method for size-based sequencing, PCR™ product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, for example, Jacobsen et al., 1994; Effenhauser et al., 1994; Harrison et al., 1993; Effenhauser et al., 1993; Manz et al., 1992; and U.S. Pat. No. 5,904,824, here incorporated by reference. Typically, these methods comprise photolithographic etching of micron scale channels on a silica, silicon or other crystalline substrate or chip, and can be readily adapted for use in the present invention. In some embodiments, the capillary arrays may be fabricated from the same polymeric materials described for the fabrication of the body of the device, using the injection molding techniques described herein.

Tsuda et al., 1990, describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

3. Screening

The genetic alterations or changes indicating the development of a preneoplastic phenotype or genetic changes involved in the progression or development of a neoplasm are detectable by a variety of methods, that may be utilized to identify those cells exhibiting LOH at one or more selected loci identified herein. An example of cancers that can be detected using the present invention include cancers of the brain, liver, spleen, lymph node, small intestine, blood cell, pancreatic, colon, stomach, cervix, breast, endometrium, prostate, testicle, ovary, skin, head and neck, esophagus, bone marrow cancer, lung cancer, larynx, oral tissue, kidney and esophagus, bladder, urothelial tissue, or cervix.

The following description sets forth techniques which are exemplary of means a person of ordinary skill would employ in the detection of the disclosed genetic alterations.

Gene Chips and DNA Arrays

DNA arrays and gene chip technology provides a means of rapidly screening a large number of DNA samples for their ability to hybridize to a variety of single stranded DNA probes immobilized on a solid substrate. Specifically contemplated are chip-based DNA technologies such as those described by Hacia et al., 1996 and Shoemaker et al., 1996. These techniques involve quantitative methods for analyzing large numbers of genes rapidly and accurately The technology capitalizes on the complementary binding properties of single stranded DNA to screen DNA samples by hybridization. Pease et al., 1994; Fodor et al., 1991. Basically, a DNA array or gene chip consists of a solid substrate upon which an array of single stranded DNA molecules have been attached. For screening, the chip or array is contacted with a single stranded DNA sample which is allowed to hybridize under stringent conditions. The chip or array is then scanned to determine which probes have hybridized. In a preferred embodiment of the instant invention, a gene chip or DNA array would comprise probes specific for chromosomal changes evidencing the development of a neoplastic or preneoplastic phenotype. In the context of this embodiment, such probes could include synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832 which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal; for example, luminescence. The target nucleic acid may be immobilized onto the integrated microchip that also supports a phototransducer and related detection circuitry. Alternatively, a gene probe may be immobilized onto a membrane or filter which is then attached to the microchip or to the detector surface itself In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860 both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, (1993)), the direct covalent attachment of short, 5′-phosphorylated primers to chemically modified polystyrene plates (Rasmussen, et al., (1991)), or the precoating of the polystyrene or glass solid phases with poly-L-Lys or poly L-Lys, Phe, followed by the covalent attachment of either amino- or sulfhydryl-modified oligonucleotides using bi-functional crosslinking reagents. (Running, et al., (1990); Newton, et al. (1993)). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellous membranes, the DNA probes are spotted onto the membranes. A UV light source (Stratalinker, from Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

Fluorescent In Situ Hybridization

As described in U.S. Pat. Nos. 5,427,910 and 5,523,207 which are expressly incorporated by reference, flourescent in situ hybridization (FISH) involves the introduction of a nucleic acid probe with a defined nucleotide sequence into a cell, where it preferentially hybridizes with a specific complementary nucleotide sequence of DNA, or target DNA, on one or more chromosomes within the cell. The target nucleotide sequence may be unique or repetitive, as long as it can be used to distinguish one or more specific chromosomes. The probe is labeled with a fluorescent tag so that cells with the target DNA sequence(s), to which the marked probes hybridize, can be detected microscopically. Each chromosome containing the targeted DNA sequence, and hence the hybridized probe, will emit a fluorescent signal or spot. fluorescent in situ hybridization. Thus, for example, specimens hybridized with a DNA sequence known to be contained on chromosome number 21 will produce two fluorescent spots in cells from normal patients and three spots from Down's Syndrome patients because they have an extra chromosome number 21.

Polymerase Chain Reaction

The technique of “polymerase chain reaction,” or “PCR,” as used herein generally refers to a procedure wherein minute amounts of a specific piece of nucleic acid, RNA and/or DNA, are amplified as described in U.S. Pat. Nos. 4,683,195; 4,683,202; and 4,683,194, which are herein expressly incorporated by reference. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., (1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid that is complementary to a particular nucleic acid.

Northern and Southern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

Restriction Fragment Length Polymorphism

“Restriction Enzyme Digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Incubation of about 1 hour at 37° C. is ordinarily used, but may vary in accordance with the supplier's instructions.

Restriction fragment length polymorphisms (RFLPs) analysis capitalizes on the selectivity of restriction enzymes to detect the genetic changes in specific loci. RFLP are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., Eco RI. Variations in RFLPs result from nucleotide base pair differences which alter the cleavage sites of the restriction endonucleases, yielding different sized fragments. Means for performing RFLP analyses are well known in the art.

As described in U.S. Pat. No. 5,580,729, herein expressly incorporated by reference, one means of testing for loss of an allele is by digesting the first and second DNA samples of the neoplastic and non-neoplastic tissues, respectively, with a restriction endonuclease. Restriction endonucleases are well known in the art. Because they cleave DNA at specific sequences, they can be used to form a discrete set of DNA fragments from each DNA sample. The restriction fragments of each DNA sample can be separated by any means known in the art. For example, an electrophoretic gel matrix can be employed, such as agarose or polyacrylamide, to electrophoretically separate fragments according to physical properties such as size. The restriction fragments can be hybridized to nucleic acid probes which detect restriction fragment length polymorphisms, as described above. Upon hybridization hybrid duplexes are formed which comprise at least a single strand of probe and a single strand of the corresponding restriction fragment. Various hybridization techniques are known in the art, including both liquid and solid phase techniques. One particularly useful method employs transferring the separated fragments from an electrophoretic gel matrix to a solid support such as nylon or filter paper so that the fragments retain the relative orientation which they had on the electrophoretic gel matrix. The hybrid duplexes can be detected by any means known in the art, for example, the hybrid duplexes can be detected by autoradiography if the nucleic acid probes have been radioactively labeled. Other labeling and detection means are known in the art and may be used in the practice of the present invention.

Nucleic acid probes which detect restriction fragment length polymorphisms for most non-acrocentric chromosome arms are available from the American Type Culture Collection, Rockville, Md. These are described in the NIH Repository of Human DNA Probes and Libraries, published in August, 1988. Methods of obtaining other probes which detect restriction fragment length polymorphisms are known in the art. The statistical information provided by using the complete set of probes which hybridizes to each of the non-acrocentric arms of the human genome is useful prognostically. Other subsets of this complete set can be used which also will provide useful prognostic information. Other subsets can be tested to see if their use leads to measures of the extent of genetic change which correlates with prognosis, as does the use of the complete set of alleles.

4. Methods for Treating Cancers Using Tumor Suppressors

The present invention also involves, in another embodiment, the treatment of cancer. In many contexts, it is not necessary that the cancer cell be killed or induced to undergo normal cell death or “apoptosis.” Rather, to accomplish a meaningful treatment, all that is required is that the tumor growth be slowed to some degree. It may be that the tumor growth is partially or completely blocked, however, or that some tumor regression is achieved. Clinical terminology such as “remission” and “reduction of tumor” burden also are contemplated given their normal usage. An example of cancers that can be treated with the present invention include cancers of the brain, liver, spleen, lymph node, small intestine, blood cell, pancreatic, colon, stomach, cervix, breast, endometrium, prostate, testicle, ovary, skin, head and neck, esophagus, bone marrow cancer, lung cancer, larynx, oral tissue, kidney and esophagus, bladder, urothelial tissue, or cervix.

Genetic Based Therapies

One of the therapeutic embodiments contemplated by the present inventors is the intervention, at the molecular level, in the events involved in the tumorigenesis of some cancers. Specifically, the present inventors intend to provide, to a cancer cell, an expression cassette capable of providing tumor suppressors of the present invention to that cell. Particularly preferred expression vectors are viral vectors such as adenovirus, adeno-associated virus, herpesvirus, vaccinia virus and retrovirus. Also preferred is liposomally-encapsulated expression vector.

Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

One of the methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

Adenovirus Expression Vectors

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Although it is not necessary that the adenovirus vector be replication defective, or at least conditionally defective, that type of vector is preferred. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus vectors have been used in eukaryotic gene expression (Levrero et al, 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retrovirus Expression Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Other Viral Vectors

Other viral vectors may be employed as expression constructs in the present invention. Vectors derived from viruses such as vaccinia virus (Ridgeway, 1988; Baichwal and Sugden, 1986; Coupar et al, 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al, 1988; Horwich et al, 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Non-Viral Methods for Transfer of Expression Constructs

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest also may be transferred in a similar manner in vivo and express the gene product.

In still another embodiment, the transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, WI 38, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Protein Therapy

Another therapy approach is the provision, to a subject, of tumor suppressors of the present invention, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

Combined Therapy with Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that tumor suppressor replacement therapy could be used similarly in conjunction with chemo- or radiotherapeutic intervention. It also may prove effective to combine tumor suppressor gene therapy with immunotherapy, as described above.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with a tumor suppressor expression construct and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either tumor suppressor or the other agent will be desired. Various combinations may be employed, where tumor suppressors whose chromosomal deletion is indicative of cancer is “A” and the other agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |
| A/A/B/B | A/B/A/B | A/B/B/A | B/B/A/A | B/A/B/A | B/A/A/B | B/B/B/A | |
| A/A/A/B | B/A/A/A | A/B/A/A | A/A/B/A | A/B/B/B | B/A/B/B | B/B/A/B | |

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, y-irradiation, X-rays, accelerated protons, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a tumor suppressors whose chromosomal deletion is indicative of cancer expression construct is particularly preferred as this compound.

In treating cancer according to the invention, one would contact the tumor cells with an agent in addition to the expression construct. This may be achieved by irradiating the localized tumor site with radiation such as X-rays, accelerated protons, v-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with a tumor expression construct, as described above.

Agents that directly cross-link nucleic acids, specifically DNA, are envisaged to facilitate DNA damage leading to a synergistic, antineoplastic combination with tumor suppressors whose chromosomal deletion is indicative of cancer. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, accelerated protons, and/or the directed delivery of radioisotopes to tumor cells. Other forms of DNA damaging factors are also contemplated such as microwaves, and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics Standards.

The inventors propose that the regional delivery of tumor expression constructs to patients with cancers will be a very efficient method for delivering a therapeutically effective gene to counteract the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining tumor suppressors therapies with chemo- and radiotherapies, it also is contemplated that combination with other gene therapies will be advantageous. For example, targeting of multiple tumor suppressors deletions at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fins, jun, trk, ret, gsp, hst, bcl and abl.

It also should be pointed out that any of the foregoing therapies may prove useful by themselves in treating cancer. In this regard, reference to chemotherapeutics and non-tumor suppressor gene therapy in combination should also be read as a contemplation that these approaches may be employed separately.

Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. The routes of administration will vary, naturally, with the location and nature of the lesion, and include, e.g., intradermal, transdermal, parenteral, intracranial, intravenous, intramuscular, intranasal, subcutaneous, percutaneous, intratracheal, intraperitoneal, intratumoral, perfusion, lavage, direct injection, and oral administration and formulation. In the present invention, intracranial or intravenous administration are preferred embodiments. Administration may be by injection or infusion. Please see Kruse et al. (J. Neuro-Oncol., 19:161-168, 1994), specifically incorporated by reference, for methods of performing intracranial administration. Such compositions would normally be administered as pharmaceutically acceptable compositions, described supra.

Intratumoral injection, or injection into the tumor vasculature is specifically contemplated for discrete, solid, accessible tumors. Local, regional or systemic administration also may be appropriate. For tumors 1.5 to 5 cm in diameter, the injection volume will be 1 to 3 cc, preferably 3 cc. For tumors greater than 5 cm in diameter, the injection volume will be 4 to 10 cc, preferably 5 cc. Multiple injections delivered as single dose comprise about 0.1 to about 0.5 ml volumes, preferable 0.2 ml. The viral particles may advantageously be contacted by administering multiple injections to the tumor, spaced at approximately 1 cm intervals. In an average administration, 10³ to about 10¹⁵ viral particles may be given to the patient.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising the adenovirus. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

The adenovirus also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulaitons include such typical excipients as, for example, pharmaceutical grades of mannitol, lactose, starch, magnesium stearate, sodium saccharine, cellulose, magensium carbonate and the like. The compositions take the form of solutions, suspensions, tablets, pills, capsules, sustained release formulations or powders. When the route is topical, the form may be a cream, ointment, salve or spray.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A Genome Wide Map of LOH Markers For Bladder Cancer

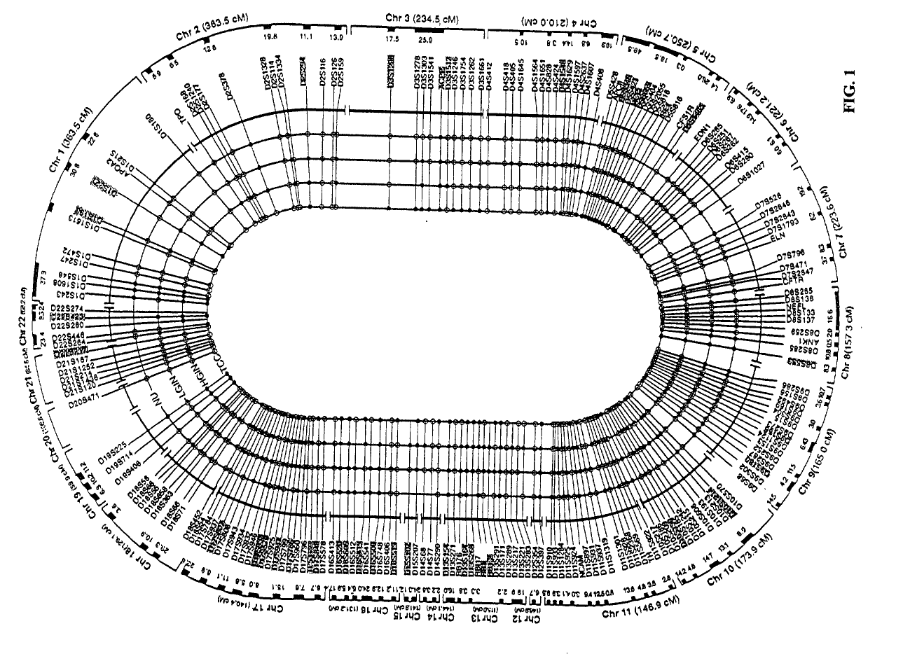

Assembled data obtained from individual chromosomes was utilized to produce a model of multistep bladder carcinogenesis (FIG. 1). Assembling the whole-organ histologic and genetic mapping data from all chromosomes allows the analysis of the genome-wide patterns of allelic losses in relation to progression of bladder neoplasia from precursor intraurothelial conditions to invasive cancer. Using this approach we identified those chromosomal regions which were involved in early occult phases of bladder neoplasia and those that are relevant for the development of more advanced disease such as severe dysplasia or carcinoma in situ progressing to clinically aggressive invasive cancer.

The relationship among the clonal allelic losses and development of urothelial neoplasia in whole bladder mucosa was initially tested by the binomial maximum likelihood analysis. Of 194 markers with LOH, 122 showed clonal allelic losses that could be related to the development or progression of intraurothelial neoplasia. Overall, 83 (41.5%) of these markers exhibited LOH associated with the expansion of early in situ preneoplastic conditions such as mild to moderate dysplasia, while 26 (13.0%) of markers with LOH could be related to the development of severe dysplasia/carcinoma in situ progressing to invasive cancer. The allelic losses related to clonal expansion of intraurothelial neoplasia and its progression to invasive cancer clustered in 59 distinct chromosomal regions suggesting that these regions may contain tumor suppressor genes involved in bladder carcinogenesis.

In order to further verify the significance of this data the distribution patterns of allelic losses were clustered using hierarchical command and compared with the results of binomial likelihood analysis. This permitted separation of all markers into two major groups i.e. those with no relationship of their allelic losses to progression of neoplasia and those that showed statistically significant association with various phases of neoplasia. A large heterogeneous cluster of markers with the relationship among their clonal allelic losses and the development or progression of bladder neoplasia could be farther sub-classified into two groups. The first group consisted mainly of markers with only limited relationship to distinct phases of bladder neoplasia. The other was comprised predominately of markers with clonal allelic losses involved in both early and late phases of bladder neoplasia implicating their role in bladder cancer progression. Overall, there was a concordance among the results generated by binomial likelihood and hierarchical clustering analyses, however, each cluster contained a significant proportion of markers with discrepant results.

In general, this data is in keeping with recent results generated by comparative genomic hybridization techniques and disclosed enormous complexity and redundancy of genetic hits that could be identified even in early phases of bladder carcinogenesis. This together with the discrepant results for many markers with clonal allelic losses disclosed by comparison of hierarchical clustering command and binomial likelihood analysis suggests that such approach was not sufficiently stringent and most likely overestimated the putative functional involvement of many chromosomal regions for disease development and progression.

When the genome-wide distribution of allelic losses was analyzed by the nearest neighbor algorithm it turned out that all areas of bladder mucosa involved by LOH were geographically related. Even those markers that exhibited allelic losses involving several separate areas of bladder mucosa and could not be related to any specific phases of bladder neoplasia were in fact located within larger plaques of clonal allelic loses mapping to other chromosomal regions. This indicated that such losses represented secondary apparently random events occurring within the preexisting clone of genetically abnormal urothelial cells that occupied a large area of bladder mucosa. On the other hand, the initial visual analysis of this data disclosed that early clonal expansion of abnormal urothelial cells involving large areas of bladder mucosa was associated with several concurrent clonal hits involving distinct regions of different chromosomes. Interestingly, in several instances the neighboring markers mapping to the same chromosomal region showed synchronous allelic losses involving almost the entire bladder mucosa. This observation suggested that the stringency of our analysis and consequently of the pathogenetic relevance of our data could be increased by the algorithms searching for overlapping plaques of clonal allelic losses with strict geographical relationship to early and late phases of bladder neoplasia. Such changes may signify incipient genetic hits with synergistic affect causing intraurothelial expansion of preneoplastic clone and those relevant for its subsequent progression to clinically aggressive invasive disease.

The model shows the evolution of LOH in individual loci and their significance for the development and progression of urothelial neoplasia as revealed by the LOD scores. Many of the markers with LOH showed statistically significant alterations in relation to development or progression of intraurothelial neoplasia. The markers with significant LOD score linking the allelic losses to different phases of urothelial neoplasia clustered in distinct chromosomal regions, identifying these regions as positions of putative tumor suppressor genes. The major advantage of this superimposed histologic and genetic mapping technique is that it included the entire mucosa of the affected bladder in the analysis. Markers exhibiting LOH associated with early clonal expansion involved large areas of urinary bladder mucosa and could be used as powerful tools to monitor the preclinical and premicroscopic phases of urothelial neoplasia in histologic samples and voided urine sediments. Those markers that exhibited statistically significant LOH in more advanced phases of urothelial neoplasia, such as high-grade intraurothelial neoplasia progressing to invasive disease, could be used as markers of a high risk of progression to invasive cancer in clinically occult phases of in situ neoplasia.

The model disclosed can be used in conjunction with the human genome data, significantly facilitating the identification of new target genes and generating a large number of novel markers for early cancer detection. Thus, the minimally deleted regions involved in the development and progression of bladder neoplasia identified by whole-organ histologic and genetic mapping were defined based on markers from the sex-averaged genetic recombination map from Marshfield. These markers were then reoriented with the physical map markers used to generate the radiation hybrid-based GeneMap99. Produced by the International Radiation Hybrid Mapping Consortium, GeneMap99 represents the most complete melding of microsatellite and EST markers mapped against the GB4 and G3 radiation hybrid panels. Further conversion to a purely physical, BAC-based map was accomplished by correlating BAC clone marker content, using electronic PCR, with the emerging whole genome assembly reflected in the “Golden Path” and Ensembl genome browsers. Because a number of the original Marshfield markers could not be found in GeneMap99, substitutes were required and were proposed based on numerous factors. These included nearest neighbor markers chosen from the Marshfield map, BLAST searches using marker PCR primers against both complete and “working draft” sequence submitted to GenBank, nearest neighbor markers based on the genome browsers, or physical distance estimations when other resources failed to provide candidates. The Baylor College of Medicine Search Launcher provided the portal and integration for these links.

To construct the final gene map, we extracted all known, proposed and predicted genes between markers using the GeneWise and Genscan output as displayed in tracks on the Ensemble and Golden Path Genome Browser websites respectively. In the final steps, the positions of all currently defined SNPs mapping within 10 kb of each gene were integrated with the gene map. This provided the most accurate currently available map of all known proposed and predicted genes as well and SNPs mapping to the deleted chromosomal regions putatively involved in development and progression of human bladder neoplasia.

The most promising putative tumor suppressor gene loci were selected for further characterization and development of markers for early detection. The following paragraphs describe in more detail the target loci on individual chromosomes selected for this purpose. Though virtually every chromosome can be altered in urinary bladder cancer, only those alterations of individual chromosomes that are involved in the early phases of neoplasia or its progression to invasive disease were selected, a representative population of which are set forth in Table 4.

TABLE 4
Target Putative Tumor Suppressor Gene Loci

| | | | | Predicted |
|---|---|---|---|---|
| | | | | Length of |
| | | | Markers | Deleted |
| | Early Event (NU - LGIN) | | with | Segment |
| Location | Late Event (HGIN - TCC) | Flanking Markers | LOH | (cM) |
| Chromosome 1 | | | | |
| 1p31-32 | Early Event | D1S207-D1S1613 | D1S198 | 30.8 |
| 1p35-36 | Early Event | D1S450-D1S160 | D1S548 | 37.9 |
| 1p35-36 | Early Event | D1S450-D1S160 | D1S1608 | 37.9 |
| 1p35-36 | Early Event | D1S450-D1S160 | D1S243 | 37.9 |
| 1q20-21 | Early Event | D1S223-D1S418 | D1S221 | 16.9 |
| 1q22 | Late Event | SPTA1-D1S318 | APOA2 | 22.8 |
| Chromosome 2 | | | | |
| 2p16-21 | Early Event | D2S136-D2S123 | D2S378 | 12.8 |
| 2p23-24 | Late Event | D2S272-D2S131 | D2S1240 | 6.5 |
| 2p25 | Late Event | D2S207 | TPO | 5.9 |
| 2q14-21 | Early Event | D2S95-D2S1334 | D2S114 | 19.8 |
| 2pter-2qter | Early Event | D2S156-D2S326 | D2S294 | 11.1 |
| 2q36 | Early Event | D2S126-D2S172 | D2S159 | 13.9 |
| Chromosome 3 | | | | |
| 3p21.3 | Late Event | D3S1100-3S1277 | D3S1298 | 9.9 |
| 3p13.3 | Late Event | D3S1541-3S1512 | D3S1278 | |
| 3p13.3 | Early Event | D3S1541-3S1512 | D3S1303 | |
| 3p13.3 | Late Event | D3S1541-3S1512 | D3S1541 | |
| 3q21-25.3 | Early Event | D3S1541-3S1512 | ACPP | |
| 3q21-25.3 | Late Event | D3S1541-3S1512 | D3S1512 | |
| 3p26.2-27 | Late Event | D3S1591-3S1311 | D3S1246 | |
| 3p26.2-27 | Late Event | D3S1591-3S1311 | D3S1754 | |
| 3p27-28 | Late Event | D3S1591-3S1311 | D3S1262 | |
| 3p27-28 | Late Event | D3S1591-3S1311 | D3S1661 | |
| Chromosome 4 | | | | |
| 4p13-14 | Early Event | D4S2369-4S2629 | D4S405 | 10.5 |
| 4q26-27 | Early Event | D4S1611-D4S427 | D4S828 | 3.8 |
| 4q31 | Early Event | D4S424-D4S1629 | D4S1548 | 14.4 |
| 4q32-34 | Early Event | D4S1626-D4S243 | D4S1597 | 6.8 |
| 4q34-35 | Early Event | D4S499-D4S171 | D4S1607 | 19.9 |
| 4q34-35 | Early Event | D4S499-D4S171 | D4S408 | 19.9 |
| Chromosome 5 | | | | |
| 5q14-22 | Late Event | D5S424-D5S656 | D5S428 | 48.4 |
| 5q14-22 | Late Event | D5S424-D5S656 | APCII | 48.4 |
| 5q14-22 | Late Event | D5S424-D5S656 | D5S346 | 48.4 |
| 5q14-22 | Early Event | D5S424-D5S656 | D5S421 | 48.4 |
| 5q14-22 | Early Event | D5S424-D5S656 | MCC | 48.4 |
| 5q23.1 | Early Event | D5S424-D5S656 | D5S659 | 48.4 |
| 5q23.3 | Early Event | D5S424-D5S656 | D5S404 | 48.4 |
| 5q23.3 | Early Event | D5S656-D5S808 | D5S2055 | 18.5 |
| 5q23.3 | Early Event | D5S656-D5S808 | D5S818 | 18.5 |
| 5q31.1-31.3 | Early Event | D5S656-D5S808 | IRF1 | 18.5 |
| 5q31.1-31.3 | Early Event | D5S816-SPARC | CFS1R | 25.7 |
| 5q35.2 | Early Event | IG22-D5S1456 | D5S1465 | 1.4 |
| Chromosome 6 | | | | |
| 6p23-24 | Early Event | D6S399-D6S470 | EDN1 | 6.9 |
| 6q14 | Early Event | D6S286-D6S482 | D6S251 | 17.6 |
| 6q22-23 | Early Event | D6S407-D6S270 | D6S262 | 14.9 |
| 6q24-25 | Early Event | D6S441-D6S473 | D6S290 | 6.1 |
| 6q27 | Early Event | D6S503 | D6S1 027 | 6.0 |
| Chromosome 7 | | | | |
| 7p15.1-21 | Early Event | D7S1808-7S2846 | D7S526 | 13.3 |
| Chromosome 8 | | | | |
| 8p11-21 | Early Event | D8S283-D8S298 | D8S259 | 18.6 |
| 8p11 | Late Event | D8S283 | D8S137 | 18.6 |
| 8p11 | Early Event | D8S283 | D8S133 | 18.6 |
| 8p11 | Late Event | D8S283 | D8S136 | 18.6 |
| 8p11.2 | Early Event | D8S567-D8S268 | ANK1 | 0.5 |
| 8q11-12 | Early Event | PENK-D8S507 | D8S285 | 10.8 |
| 8pter-8qter | Early Event | D8S260-D8S84 | D8S553 | 8.3 |
| Chromosome 9 | | | | |
| 9p11-13 | Early Event | D9S165-D9S52 | D9S304 | 3.0 |
| 9p22-23 | Early Event | D9S285-D9S268 | D9S156 | 3.6 |
| 9p23-24 | Early Event | D9S268-D9S199 | D9S286 | 10.7 |
| 9q12-13 | Late Event | D9S200-D9S175 | D9S273 | 10.7 |
| 9q12-13 | Early Event | D9S200-D9S175 | D9S166 | 10.7 |
| 9q13-22 | Early Event | D9S152-D9S318 | D9S252 | 11.5 |
| 9q22.3 | Late Event | D9S151-D9S176 | D9S287 | 4.2 |
| 9q22.3 | Early Event | D9S151-D9S176 | D9S180 | 4.2 |
| 9q34.1-34.3 | Early Event | ABL1-D9S158 | D9S66 | 14.5 |
| Chromosome 10 | | | | |
| 10p11-12 | Early Event | D10S193-10S611 | D10S213 | 6.9 |
| 10p11-12 | Early Event | D10S193-10S611 | D10S1214 | 6.9 |
| 10q23 | Early Event | D10S676-10S607 | D10S606 | 13.1 |
| 10q23 | Early Event | D10S201-10S185 | D10S215 | 14.7 |
| 10q23 | Late Event | D10S201-10S185 | D10S1242 | 14.7 |
| 10q25-26 | Early Event | D10S221-10S209 | D10S190 | 4.6 |
| 10q26 | Early Event | D10S186-10S1134 | D10S217 | 14.2 |
| Chromosome 11 | | | | |
| 11p13 | Early Event | D11S1290-WT1 | D11S1301 | 13.6 |
| 11q15.1-15.2 | Late Event | D11S928-D11S902 | D11S2368 | 4.8 |
| 11q15.2-15.4 | Early Event | D11S926-D11S4465 | D11S569 | 3.6 |
| 11p15.5 | Late Event | D11S1318-HRAS1 | D11S922 | 2.6 |
| 11q13.3-13.4 | Late Event | D11S911-D11S1396 | D11S937 | 0.8 |
| 11q14.1-14.3 | Early Event | D11S901-D11S900 | D11S931 | 12.5 |
| 11q22.3-23.1 | Early Event | D11S2000-NCAM | D11S897 | 9.4 |
| 11q23.2-23.3 | Early Event | CD3D-D11S925 | D11S924 | 3.0 |
| 11q23.3-24 | Early Event | D11S1345-D11S934 | D11S1284 | 8.0 |
| 11q23.3-24 | Early Event | D11S1345-D11S934 | D11S933 | 8.0 |
| 11q24-25 | Early Event | D11S912-D11S1304 | D11S910 | 9.5 |
| Chromosome 12 | | | | |
| 12p1 3.2-13.3 | | D12S356-D12S77 | D12S397 | 6.7 |
| Chromosome 13 | | | | |
| 13q12.1 | | | D13S221 | |
| 13q12.1-12.3 | Late Event | D13S260-D13S267 | D13S171 | 3.2 |
| 13q14.1-14.3 | Early Event | D13S263-D13S284 | D13S291 | 4.8 |
| 13q14.1-14.3 | Early Event | D13S263-D13S284 | RB1 | 4.8 |
| 13q14.1-14.3 | Late Event | D13S263-D13S284 | RB1.2 | |
| 13q14.1-14.3 | Early Event | D13S263-D13S284 | D13S164 | 4.8 |
| 13q14.1-14.3 | Early Event | D13S263-D13S284 | D13S268 | 4.8 |
| 13q22-31 | Early Event | D13S170-D13S266 | D13S271 | 4.0 |
| 13q32 | Early Event | | D13S154 | |
| Chromosome 14 | | | | |
| 14q23 | Late Event | D14S592-D14S63 | D14S290 | 2.2 |
| 14q31 | Late Event | D14S616-D14S67 | D14S68 | 3.6 |
| Chromosome 15 | | | | |
| 15q26.1-26.2 | Early Event | D15S230-FES | D15S207 | 24.3 |
| 15q26.1-26.2 | Late Event | D15S87-D15S230 | D15S107 | 12.1 |
| Chromosome 16 | | | | |
| 16p13.2-13.3 | Late Event | D16S406-D16S418 | D16S513 | 1.2 |
| 16p13.1 | Early Event | D16S287-D16S748 | D16S500 | 12.9 |
| 16q11.2-12.1 | Early Event | D16S514-D16S409 | D16S541 | 24.0 |
| 16q11.2-12.1 | Early Event | D16S514-D16S409 | D16S415 | 24.0 |
| 16q22 | Early Event | D16S515-D16S496 | D16S512 | 5.4 |
| 16q24 | Early Event | D16S511-D16S507 | D16S505 | 5.9 |
| 16q24 | Early Event | D16S413-D16S402 | D16S520 | 17.4 |
| Chromosome 17 | | | | |
| 17p12 | Early Event | D17S921-D17S520 | D17S947 | 15.1 |
| 17p12 | Early Event | D17S921-D17S520 | D17S799 | 15.1 |
| 17p13.1 | Early Event | D17S945-D17S796 | D17S786 | 7.6 |
| 17p13.1 | Late Event | D17S945-D17S796 | D17S960 | 7.6 |
| 17p13.1 | Late Event | D17S945-D17S796 | TP53 | 7.6 |
| 17p13.1-13.3 | Late Event | D17S938-D17S926 | D17S849 | 8.7 |
| 17p13.1-13.3 | Late Event | D17S938-D17S926 | D17S578 | 8.7 |
| 17q11.2-12 | Early Event | D17S250-D17S946 | D17S579 | 8.0 |
| 17q11.2-12 | Late Event | D17S250-D17S946 | D17S933 | 8.0 |
| 11q21.1-21.32 | Early Event | D17S946-D17S931 | D17S932 | 5.8 |
| 11q21.1-21.32 | Late Event | D17S946-D17S931 | D17S934 | 5.8 |
| 11q21.33-22 | Early Event | D17S931-D17S809 | D17S943 | 7.1 |
| 17q24.1-24.3 | Early Event | D17S944-D17S795 | D17S807 | 6.9 |
| 17q25.1-25.3 | Late Event | D17S937-D17S928 | D17S784 | 22.6 |
| Chromosome 18 | | | | |
| 18p11.2-11.31 | Late Event | D18S53-D18S976 | D18S452 | 29.3 |
| 18p11.2 | Late Event | D18S53-D18S976 | D18S66 | 29.3 |
| 18q22 | Early Event | D18S483-D18S55 | D18S68 | 3.6 |
| Chromosome 19 | | | | |
| 19p13.2-13.3 | Late Event | D19S916-D19S1034 | D19S406 | 6.3 |
| 19p13.1 | Late Event | D19S199-D19S221 | D19S714 | 10.2 |
| 19q13.1 | Early Event | D19S425-D19S433 | D19S225 | 11.2 |
| Chromosome 21 | | | | |
| 21q22 | Late Event | | D21S212 | |
| Chromosome 22 | | | | |
| 22p11.1-11.2 | Early Event | D22S421-D22S311 | D22S264 | 23.4 |
| 22p11.1-11.2 | Late Event | D22S421-D22S311 | D22S446 | 23.4 |
| 22q12.2-12.3 | Late Event | D22S278-D22S275 | D22S280 | 8.3 |
| 22q13.1-13.2 | Early Event | D22S282-D22S279 | D22S423 | 2.4 |

In the progression of neoplastic lesions, low grade, superficially growing papillary lesions of the bladder have, in general fewer chromosomal changes than high-grade invasive carcinoma and are characterized by frequent trisomies of chromosome 1 and 7 and deletions of chromosome 9. High-grade invasive bladder carcinomas develop multiple cumulative rearrangements with deletions of chromosomes, and formation of marker chromosomes that frequently involve chromosomes 3, 4, 8, 9, 10, 11, 13, and 17.

An exemplary list of such alterations, including LOH and allelic loss, on chromosomes 3, 4, 9, 11 and 17 is representative of analogous events on other chromosomes in the development of urothelial neoplasia.

Chromosome 3: Nonrandom deletions of chromosome 3, especially loss of 3p, is a hallmark of renal cell carcinoma and a frequent denominator of several common human malignancies. Mapping studies have identified several putative tumor suppressor gene loci on the short arm of chromosome 3 involved in solid tumors, and several target genes mapped to this region have been implicated in the biology of human malignancies. Cytogenetic observations indicate that chromosome 3 is also involved in urinary bladder cancer. In an in vitro system, progressive nonrandom deletions of 3p, 11 p, and 13q appeared in human urothelial cells. Deletions on 3p, 5q, and 17p correlate with the development of a high-grade invasive bladder cancer and were more often seen in advanced tumor stages. Comparative genomic hybridization studies have identified gains and losses of genetic material on the p and q arms of chromosome 3 in urinary bladder tumors. Functional studies have shown that the deletion of 3p13-14.2 was associated with immortalization of human urothelial cells. The data imply that chromosome 3 contains important genes involved in the development of urinary bladder cancer.

Chromosome 4: Molecular mapping studies and the assembly of maps of chromosome 4 provide important clues on the location of several target gene and loci implicated to play a role in the development of human cancer. Recent comparative genomic hybridization and hypervariable DNA marker studies have shown that chromosome 4 may contain important genes for the development of urinary bladder cancer. LOH of at least one marker mapped to chromosome 4 could be identified in approximately 45% of bladder tumors. These studies also indicate at least two putative tumor suppressor gene loci on the p and q arms of chromosome 4 are involved in urinary bladder carcinogenesis and are predominantly involved in the progression of bladder neoplasia to high grade invasive cancer. These regions were subsequently better defined by the fluorescence in situ hybridization studies. Further characterization of loci on chromosome 4 may provide important information on early mechanisms of development of aggressive variants of bladder cancer. The development of FISH and hypervariable DNA probes for these loci for early detection of clinically occult urinary bladder cancer would be of particular importance. Superimposed histologic and genetic mapping studies identified three well-defined clusters of allelic losses involving the p and q arms that may represent early events in the development of urinary bladder neoplasia.

Chromosome 8: Alterations of chromosome 8, especially of the p arm, are frequently observed in urinary bladder cancer. Clonal alterations of this chromosome were linked by early cytogenetic studies to high-grade aggressive variants of urinary bladder cancer. Recent studies with hypervariable DNA markers identified allelic losses in several specific regions of both arms of chromosome 3. The gains of DNA sequences were reported on the q arm of chromosome 8 by comparative genomic hybridization studies. Particularly high levels of amplification restricted to 8q21-22 were identified in a small percentage of high-grade bladder tumors.

Chromosome 11: Allelic losses of chromosome 11 involving large portions of the p and q arms are among the most frequent alterations found in solid tumors including urinary bladder cancers. The involvement of chromosome 11 seems to have a somewhat similar pattern to the involvement of chromosome 9 i.e., large portions of both arms are frequently missing in urinary bladder tumors. Similar to early cytogenetic studies, hypervariable marker and comparative genomic hybridization studies have linked the allelic losses of chromosome 11 to high-grade, clinically aggressive bladder tumors. Some of these studies defined several distinct regions of losses or amplifications which may contain transforming or tumor suppressor genes. The analysis of allelic losses on chromosome 11 by superimposed histologic and genetic mapping studies helped to define tumor suppressor gene loci located on both arms of chromosome 11 and relate them to the development of early phases of urinary bladder neoplasia.

Chromosome 17: Alterations of chromosome 17, especially of the p arm, involving the p53 locus are among the most frequent alterations in many human malignancies, including urinary bladder tumors. More recent studies indicate that other genes mapped to chromosome 17 may play a critical role in the development of distinctive tumor types. Alterations of this chromosome were linked to urinary bladder cancer progression and development of high-grade invasive tumor, making it a potential target for early detection of clinically aggressive variants of urinary bladder neoplasia. Chromosome 17 shows a unique pattern of allelic losses in relation to progression of urothelial neolasia from intraurothelial precursor conditions to invasive cancer, the increased number of allelic losses in several specific loci parallelling the progression of intraurothelial precursor conditions from mild dysplasia to carcinoma in situ. Mapping studies and the use of superimposed histologic and genetic mapping including the p53 gene identified several additional putative tumor suppressor gene loci on this chromosome, described in detail below.

### Example 2

### Target Tumor Suppressor Gene Loci on Chromosome 3

Analysis of allelic losses on chromosome 3 in relation to the progression of urothelial neoplasia and their subsequent testing on voided urine and bladder tumor samples disclosed a putative tumor suppressor gene locus in the q21-23 region frequently involved in urinary bladder carcinogenesis. The minimal deleted region was 11 cM long and centered around the ACPP marker, flanked by D3S1541 and D3S1512 microsatellites. The results of superimposed histologic and genetic mapping indicated that the ACCP locus is involved in early precursor phases of urothelial neoplasia, its alteration perhaps preceding microscopically recognizable changes. The allelic losses were identified in more than 30% of urinary bladder tumor samples and more than 50% of voided urine samples of patients with TCC. Moreover, they appeared in voided urine of patients with a history of TCC but no microscopically or clinically detectable lesions at the time of testing. The allelic losses in the other parts of the chromosome, though frequent, did not form a clearly defined locus. The ACPP gene mapped to the 3q21-23 region codes for prostatic specific acid phosphatase, which is used as a tissue-specific marker in the diagnosis of prostatic cancer. The gene is not expressed in normal or neoplastic urothelial cells, and its involvement in pathogenesis of urothelial neoplasia is very unlikely. The high frequency of LOH in the ACPP gene locus in urinary bladder cancer suggests rather the presence of an as yet unknown tumor suppressor gene or genes in its vicinity. The results of superimposed histologic and genetic mapping studies on chromosome 3 are described in detail below.

Using YAC clone contig data mapped to this locus, limited screening of YACs was performed for allelic losses in the ACPP locus. A FISH probe was developed with a YAC 832b insert. The probe identified allelic losses in touch prints of approximately 30% of tested bladder tumors. Screening of the CEPH/BAC clone library with the most frequently deleted markers (ACPP, D3S152) mapped to the 3q21-23 locus identified a single BAC522C10 clone. This clone was used to develop a more efficient probe for this locus and to identify a target tumor suppressor gene located in this region (FIG. 26). The further identification of the q21-23 locus and its target tumor suppressor gene locus and the development of probes for early detection of urinary bladder cancer based on LOH in this locus is contemplated within the scope of the present invention. The locus in the p21 region centered around D3S1277 was infrequently altered (less than 10% of the cases) and therefore was not selected for further characterization and development of biomarkers.

### Example 3

### Isolation of Genomic BAC Clones and FISH Analysis

Human genomic BAC libraries from California Institute of Technology (Research Genetics) were screened by RCR using the primers for two most frequently deleted markers—ACPP and D3S152. Using this approach a single BAC 522C10 was identified that was positive for D3S152 marker. No BAC positive for the ACPP marker was identified. BAC522C10 was labeled with digoxigenin-11-DUTP by nick translation using the Nick translation kit (Gibco/BRL) and used for FISH analysis of 21 bladder tumors. Touch preparations of fresh bladder tumor samples were treated with HCl-Triton 100/formaldehyde and washed with 2× SSC. Cytospin preparations of normal urothelial cells obtained by scraping of urethers from nephrectomy specimens were used as controls. For FISH analyses we a BAC 522C10 probe was co-hybridized with a chromosome 3-specific alpha satellite probe labeled with spectrum orange (Vysis). The hybridization was carried out overnight at 37° C. Digoxigenin-labeled-probes were detected by FITC conjugated sheep anti-digoxigenin antibody. Samples were counterstained with DAPI/antifade and analyzed using a LEICA fluorescence microscope equipped with appropriate sets of filters for visualizing spectrum green and orange as well as DAPI counterstain. Approximately 100 nuclei with signals from each probe were scored. The slides were analyzed only if approximately 80% of the cells were interpretable in the field of view. Only non-overlapping, intact nuclei were scored. Split centromeric signals (distance between two signals is equal or less than 0.5 μm) were counted as one, and minor centromeric signals were disregarded. For photographic documentation the images were collected on a Zeiss fluorescence microscope equipped with a Ho-mamatsu high resolution/sensitivity CCD video camera and digitally processed using Adobe PhotoShop.

### Example 4

### Statistical Analysis

For the purpose of statistical analysis the intraurothelial precancerous changes were classified into two major groups: low-grade intraurothelial neoplasia (mild and moderate dysplasia) and high-grade intraurothelial neoplasia (severe dysplasia and carcinoma in situ). The relationship between alterations of the markers and urothelial neoplasia was tested by chi-square contingency tables, ROC analysis and LOD score tests. For the purpose of the analysis of this data, the ROC and LOD score tests were performed as follows.

In a typical ROC analysis a tested parameter is compared with another variable that represents a “gold standard” (Swets and Pickett, 1982). In this analysis the tested parameters represent the alterations of the markers compared with the microscopic identification of the urothelial changes. This yielded a 2×4 contingency table (fji; j=1,2; i=1, . . . ,4). The columns designated whether the marker was unchanged or changed and the 4 grades represented the microscopic status of the urothelium (D0 normal urothelium, D1 low-grade intraurothelial neoplasia, D2 high grade intraurothelial neoplasia, and D3, TCC). To calculate the ROC curve, 3 contingency subtables were formed summing the columns below a cutoff i (D0, . . . ,Di-1), and above (Di, . . . ,D3) for i=1, . . . ,3. For each table the false-negative fraction (FNF), true negative fraction (TNF), false-positive fraction (FPF) and true positive fraction (TPF) were calculated as:

FNF=(f21+ . . . +f2i)/f2·

TNF=(f11+ . . . +f1i)/f1·

FPF=(f1i+ . . . +f13)/f1·

TPF=(f2i+ . . . +f23)/f2·

Typically, an ROC curve is a plot of FPF on the x axis and TPF on the y axis augmented with the two end points (0,0) and (1,1) from which a curve is estimated on the basis of probit theory (Metz, 1989). The analysis of the present data was performed by plotting the complement TNF vs FNF. This provided a deviation of the ROC curves from the guess line in agreement with the progression of neoplasia by placing normal urothelium in the lower left and TCC in the upper right of the curve. The ROC analysis was performed with the use of the ROCFIT program by Metz (Metz, 1989). The significance of the areas below the ROC curves vs the guess line were examined by t-test. The differences among the areas of the ROC curves were tested by the Tukey multiple comparison test at p=0.05.

For the LOD score analysis, the data were organized using the same 2×4 table. For each category of urothelial status (Di; i=0, . . . ,3) the maximum likelihood for the binomial distribution was used to determine whether a row of data was consistent with a hypothesis of an unchanged (all negative) marker by calculating the log likelihood with

$$li = ln ( \frac{{{θ}_{i}^{{f}_{1 i}} ( 1 - {θ}_{i} )}^{{f}_{2 i}}}{{{\hat{θ}}_{i}^{{f}_{1 i}} ( 1 - {\hat{θ}}_{i} )}^{{f}_{2 i}}} )$$

for the null hypothesis H0, where θ=θₗ and \hat{θ}=f₁ₗ/(f₁ₗ+f₂ₗ) is the maximum likelihood estimate of a negative marker. Two times the negative of the log likelihood, −2li, is asymptotically chi-square with 1 degree of freedom, χ2(1). This expression can be written

$$- 2 {l}_{i} = 2 ln ( 10 ) {log}_{10} ( \frac{{{\hat{θ}}_{i}^{{f}_{1 i}} ( 1 - {\hat{θ}}_{i} )}^{{f}_{2 i}}}{{{θ}_{i}^{{f}_{1 i}} ( 1 - {θ}_{i} )}^{{f}_{2 i}}} ) \\ = 2 ln ( 10 ) LOD ( {\hat{θ}}_{i} : {θ}_{i} )$$

where LOD(\hat{θ}ₗ:θᵢ) is the LOD-score function evaluated at θᵢ (Ott, 1991). Each row of the table for which −21i, have approximate χ2(1) can be tested separately (stringency level 1) or all rows for diagnosis Di and more advanced (Di, . . . ,D3) can be combined (stringency level 2) to get

$$\begin{matrix}\begin{matrix}{f}_{1 i +} = {\sum }_{j = i}^{3} {f}_{1 j} \\ {f}_{2 i +} = {\sum }_{j = i}^{3} {f}_{2 j}\end{matrix} \\ LOD ( {\hat{θ}}_{i +} : {θ}_{i +} ) = {log}_{10} ( \frac{{{\hat{θ}}_{i +}^{{f}_{1 i +}} ( 1 - {\hat{θ}}_{i +} )}^{{f}_{2 i +}}}{{{θ}_{i}^{{f}_{1 i +}} ( 1 - {θ}_{i +} )}^{{f}_{2 i +}}} ) ,\end{matrix}$$

which is also χ2(1) after adjustment by 2 ln(10)=4.605 . . . . Because the maximum likelihood estimates for the individual rows are usually different from each other, the sum of the LOD scores and the sum of the chi-squares were greater than the combined statistics. A chi-square test for heterogeneity was appropriate to test the combined estimate (Zar, 1996). Usually θ₁=0.5 is used to test linkage in familial disorders with meiotic segregation of the phenotype (Ott, 1991). In reference to sporadic cancer and especially when populations of tested cells represent sequential stages of the process with mitotic transmission of the phenotype, the null hypothesis is more appropriately verified at θ differing from 0.5. For example, a value of 0.99 is more appropriate if the marker is unchanged in the tissue, and a value of 0.01 is more appropriate for determining whether the marker has been altered from an unchanged to a changed state in the later stages of the process, i.e., invasive carcinoma. Consequently, patterns of LOD score values are used to evaluate the relationship between an altered marker and various phases of neoplasia and their progression. It has to be understood that the use of LOD scores in this analysis is not the same as that commonly used in linkage analysis of familial genetic predisposition for diseases and is intended to be used in its generic mathematical sense as likelihood tests of events. The LOD score variant of the likelihood test was used in this analysis.

### Example 5

### Superimposed Histologic and Genetic Mapping of Chromosome 9

Tumor Samples and Clinico-Pathological Data

Five cystectomy specimens containing transitional cell carcinoma (TCC) were used to create superimposed histologic and genetic maps. Fresh samples of urinary bladder tumors from 98 patients and their follow-up data were used to analyze the relationship of genetic alterations to histologic grade, invasiveness, growth pattern and to the clinical behavior of the tumor. Allelic losses in those regions of chromosome 9 that were identified as significantly altered by the superimposed histologic and genetic mapping were tested in voided urine and/or bladder washings of 26 patients with TCC. The intraurothelial precancerous changes were microscopically classified as mild, moderate, or severe dysplasia, or as carcinoma in situ. The TCCs were classified according to the three-tier histologic grading system of the World Health Organization (Koss, 1995). Their growth pattern (papillary versus nonpapillary) and depth of invasion were also recorded. The histologic sections were evaluated independently by two pathologists. DNA was extracted from mucosal samples of cystectomy specimens, individual bladder tumors and sediments of voided urine samples and/or bladder washings as previously described (Chaturvedi et al., 1997). For controls, DNA was also extracted from the peripheral blood lymphocytes and/or normal tissue in the resected specimens from each patient.

Superimposed Histologic and Genetic Maps

Cystectomy specimens were prepared as previously described (Chaturvedi et al., 1997). The inventors obtained 37, 52, 61, 42, and 39 mucosal samples respectively from each bladder. In four cases (maps 1,2,4, and 5), a single focus of grade 3, nonpapillary TCC invading the muscularis propria was present. It was accompanied by extensive precancerous lesions ranging from mild dysplasia to carcinoma in situ. In one case (map 3), multiple foci of TCC were present. One focus represented a grade 3 nonpapillary TCC with transmural invasion of the bladder wall and involvement of the perivesical adipose tissue. Two additional foci of carcinoma represented grade 3 papillary TCC without invasion. Like the other four cases, extensive areas of the urinary bladder mucosa in this case exhibited changes ranging from mild dysplasia to carcinoma in situ. The results were recorded as histologic maps. Subsequently, DNA was extracted from all mucosal samples and corresponded to microscopically verified urothelial lesions or normal bladder mucosa.

Microsatellites

A set of primers for 52 microsatellite loci on chromosome 9 based on an updated Genethon microsatellite map was purchased from Research Genetics (Huntsville, Ala.) (Gyapay et al., 1994). Several markers located within or flanking the MTS genes were also included in this example. The markers selected for testing exhibited high levels of heterozygosity and relatively uniform distribution, i.e. covered all regions of chromosome 9. The allelic patterns of markers were resolved on polyacrylmide gels after their amplification using the polymerase chain reaction as previously described (Chaturvedi et al., 1997). A minimum 50% reduction in signal intensity was required to be considered evidence of loss of heterozygosity (LOH). Tests with questionable results were repeated. In such cases the densitometric measurements were performed to ensure objective reading of the data. Testing of markers was performed in 2 phases. Initially, all 52 markers were tested on paired non-tumor versus tumor DNA samples. This revealed LOH of 15 markers which were subsequently tested on all mucosal samples to generate superimposed histologic and genetic maps.

Alterations of MTS

Allelic losses in the MTS locus were tested with marker D9S492, located between exons 1 and 2 of the MTS 1 gene. (Liu et al., 1995) Homozygous deletions within the MTS locus were tested with the following sequence-tagged site (STS) primers: 1063.7, c18.b, c5.1, RN3.1, C5.3, R2.3, R2.7, and cl.b (Kamb et al., 1994). The presence of homozygous deletions in the MTS locus as revealed by PCR using STS primers was confirmed by Southern blotting. The probes used for Southern blotting represented the DNA fragments amplified by the STS primers that exhibited homozygous deletions in a given site. The probes were labeled by the random priming method, and hybridization was carried out using standard conditions (Maniatis et al., 1989). The presence of homozygous deletions was verified by Southern blotting in five cases of bladder tumor samples and in representative tumor samples of cystectomy specimens corresponding to three foci of TCC in a cystectomy specimen used for superimposed histologic and genetic mapping of the MTS locus. The hybridizaiton signal was compared between tumor and non-tumor DNA samples.

Alterations within coding sequences of MTS 1 and 2 genes were tested by single-strand conformational polymorphism (SSCP) and direct sequencing of the PCR-amplified gene fragments using the following primers:

| | |
|---|---|
| MTS1 (exon 1) | |
| 5′ GAA GAA AGA GGA GGG GCT G 3′ | (SEQ ID NO 1) |
| 5′ GCG CTA CCT GAT TCC AAT TC 3′ | (SEQ ID NO 2) |
| MTS1 (exon 2) | |
| 5′ GGA AAT TGG AAA CTG GAA GC 3′ | (SEQ ID NO 3) |
| 5′ TCT GAG CTT TGG AAG CTC T 3′ | (SEQ ID NO 4) |
| MTS1 (exon 3) | |
| 5′ TTC TTT CTG CCC TCT GCA 3′ | (SEQ ID NO 5) |
| 5′ GCA GTT GTG GCC CTG TAG GA 3′ | (SEQ ID NO 6) |
| MTS2 (exon 1) | |
| 5′ CCA GAA GCA ATC CAG GCG CG 3′ | (SEQ ID NO 7) |
| 5′ AAT GCA CAC CTC GCC AAC G 3′ | (SEQ ID NO 8) |
| MTS2 (exon 2) | |
| 5′ TGA GTT TAA CCT GAA GGT GG 3′ | (SEQ ID NO 9) |
| 5′ GGG TGG GAA ATT GGG TAA G 3′ | (SEQ ID NO 10) |

For SSCP analysis, 100 ng of genomic DNA was amplified by PCR using 1 μM each of the primers, as previously described (Chaturvedi et al., 1997). To confirm the presence of alterations identified by SSCP, direct sequencing of PCR-generated MTS gene fragments were performed using the Sequenase PCR Product Sequencing kit (United States Biochemical Corp., Cleveland, Ohio), according to the protocol supplied by the manufacturer. All sequence modifications that represented polymorphic sites were not considered as sequence alterations and were excluded from the analysis.

In order to confirm that the structural alterations of the coding sequence of the MTS-1 gene affected the gene expression, the results of molecular analysis, i.e., LOH in p21, homozygous deletions, as well as gene mutations identified by SSCP/sequencing studies were compared with p16 expression status identified by immunohistochemistry. Staining for p16 was performed on formalin fixed paraffin-embedded tissue sections. Briefly, after hydrogen peroxide treatment to block the endogenous peroxide activity, the slides were washed in distilled water and placed in 0.01M sodium citrate buffer (pH 6.0) for 15 minutes at 95° C., which was followed by rinsing in distilled water and PBS (Phosphate buffer saline, pH 7.4). The slides were then processed for staining of p16 using the anti-p16 antibody, NCL-p16, clone DCS-50 (Vector Laboratories, Burlingame, Calif.) at a 1:25 dilution. The primary antibody was visualized using ABC Elite Kit (Vector Elite Kit; Vector Laboratories, Burlingame, Calif.) with 0.05% 3,3′-diaminobenzidine in Tris-HCl buffer containing 0.01% hydrogen peroxide and counterstained with 0.01% toluidine blue. In addition, all cut sections were kept at 4° C. prior to staining. Tumors were considered to have a normal heterogenous p16 if they expressed relatively weak nuclear staining with considerable differences in nuclear intensity, including many negative cells. A tumor was termed p16 negative if no malignant cells had positive staining and at least several contiguous p16 positive non-tumor stromal cells were present as internal controls. Each section was submitted by pathology number and the scorer did not know the status of 9p21 LOH or MTS-1 with SSCP and sequencing studies.

Identification of Chromosome 9 Allelic Losses in Voided Urine Samples

Twenty hypervariable markers corresponding to regions of chromosome 9 disclosed as significantly altered by superimposed histologic and genetic mapping studies were tested on DNA extracted from the sediments of voided urine samples and/or bladder washings of 26 patients with TCC of the bladder. The current and past clinico-pathologic data were used to evaluate the status of these patients utilizing the TNM staging system (Fleming et al., 1997). DNA extracted from sediments of voided urine samples of 10 healthy individuals with no clinical signs of urinary bladder tumors were used as controls.

Analysis of Data

For the purpose of statistical analysis the intraurothelial precancerous changes were classified into two major groups: low grade intraurothelial neoplasia (mild and moderate dysplasia; LGIN) and high grade intraurothelial neoplasia (severe dysplasia and carcinoma in situ; HGIN).

Three-dimensional displays of chromosomal alterations in relation to progression of the neoplasia from a precursor intraurothelial condition to invasive cancer were generated and initially analyzed by the nearest-neighbor analysis (Hartigan, 1975). A nearest neighbor analysis was performed on the three-dimensional stacks of maps consisting of plots of marker alterations by location on the histologic bladder maps and on chromosomal vectors. An altered region was considered a neighbor of another altered region if the two were side by side in the same marker plot or above and below each other. An altered region was also considered to be connected to another altered region if there was a continuous string of altered regions between them. Since the bladder was laid open and pinned flat, the left-most and right-most regions were also neighbors.

The relationship between altered markers and progression of urothelial neoplasia from precursor conditions to invasive carcinoma revealed by superimposed histologic and genetic mapping were tested by a modified LOD score analysis as previously described (Chaturvedi et al., 1997). Cumulative LOD scores were calculated at variable Θ (0.01, 0.5, and 0.99). Stringency level 1 designated LOD scores for specific stages of neoplasia. Stringency level 2 designated LOD scores for progression to higher stages of neoplasia. The patterns of LOD scores ≧3 at Θ=0.01 or 0.99 and LOD scores <3 at Θ=0.5 for the same marker were considered significant. The strongest association between an altered marker and neoplasia was when a LOD score was ≧3 at Θ=0.99 and 0.5 and <3 at 0=0.01. The use of LOD scores in this analysis was not the same as that commonly used in linkage analysis of familial genetic predisposition for diseases (Ott, 1991). Rather, it was intended to be used in its generic mathematical sense as a likelihood test of events (Brownlee, 1965). The LOD score variant of the likelihood test was used.

The relationship among altered markers, the MTS genes, and various clinico-pathological parameters were tested by Gehan's generalized Wilcoxon, log-rank tests, and Kaplan-Meier analysis.

Results: Superimposed Histologic and Genetic Mapping

The initial testing of paired normal and tumor DNA samples from the same patient revealed LOH in 15 out of 52 tested markers. No shortening or expansion of the repetitive regions was identified. None of the cystectomy cases used for superimposed histologic and genetic mapping showed evidence of chromosome 9 monosomy, i.e., none of the cases showed LOH of all informative markers indicating complete loss of chromosome 9. The list of tested markers, their alterations, and chromosomal location is illustrated in FIG. 1. Testing of alterations on multiple samples from the same patient revealed the same pattern of allelic loss, i.e., the same allele was always altered (lost), indicating a clonal relationship among the samples with an altered marker. The superimposition of distributions of marker alterations over the histologic maps disclosed two basic patterns of chromosome 9 deletions: scattered and plaque-like. Some of the plaque-like alterations involved large areas of urinary bladder mucosa encompassing various precursor conditions and even some areas of morphologically normal urothelium.

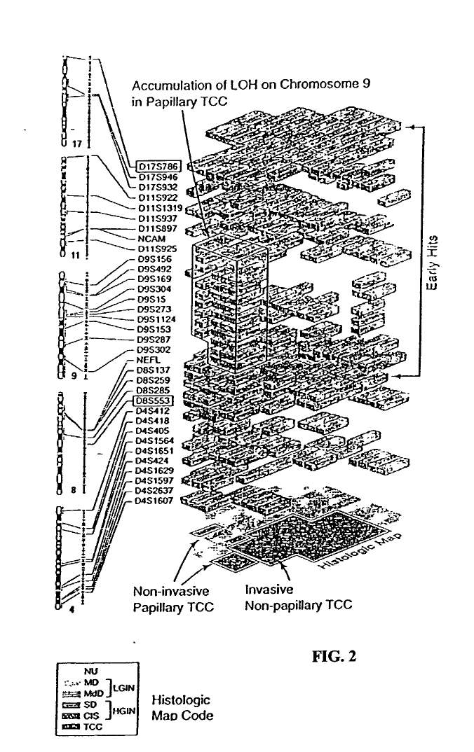

The three-dimensional superimposed histologic and genetic maps generated by the nearest neighbor analysis visualized the patterns of alterations of the entire chromosome in relation to neoplastic progression (FIG. 2). This analysis disclosed that scattered foci of alterations were in fact located within the field change in which other chromosomal regions were deleted and involved larger areas of the urinary bladder mucosa. An example of the nearest neighbor analysis in a case of multifocal TCC discloses LOH involving a large area of urinary bladder mucosa in locus D9S273 (q12-13) and a somewhat smaller area in locus D9S153 (q21). Marker D9S273 (q12-13) shows significant LOD scores in relation to all phases of neoplasia. It is evident that in this case the two separate foci of superficial papillary TCC developed in association with extensive losses of multiple markers on chromosome 9. Invasive non-papillary TCC in the same bladder did not show accumulation of multiple allelic losses of chromosome 9 and is distinct from two synchronous papillary lesions. However, both types of the lesions (superficial papillary and invasive non-papillary) have originated from the same large pre-existing field change exhibiting LOH of D9S273.

The analysis of LOD scores revealed that the markers with statistically significant relationship to the development and progression of urothelial neoplasia were located in several distinct chromosomal regions: p21-23 (D9S156); p11-13 (D9S304); q12-13 (D9S273, D9S166); q21 (D9S252); q22 (D9S287, D9S180); q34 (D9S66). Markers D9S156, D9S304, D9S166, D9S252, D9S180, and D9S66 were altered early in low grade neoplasia and also involved some adjacent areas of morphologically normal urothelium. None of the alterations could be exclusively related to the later phases of urothelial neoplasia, i.e., invasive carcinoma. Overall, the number of markers with statistically significant LOD scores did not increase with progression of intraurothelial neoplasia from low to high grade and with development of the invasive phenotype.

It appeared that a pericentromeric region on a q arm (q12-13) flanked by the markers D9S15 and D9S175 spanning approximately 4 cM represented the critical region deleted in early urothelial neoplasia. Allelic losses in this area involving markers D9S273 and D9S166 were found as significant changes of early phases of intraurothelial neoplasia in 3 of 5 cases tested by the superimposed histologic and genetic maps. The smallest deleted region in this area was restricted to 0.1 cM and was flanked by markers D9S273 and D9S1124. Additional regions on chromosome 9 potentially involved in early urothelial neoplasia are shown and defined in FIG. 12.

Superimposed Histologic and Genetic Mapping of the MTS Locus

Superimposed histologic and genetic mapping of homozygous deletions in the MTS locus was performed with STS primers in a single cystectomy specimen that on preliminary testing of normal versus tumor DNA exhibited homozygous deletions of the STS'S. In addition, the marker D9S492 (located between exon 1 and 2 of MTS 1) and the nearest flanking marker D9S169 showed LOH in this case. Homozygous deletions of STS clustered in the region corresponding to exon 2 and flanking the 5′ region of the MTS 1 gene. Early alterations involving homozygous deletions of one STS (C5.1) were mapped to morphologically normal mucosa adjacent to LGIN. Gradual expansion of the deleted region with eventual homozygous deletions of 4 adjacent STS occurred in the course of LGIN development and subsequent progression to HGIN and TCC. Moreover, the development of non-invasive papillary high grade TCC was associated with allelic loss of two adjacent hypervariable markers, D9S492 and D9S169, spanning an approximate 10-cM segment (FIG. 12). Superimposition of homozygous deletions in the MTS locus over the histologic maps disclosed that areas of urinary bladder mucosa with precursor conditions ranging from LGIN to HGIN and exhibiting progressively widening homozygous deletions in the MTS locus were adjacent to each other and formed plaque-like areas corresponding to the distribution of preneoplastic intraurothelial changes. This analysis disclosed that a relatively small focus of deletion in the MTS locus is unstable and may expand in progression of urothelial neoplasia from intraurothelial precursor conditions to TCC.

Allelic Losses of Chromosome 9 and Alterations of MTS1&2 in relation to Clinicopathological Parameters of Urinary Bladder Tumors

The chromosomal regions which were identified as significantly altered in relation to development of urothelial neoplasia by superimposed histologic and genetic mapping were tested with the use of hypervariable markers for potential allelic losses in 98 urinary bladder tumors of various histologic grades, growth patterns, invasiveness, and in relation to long-term follow-up data (Tables 5 and 6). Alterations of MTS 1 and 2 such as homozygous deletions in the MTS locus as well as structural alterations (mutations or deletions) of their coding sequences were also analyzed (Table 7). Allelic losses of six regions, i.e., p21-23, p11-13, q12-13, q21, q22, and q34 identified by superimposed histologic and genetic mapping were present in 18.3% to 67.1% of all tumors. Alterations involving only one of the above listed regions as the sole chromosome 9 allelic loss were identified in 31.5% of all tumors. The extensive allelic losses defined as involvement of three or more regions (including chromosome 9 monosomy, i.e. LOH of all informative markers tested) were present in 59.7% of all tumors. The allelic losses in the six tested regions of chromosome 9 seemed to be ubiquitous in bladder tumors and could not be related to any specific pathogenetic subsets (papillary, non-papillary) histologic grade, invasion, or clinical aggressiveness of TCC.

Allelic losses of p21-23 and homozygous deletions in the MTS locus were documented in 57.5% and 67.6% of the cases respectively. However, the mutations or large deletions directly involving the coding sequences of the MTS 1 and 2 genes were less frequent and could be documented in only 13.7% and 6.8% of the cases respectively. In addition, when the molecular data on the MTS-1 gene were related to patterns of p16 expression, it was determined that in the presence of a mutation that was that associated with LOH in the MTS-1 locus (only one mutant allele of the gene was present) no staining for p16 could be identified by immunohistochemistry. However, when a mutation within the MTS-1 gene occurred in the absence of LOH, it was associated with a normal heterogenous staining pattern indicating the presence of at least one normally functioning MTS-1 allele. These studies provided confirmation that the coding sequence alterations of the MTS-1 gene identified by SSCP and sequencing studies represent real mutations of the gene that altered its expression pattern as well as further confirming the accuracy of the molecular data.

TABLE 5
Distribution of Chromosome 9 and MTS Gene Alterations in Relation to
Pathologic Features of Transitional Cell Carcinoma*
(Analysis of 98 Cases)

| | | | | | | | | | | Alterations of MTS | |
|---|---|---|---|---|---|---|---|---|---|---|---|
| | Evidence of LOH in different regions of Chromosome 9 | | | | | | | | Homozygotic deletions | genes coding sequences*** | |
| | p21-23 | p11-13 | q12-13 | q21 | q22 | q34 | 0-2 | ≧3 | in MTS locus.** | MTS1 | MTS2 |
| Growth pattern: | | | | | | | | | | | |
| (2) papillary | 55.6 | 29.1 | 63.0 | 19.6 | 64.9 | 67.3 | 36.4 | 63.6 | 71.7 | 16.7 | 7.3 |
| (1) non-papillary | 63.2 | 15.8 | 36.8 | 14.3 | 50.0 | 71.4 | 57.9 | 42.1 | 55.6 | 5.0 | 5.3 |
| Histologic Grade: | | | | | | | | | | | |
| 1-2 | 51.3 | 25.0 | 59.0 | 18.2 | 65.1 | 66.7 | 40.0 | 60.0 | 76.9 | 15.0 | 7.5 |
| 3 | 64.7 | 26.5 | 52.9 | 18.5 | 55.9 | 70.6 | 44.1 | 55.9 | 56.2 | 11.8 | 5.9 |
| Superficial | 53.8 | 30.8 | 65.8 | 12.5 | 70.0 | 66.7 | 33.3 | 66.7 | 75.0 | 17.9 | 7.7 |
| Invasive | 60.6 | 18.2 | 48.5 | 25.0 | 52.9 | 67.6 | 48.5 | 51.5 | 60.0 | 8.8 | 5.7 |
| Total | 57.5 | 25.7 | 56.2 | 18.3 | 60.3 | 67.1 | 40.3 | 59.7 | 67.6 | 13.7 | 6.8 |

*The numbers indicate percentage of cases with alterations in a given category of tumors. The following markers were used to test allelic losses on chromosome 9: pter, D9S178; p21-23, D9S492, D9S171, D9S169, D9S270; p13, D9S52, D9S304, D9S200; q12-13, D9S273, D9S166, D9S1124, D9S175; q21, D9S167, D9S152, D9S252; q22, D9S151, D9S287, D9S180, D9S176; q34, D9S179, AB11, D9S66; qter, D9S179.
**Homozygotic deletions in the MTS locus were tested with STS primers.
***Alterations of coding sequences of the MTS genes were tested by SSCP and direct sequencing.

TABLE 6
Summary of Statistical Analysis Among Alterations of Chromosome 9 and
Clinico-Pathologic Parameters
(Analysis of 98 Cases)

| | Chromosome 9 regions | | | | | | # of chromosome 9 regions |
|---|---|---|---|---|---|---|---|
| | with evidence of LOH (p value) | | | | | | with evidence of LOH (p value) |
| Feature | p21-23 | p11-13 | q12-13 | q21 | q22 | q34 | 0-2 versus ≧3 |
| Growth pattern | 0.56 | 0.25 | 0.048 | 0.65 | 0.24 | 0.73 | 0.10 |
| Histologic grade | 0.25 | 0.89 | 0.60 | 0.97 | 0.41 | 0.72 | 0.72 |
| DNA ploidy | 0.44 | 0.50 | 0.51 | 0.47 | 0.51 | 0.10 | 0.94 |
| Invasion | 0.56 | 0.22 | 0.14 | 0.21 | 0.13 | 0.93 | 0.19 |
| Recurrence | 0.07 | 0.78 | 0.64 | 0.61 | 0.69 | 0.29 | 0.28 |
| Metastasis | 0.018 | 0.22 | 0.02 | 0.40 | 0.12 | 0.76 | * |
| Alive or Dead | 0.57 | 0.19 | 0.64 | 0.95 | 0.86 | 0.34 | 0.83 |
| Recurrence free | 0.41 | 0.70 | 0.96 | 0.93 | 0.33 | 0.10 | 0.52 |
| interval | | | | | | | |
| Metastasis free | * | 0.23 | 0.03 | 0.44 | 0.09 | 0.73 | * |
| interval | | | | | | | |
| Overall disease | 0.52 | 0.63 | 0.93 | 0.99 | 0.25 | 0.08 | 0.64 |
| free interval | | | | | | | |
| Overall survival | 0.36 | 0.26 | 0.87 | 0.64 | 0.65 | 0.38 | 0.91 |

Relationship between chromosome 9 regions with evidence of LOH and growth pattern, histologic grade, DNA ploidy, invasion, recurrence, metastiasis, and alive or dead status was analyzed by Gehan-Wilcoxon and Peto log rank tests. Recurrence, metastasis, and overall disease free intervals as well as overall survival were tested by Kaplan-Meier analysis.
* Insufficient data to perform the analysis.

TABLE 7
Summary of Sequencing Data of MTS 1 and 2 G
(Analysis of 98 Cases)

| Gene/exon | Case | Codon | Alteration | Function |
|---|---|---|---|---|
| MTS1/exon 1 | 1 | 27 | G(del) | Glu→Arg (frameshift) |
| | 2 | 4 | T(ins) | Frameshift to stop codon |
| | 3 | 24 | G(del) | Stop codon |
| MTSL/exon 2 | 4 | 148 | A(ins) | Ala→Thr |
| | 5 | 148 | G→A | Ala-Thr |
| | 6 | 113 | C→A | Leu-Met |
| | 7 | 148 | G→A | Ala-Thr |
| | 8 | 144 | G→T | No change |
| | 9 | 145 | C(ins) | Asp→Thr (frameshift) |
| | 10 | 106 | T(del) | Frameshift to stop codon |
| | 11 | 147 | G→A | Ala-Thr |
| | 12 | 53 | G→A | Met-Ile |
| | 13 | 72 | 25 nucleotide | Large deletion |
| | | | deletion | |
| MTS2/exon 1 | 14 | intron | C(ins) | No change |
| MTS2/exon 2 | 15 | 63 | G→C | Glu→Gln |

*Alterations: del, deletion; ins, insertion; G→A, G to A mutation.

TABLE 8
Clinico-Pathologic Data of Patients Whose Voided Urine and/or
Bladder Washing Samples Were Tested for Allelic Losses of
Chromosome 9
(Analysis of 26 Cases)

| Case | Current Status | | | Follow-Up | Primary Tumor | | |
|---|---|---|---|---|---|---|---|
| No | Growth | Grade | Stage | Months | Growth | Grade | Stage |
| 1 | | | T₀ | 60 | P | 2 | Tₐ |
| 2 | | | T₀ | 3 | NP | 3 | T₂ |
| 3 | | | T₀ | 15 | P | 2 | Tₐ |
| 4 | | | T₀ | 1 | NP | 3 | T₂ |
| 5 | | | T₀ | 6 | P | 2 | T₂ |
| 6 | | | T₀ | 2 | NP | 3 | Tᵢₛ |
| 7 | | | T₀ | 1 | NP | 3 | Tᵢₛ |
| 8 | | | | 0 | P | 2 | T₁ |
| 9 | P | 2 | Tₐ | 100 | P | 2 | Tₐ |
| 10 | P | 2 | Tₐ | 0 | P | 2 | Tₐ |
| 11 | P | 2 | Tₐ | 805 | P | 2 | T₁ |
| 12 | P | 2 | Tₐ | 149 | P | 2 | Tₐ |
| 13 | P | 2 | Tₐ | 140 | P | 2 | Tₐ |
| 14 | P | 2 | Tₐ | 55 | P | 1 | Tₐ |
| 15 | NP | 3 | T₁ | 2 | NP | 3 | T₁ |
| 16 | NP | 3 | T₂ | 0 | NP | 3 | T₂ |
| 17 | NP | 3 | T₂ | 3 | NP | 3 | T₂ |
| 18 | | | | 0 | NP | 3 | T₃ |
| 19 | NP | 3 | T₃ₐ | 25 | NP | 3 | T₁ |
| 20 | NP | 3 | T₃ₐ | 1 | NP | 3 | T₂ |
| 21 | NP | 3 | T_{3b} | 1 | NP | 3 | T₁ |
| 22 | NP | 3 | T₄ | 4 | NP | 3 | T₂ |
| 23 | NP | 3 | Tₐ | 120 | NP | 3 | T₃ |
| 24 | NP | 3 | Tᵢₛ | 1 | NP | 3 | T₂ |
| 25 | NP | 3 | Tᵢₛ | 1 | NP | 3 | T₁ |
| 26 | NP | 3 | Tᵢₛ | 1 | NP | 3 | T₂ |

Identification of chromosome 9 Allelic Losses in Voided Urine Samples

The clinical data of 26 patients whose urine samples were tested for LOH on chromosome 9 are summarized in Table 8. Alterations of at least one of the selected markers could be documented in 25 of 26 patients with urinary bladder carcinoma (Table 8). In the vast majority of cases, LOH of multiple markers were present. Moreover, alterations of multiple hypervariable markers were present in six of seven patients one to 60 months after the removal of grade 2-3 transitional cell carcinoma (TCC) even though disease was not clinically or microscopically detectable at that time, i.e., there was no tumor cystoscopically and urinary bladder wall biopsies as well as urine cytologies were negative for TCC and/or urothelial dysplasia at the time of testing (cases 1-7 with current status TO). Two of these patients had experienced prior recurrences of the tumor. LOH could also be identified in patients after the transurethral resection of invasive TCC with evidence of residual flat carcinoma in situ (Tis) only (cases 24-26). No allelic losses were identified in voided urine samples of 10 healthy individuals.

### Example 6

### Superimposed Histologic and Genetic Mapping of Chromosome 17

Cystectomy Specimens

Radical cystoprostatectomy specimens from four male and one female patients who had previously untreated high-grade invasive TCC of the bladder were prepared as follows. The bladder was opened longitudinally along the anterior wall and pinned down to a paraffin block. A plastic grid with holes was superimposed over the specimen and each 1×2-cm rectangle of the mucosa was individually pinned down. After the removal of the plastic grid, the entire bladder mucosa was separated into individual 1×2-cm samples and evaluated under a microscope for histologic changes on frozen sections. For microscopic evaluation of urothelium, a single histologic sections was prepared from each 1×2 cm area and was stained with hematoxylin and eosin.

DNA was extracted from each sample using a nonorganic DNA extraction kit (ONCOR). The tissue of interest was identified microscopically and initially microdissected from the frozen block. DNA was extracted from cell suspension containing approximately 90% of microscopically recognizable urothelial cells. The cell suspensions were prepared by mechanical stripping of urothelium from microdissected samples with a razor blade. Samples which contained less pure cell suspensions were not included in the analysis and are shown in histologic maps as blank areas. This procedure provided 49, 37, 61, 42 and 39 DNA samples, respectively, from each bladder. To compare the microsatellite allelic patterns, DNA was also extracted from the peripheral blood lymphocytes of each patient. The intraurothelial precancerous changes were classified as mild, moderate and severe dysplasia and carcinoma in situ. Urothelial samples classified as normal urothelium occasionally exhibited mild hyperplasia or reactive change but showed no microscopically recognizable dysplasia. The TCCs were classified according to the three-tier histologic grading system of the World Health Organization (Koss, 1995). Their growth pattern (papillary vs nonpapillary or solid) and depth of invasion were also recorded. The histologic sections were evaluated independently by two pathologists.

Microsatellites

A set of 33 microsatellite markers for the chromosome 17 loci were selected from an updated Genethon microsatellite map (Gyapay et al., 1994). Another 5 microsatellite markers that were not included on the Genethon map were also tested (Swift et al., 1995; Cropp et al., 1994). All primers were purchased from Research Genetics. The markers selected for testing exhibited high levels of heterozygosity and relatively uniform distribution, i.e., covered all regions of chromosome 17, including those of special interest in urothelial carcinogenesis. Microsatellite loci were tested by polymerase chain reaction amplification (PCR). PCR was done in a 10 μl reaction volume containing 50 ng of template DNA, 200 μM of each deoxynucleoside triphosphate, 2.5 μCi of 32P-labeled deoxycytidine triphosphate, 0.3 μM of each primer, and 0.6 U of Taq polymerase. PCR products were resolved on 6% polyacrylamide urea gel for 2 h at 55 W. Radiograms were visually examined for loss of heterozygosity (LOH). In questionable cases, densitometric measurements were performed and at least 50% of signal intensity reduction was considered as evidence of LOH.

Initially, all the microsatellite loci were tested on paired tumor and normal host DNA samples extracted from an invasive carcinoma and peripheral blood lymphocytes of the same patient. Microsatellite loci identified as altered during this initial testing were selected for superimposed histologic and genetic mapping of the entire urinary bladder mucosa. Approximately 2000 tests were performed to reveal the patterns of alterations to chromosome 17 and their relationship to the progression of urothelial neoplasia.

Superimposed Histologic and Genetic Maps

The positions of mucosal samples and their microscopic changes were recorded and displayed in the form of histologic maps. The superimposed histologic and genetic maps were generated by custom-designed software. The data consisted of a vector of chromosome 17 with microsatellite positions, their alterations, and coordinates for locations of the samples. The results were displayed by superimposed histologic and genetic maps that showed the areas of bladder mucosa with an altered microsatellite locus and its relationship to precancerous intraurothelial conditions and TCCs. In addition, the data were presented using the two-vectors technique. In this display, a vector with microsatellite positions was related to the tissue-designation vector, which showed the progression of urothelial changes from normal urothelium through dysplasia to carcinoma in situ and invasive cancer.

Superimposed Histologic and Genetic Mapping of p53

Allelic loss of p53 was tested with two markers, DS17960 and TP53. Point mutations of exons 5-9 were tested by single strand conformational polymorphism using the following primers:

| | |
|---|---|
| exon 5: | |
| 5′-TTCCTCTTCCTGCAGTACTC-3′, | (SEQ ID NO 11) |
| 5′-ACCCTGGGCAACCAGCCCTGT-3′, | (SEQ ID NO 12) |
| exon 6: | |
| 5′-ACAGGGCTGGTTGCCCAGGGT-3′, | (SEQ ID NO 13) |
| 5′-AGTTGCAAACCAGACCTAT-3′, | (SEQ ID NO 14) |
| exon 7: | |
| 5′-GTGTTGTCTCCTAGGTTGGC-3′, | (SEQ ID NO 15) |
| 5′-GTCAGAGGCAAGCAGAGGCT-3′, | (SEQ ID NO 16) |
| exon 8: | |
| 5′-TATCCTGAGTAGTGGTAATC-3′, | (SEQ ID NO 17) |
| 5′ AAGTGAATCTGAGGCATAAC-3′ and | (SEQ ID NO 18) |
| exon 9: | |
| 5′-GCAGTTATGCCTCAGATTCAC-3′, | (SEQ ID NO 19) |
| 5′ AAGACTTAGTACCTGAAGGGT-3′. | (SEQ ID NO 20) |

These sets of primers amplified 244, 184, 189, 213, and 137 bp fragments of exons 5 through 9 respectively.

Oligonucleotide primers for the single strand and conformational polymorphism were synthesized with an Applied Biosystems DNA/RNA synthesizer (model 392, Perkin Elmer Cetus) following the manufacturer's recommended procedure. Genomic DNA (100-150 ng) was amplified by PCR with 4 ng of each primer, 200 μM of each dNTP, 1 μCi of [α-32P]dCTP (Amersham; specific activity, 3000 Ci/mmol), 10 mM Tris-HCl (pH 8.3), 50 mM KCl, 1.5 mM MgCl2, 0.01% gelatin, and 1 U of Taq polymerase (Perkin Elmer Cetus) in a final volume of 10 μl. The amplification reaction consisted of 34 cycles of 1 min at 94° C., 1 min annealing at 55° C. (exons 5, 6, 7 and 9) or 58° C. (exon 8) and 2 min at 72° C. for extension. The reaction mixture was diluted (1:10) in 0.1% sodium dodecyl sulfate to 10 mM EDTA and then mixed 1:1 with a solution containing 95% formamide, 20 mM EDTA, 0.05% bromophenol blue, and 0.05% xylene cyanol. Samples were heated to 90° C. for 5 min, chilled on wet ice and resolved on a 6% polyacrylamide:Tris-borate-EDTA gel containing 10% (v:v) glycerol for 17 h at 6 W.

Initially the markers DS17960 and TP53, as well as mutations of exons 5-9 of p53, were tested on paired tumor and normal host DNA samples, extracted from a TCC and from peripheral blood lymphocytes of the same patient. Markers with alterations and exons exhibiting mutations were selected for superimposed histologic and genetic mapping. To confirm the presence of a mutation identified by single-strand conformational polymorphisms, direct sequencing of PCR-generated gene fragments were performed using the Sequenase PCR Product Sequencing kit (United States Biochemical Corp.), according to the protocol supplied by the manufacturer.

Histologic Maps

Histologic mapping of the entire urinary bladder mucosa was performed on five human cystectomy specimens with invasive transitional cell carcinoma (TCC). Four cases (maps 1, 2, 4, and 5) had a single focus of grade 3, nonpapillary TCC invading into the muscularis propria. The tumors were accompanied by extensive precancerous lesions that ranged from mild dysplasia to carcinoma in situ. In one case (map 3), multiple foci of TCC were present. One focus represented a grade 3 papillary TCC with transmural invasion of the bladder wall and involvement of the perivesical adipose tissue. Two additional foci represented grade 3 papillary TCC without invasion. Similar to the other four cases, extensive areas of the urinary bladder mucosa in this case exhibited changes that ranged from mild dysplasia to carcinoma in situ.

Superimposed Histologic and Genetic Maps

The initial testing of paired normal and tumor DNA samples from the same patient revealed alterations in 18 out of 38 tested markers on chromosome 17. The alterations consisted of loss of heterozygosity (LOH) and homozygotic deletions. No abnormally sized (shortened or expanded) alleles of repetitive sequences were identified. Testing of alterations on multiple samples from the same patient revealed the same pattern of allelic loss, i.e., the same allele was always altered (lost), indicating that a clonal relationship existed among the samples with the altered marker. The superimposition of microsatellite alterations over the histologic maps disclosed two basic patterns of chromosome 17 deletions: scattered (in a form of several isolated foci) and plaque-like. Some of the plaque-like alterations involved large areas of urinary bladder mucosa with various precursor conditions, including low-grade intraurothelial neoplasia, and some areas of morphologically normal urothelium. These findings indicated that the alteration occurred early in the process of urothelial neoplasia. Alterations of some other markers were restricted to specific stages of neoplasia, e.g., invasive carcinoma or invasive carcinoma with adjacent carcinoma in situ, which indicated an association with late phases of the process and invasive growth. Each case had a distinct pattern of chromosome 17 alterations. The three separate foci of TCCs in map 3 also showed distinct patterns of microsatellite alterations.

Superimposed Histologic and Genetic Mapping of p53

Table 9 is a summary of the p53 alteration identified in 5 cystectomy specimens. Allelic losses of the TP53 marker located within the p53 gene and of adjacent microsatellite D17S960 were identified in one case (map 5). Mutations of exons 6, 7, and 9 were present in three cases (maps 3, 4 and 5, respectively). In one case (map 5), both the mutation of exon 6 and the allelic deletions of TP53 and D17S960 were found. In two cases (maps 3 and 4), the mutation of the gene was not associated with its allelic loss. In the remaining two cases (maps 1 and 2), no alterations of p53 could be documented. Superimposed histologic and genetic mapping revealed plaque-like alterations of p53 mutations or allelic losses in the three cases. The alterations involved invasive carcinoma and large areas of urinary bladder mucosa with intraurothelial precursor conditions. The data indicated that, in all three cases, p53 alterations could be mapped to early stages of intraurothelial neoplasia consistent with low-grade intraurothelial neoplasia. In map 5, allelic loss and exon 6 mutation involved almost the entire urinary bladder mucosa, which exhibited various (low- and high-grade) intraurothelial precursor conditions. These findings indicated that both types of alterations (i.e., mutations and allelic loss) occurred early in the carcinogenesis process. Moreover, separate foci of TCC in map 3 exhibited the same mutation of p53 that was also present in the areas of intraurothelial precursor conditions involving the bladder mucosa among the tumors.

Data Analysis

Chi-square or ROC analysis revealed that the alterations of four markers (D17S849, D17S786, D17S933 and D17S807) and mutations of p53 could be related to the development and progression of urothelial neoplasia. In reference to several markers, the ROC area below the ROC curves could not be calculated. In addition, several chi-square analyses were performed using contingency tables, with a marginal number of samples required to obtain meaningful calculations. Both chi-square and ROC analyses most likely underestimated the involvement of chromosome 17 markers in urothelial neoplasia and thus did not seem to be proper methods with which to analyze this type of data (Table 10).

TABLE 9
Summary of p53 alterations in cystectomy specimens

| | Allelic loss | | p53 mutations | | | |
|---|---|---|---|---|---|---|
| Map | TP53 | D17S960 | Exon | Codon | Mutation | Function |
| 1 | RH | NI | — | — | — | — |
| 2 | RH | NI | — | — | — | — |
| 3 | RH | NI | exon 6 | 213 | G→A | Arg→Gln |
| 4 | RH | NI | exon 7 | 247 | A→G | Asn→Ser |
| 5 | LOH | LOH | exon 6 | 197 | G→A | Val→Met |

RH, retention of heterozygosity; NI, non informative; LOH, loss of heterozygosity.

TABLE 10
Alterations of chromosome 17 markers and
their relationship to urothelial neoplasia (chi-square and ROC analyses)

| | | Chi-Square | | | | | | | |
|---|---|---|---|---|---|---|---|---|---|
| Marker alteration | Type | Map 1 | Map 2 | Map 3 | Map 4 | Map 5 | Overall | Overall | ROC |
| D17S578 | LOH | | | | | | 0.36951 | 0.36951 | 0.42 |
| D17S849 | LOH | | | | | 0.02363 | | 0.02363 | D |
| D17S796 | LOH | | 0.90178 | | | | | 0.90178 | D |
| mutp53 | mut | | | 0.0001 | 0.0001 | 0.0001 | 0.0001 | 0.0001 | |
| TP53 | LOH | | | | | | 0.384615 | 0.384615 | 0.6 |
| D17S960 | LOH | | | | | | 0.307692 | 0.307962 | 0.35 |
| D17S786 | LOH | | 0.0862 | 0.58068 | | | | 0.00633 | 0.0007 |
| D17S799 | LOH | 0.72754 | | | | | 0.74386 | 0.36715 | 0.869 |
| D17S947 | LOH | | | | | | 0.34106 | 0.34106 | D |
| D17S925 | LOH | | 0.02369 | | | | | 0.02369 | 0.46 |
| D17S579 | HD | 0.576 | | | | | | 0.576 | 0.0977 |
| D17S933 | LOH | | | | 0.00153 | | | 0.00153 | 0.0002 |
| D17S932 | HD | 0.93584 | | 0.67986 | | | | 0.91074 | 0.5 |
| D17S934 | LOH | | | | | | 0.30227 | 0.30227 | D |
| D17S943 | LOH | 0.34462 | | | | | 0.16208 | 0.79546 | D |
| D17S808 | LOH | | | | | | 0.45254 | 0.45254 | D |
| D17S807 | LOH | 0.48705 | 0.12923 | | 0.0373 | | | 0.01455 | 0.0015 |
| D17S937 | LOH | | | | | | 0.0181 | 0.0181 | D |
| D17S784 | LOH | | | | | | 0.60429 | 0.60429 | 0.798 |

ROC, receiver-operating characteristic; LOH, loss of heterozygosity; HD, homozygotic deletion; mut, mutation of coding sequece; D, insufficient data to calculate an area below ROC curve.

LOD scores provided more detailed analysis of chromosome 17 alterations. The markers with statistically significant patterns of LOD scores could be related to several distinct regions of chromosome 17: p12-13 (TP53, D17S960, D17S786, D17S799 and D17S947), q21-11 (D17S579, D17S932 and D17S934), q22 (D17S943) and q24-25 (D17S807 and D17S784). Alterations of markers D17S786, D17S799, D17S947, D17S579, D17S932, D17S943 and D17S807 represented the earliest detectable changes to chromosome 17 and mapped to low-grade urothelial neoplasia and adjacent areas of microscopically normal urothelium. At least three distinct regions on chromosome 17 seemed to be consistently involved—in multistep fashion—in urothelial neoplasia. Within these regions, the number of altered markers with significant LOD score patterns increased as neoplasia progressed to high-grade intraurothelial neoplasia. However, in the chromosomal regions q12-13 and q21-11, the number of altered markers with statistically significant LOD scores decreased in foci of TCC compared with areas of high-grade intraurothelial neoplasia. This decrease could be artificial because significantly fewer TCC samples were available for calculations compared with the number of samples of preneoplastic conditions. On the other hand, if this result is accurate, then additional deletions of chromosome 17 do not play a major role in the development of invasive phenotypes in tested cases. Allelic losses and mutations of p53 were mapped to early stages (low-grades) of urothelial neoplasia. Statistically significant LOD scores for allelic losses and mutations of p53 were obtained in both levels of stringency for low- and high-grade intraurothelial neoplasia.

### Example 7

### Gentic Modeling of Human Urinary Bladder Carcinogenesis

Cystectomy Specimens

Radical cystectomy specimens from five patients who had previously untreated sporadic high-grade invasive TCC3 of the bladder were used. All patients were males and their age ranged from 47 to 78 (mean=66.4±11.9 S.D.). None of the tumors occurred in a clinical setting of a known cancer predisposing syndrome. The bladders were opened longitudinally along the anterior wall and pinned down to a paraffin block. The entire mucosa was then divided into 1×2 cm rectangular samples and evaluated microscopically on frozen sections. The tissue of interest was microdissected from the frozen block and used for DNA extraction. This procedure provided 49, 39, 65, 42 and 39 DNA samples from each bladder that corresponded to microscopically identified intraurothelial precursor lesions and invasive cancer. As a control, DNA was also extracted from the peripheral blood lymphocytes and/or normal tissue in the resected specimens from each patient. The intraurothelial precancerous changes were microscopically classified as mild, moderate, and severe dysplasia and carcinoma in situ. For statistical analysis, the precursor conditions were divided into two major categories: mild to moderate dysplasia, LGIN3 and severe dysplasia to carcinoma in situ, HGIN3. The TCC were classified according to the three-tier histologic grading system of the World Health Organization. The growth pattern of papillary versus nonpapillary or solid tumors and the depth of invasion were also recorded. In four of five cystectomy specimens, a single focus of grade 3 nonpapillary TCC invading the muscularis propria was present and was accompanied by extensive precancerous lesions ranging from mild dysplasia to carcinoma in situ. In the remaining case, multiple foci of TCC were present. One focus represented a grade 3 nonpapillary TCC with transurothelial invasion of the bladder wall and involvement of perivesical adipose tissue. Two additional foci of carcinoma represented grade 3 papillary TCC without invasion. Like the other four cases, this case exhibited changes ranging from mild dysplasia to carcinoma in situ involving extensive areas of the urinary bladder mucosa. The results of microscopic evaluation of individual mucosal samples were recorded and stored in a computer database as histologic maps.

Superimposed Histologic and Genetic Maps

The hypervariable markers were selected and used as described above. In brief, a set of primers (Research Genetics, Huntsville, Ala., USA) mapped to chromosomes 4, 8, 9, 11, and 17 was selected using an updated Genenthon microsatellite map. The allelic patterns of markers were resolved on polyacrylamide gel after their amplification using the polymerase chain reaction. A minimum of 50% reduction in signal intensity documented by densitometric measurements was required to be considered evidence of LOH 3. Testing was performed in two phases. Initially, all markers were analyzed on paired, nontumor versus invasive tumor DNA samples. The markers with evidence of- LOH were subsequently used on all mucosal samples to generate superimposed histologic and genetic maps.

Analysis of Data

The results of testing with hypervariable markers were entered into the data files and superimposed over the histologic maps. Initial raw data consisted of chromosomal vectors with a list of ordered markers, their alterations and coordinates for locations of mucosal samples, which could be used to plot their relation to microscopically classified urothelial changes. Superimposing plots of genetic changes over the histologic map provided an analysis of which areas of bladder mucosa had altered markers and whether they had a relationship to intraurothelial precursor conditions and TCC.

Three-dimensional displays of chromosomal alterations in relation to the progression of neoplasia from precursor intraurothelial conditions to invasive cancer were generated and initially analyzed by the nearest-neighbor algorithm. The relationship between altered markers and the progression of urothelial neoplasia from precursor conditions to invasive cancer revealed by superimposed histologic and genetic mapping were tested by a modified LOD score analysis. Cumulated LOD scores were calculated at variable Θ=0.01, 0.05, and 0.09. A pattern of LOD scores ≧3 at Θ=0.01 or 0.09 and LOD scores <3 at Θ=0.5 for the same marker was considered significant. By assembling the data from individual chromosomes, the genetic model of multistep carcinogenesis was generated which also includes data on chromosomes 9 and 17. Overall, nearly 8000 tests were performed to generate the genetic model of urinary bladder cancer progression and 97% of the performed tests were successful. Of 225 tested markers, 79% were informative. The summary of raw data used for assembly of the genetic model is provided in Table 11.

TABLE 11
Raw data used for assembly of genetic model of human urinary bladder
carcinogenesis

| Samples tested: | |
|---|---|
| N U3 | 53 |
| LG I N | 82 |
| HGIN | 50 |
| TCC | 49 |
| TOTAL | 234 |
| Chromosome markers tested | |
| Chromosome 4 | 45 |
| Chromosome 8 | 43 |
| Chromosome 9 | 52 |
| Chromosome 11 | 47 |
| Chromosome 17 | 38 |
| TOTAL | 225 |
| First screening of paired | |
| normal and tumor DNA | |
| Chromosome 4 | 630 |
| Chromosome 8 | 602 |
| Chromosome 9 | 728 |
| Chromosome 11 | 658 |
| Chromosome 17 | 532 |
| TOTAL | 3150 |
| Secondary screening of | |
| all mucosal samples | |
| Chromosome 4 | 900 |
| Chromosome 8 | 565 |
| Chromosome 9 | 1012 |
| Chromosome 11 1163 | |
| Chromosome 17 1152 | |
| TOTAL | 4792 |

The initial testing of paired normal and tumor DNA samples from the same patients revealed LOH in 72 of 225 tested markers. Seven markers showed expansion or shortening of their repetitive sequences that involved only individual mucosal samples and could not be statistically related to the development and progression of urothelial neoplasia. The differences in length of the repetitive sequences identified were considered as sporadic, random events not related to overall genomic instability associated with the malfunctioning DNA repair genes such as MSH2. Therefore, shortening or expansion of the repetitive sequences was not included in the final analysis of data.

Multiple mucosal samples of an individual cystectomy specimen always showed LOH of the same allele, indicating their clonal relationship. Testing of altered markers with LOH in all mucosal samples and the superimposition of their alterations over the histologic maps disclosed two basic distribution patterns of LOH: scattered (in the form of several isolated foci) and plaque-like alterations. Some of the plaque-like LOH involved large areas of urinary bladder mucosa comprising variable precursor conditions including LGIN3 and some areas of adjacent microscopically normal urothelium. Such findings indicated that the LOH occurred early in the process of urothelial neoplasia for these markers, e.g., D9S273 and D4S9548 B. At the other end of the spectrum were markers with LOH restricted to specific stages of neoplasia, e.g. invasive carcinoma or invasive carcinoma with adjacent carcinoma in situ, indicating their involvement in the late phases of the process and possibly invasive growth, e.g., D9S1924 and D17S849.

Nearest neighbor analysis confirmed a clonal relationship between populations of urothelial cells exhibiting LOH. During the assembly of the three-dimensional models depicting the distribution of chromosomal allelic losses, none of the mucosal areas with LOH were rejected by the nearest neighbor algorithm. Even those markers that showed scattered foci of LOH were in fact located within larger areas exhibiting LOH in other loci, i.e., represented secondary alterations within the pre-existing abnormal clone.

Each of the tested chromosomes exhibited a distinct pattern of LOH and none of the markers with LOH was altered in every cystectomy specimen. The markers with statistically significant LOD scores linking their LOH to various phases of urothelial neoplasia were located in several distinctive regions of each chromosome (FIG. 7, FIG. 11, FIG. 12, FIG. 14, and FIG. 20). These regions identified the locations of putative tumor suppressor gene loci potentially playing a role in the development and progression of urothelial neoplasia. They are shown on individual chromosomal vectors as minimal deleted areas and are defined by their flanking markers and a presumptive length of deleted segments in cMs.

By assembling the data from individual chromosomes, a model of multistep urinary bladder carcinogenesis was produced (FIG. 2). This model shows the evolution of LOH in individual loci and their significance for the development and progression of urothelial neoplasia as revealed by LOD scores. Of 72 markers with LOH, 47 showed a statistically significant relationship to urothelial neoplasia. The markers with significant LOD scores linking their allelic loss to different phases of urothelial neoplasia clustered in 33 distinct chromosomal regions, identifying these regions as positions of putative tumor suppressor gene loci that may potentially play a role in the development of urinary bladder cancer.

It is evident that the vast majority of allelic losses occurred in the early phases of urothelial neoplasia (LGIN) and often involved the adjacent urothelium in which there were no microscopically recognizable changes. Overall, 33 (70%) of the markers exhibited statistically significant LOH in association with the development of precursor intraurothelial conditions, whereas 14 (30%) of the alterations were more likely related to the development of the invasive phenotype. Interestingly, 21 (45%) of statistically significant LOH could be identified in morphologically normal urothelium antecedent to the development of microscopically recognizable precursor lesions.

### Example 8

### Superimposed Histologic and Genetic Mapping of Chromosome 3

Tumor Samples

Eight cystectomy specimens of previously untreated patients containing invasive transitional cell carcinoma were used to create superimposed histologic and genetic maps. Hypervariable DNA markers mapped to the two putative tumor suppressor gene.

5 loci located within 3p21.3 and 3q21-23 regions were subsequently tested on 32 urinary bladder tumor samples and on voided urine samples of 22 patients with urinary bladder cancer. The histologic sections were evaluated independently by two pathologists. Transitional cell carcinomas were classified according to the three-tier histologic grading system of the World Health Organization system. Their growth patterns (papillary vs. non-papillary) and depth of invasion were also recorded.

Superimposed Histologic and Genetic Maps

Radical cystectomy specimens were prepared as described previously. In brief, each bladder was opened longitudinally along the anterior wall and the entire mucosa was divided into 1×2 cm mucosal samples. The status of urothelium and the intraurothelial precursor conditions were classified on frozen sections as mild, moderate or severe dysplasia, carcinoma in situ, or TCC. The inventors obtained 37, 52, 61, 42, 39, 29, 33, and 44 mucosal samples respectively from each bladder. In seven cases, a single focus of grade 3 non-papillary TCC invading the muscularis propria was present. In each case, a focus of invasive cancer was accompanied by extensive precancerous lesions ranging from mild dysplasia to carcinoma in situ. In one remaining map (map 3) multiple foci of TCC were present. One focus represented a grade III non-papillary TCC with transmural invasion of the bladder wall and involvement of the perivesical adipose tissue. Two additional foci of carcinoma represented grade III papillary TCC without invasion. Similar to other cases, extensive areas with precursor intraurothelial conditions ranging from mild dysplasia to carcinoma in situ were present in the adjacent mucosa.

For superimposed histologic and genetic mapping, DNA was extracted from all individual mucosal samples and corresponded to microscopically identified precursor intraurothelial conditions and invasive TCC. DNA was extracted from cell suspensions containing approximately 90% of microscopically recognizable urothelial cells. The cell suspensions were prepared by mechanical stripping of urothelium from microdissected samples with a razor blade. Samples containing less pure cell suspension were not included in the analysis and are shown in the histologic maps as blank areas. For control purposes, DNA was also extracted from peripheral blood lymphocytes, and/or normal tissue in resection specimens of each patient.

Microsatellites

A set of 36 microsatellite markers for the chromosome 3 loci were selected from an updated Genethon microsatellite map (Gyapay et al., 1994). All primers were purchased from Research Genetics. The markers selected for testing exhibited high levels of heterozygosity and relatively uniform distribution, i.e., covered all regions of chromosome 3, including those of special interest in urothelial carcinogenesis. The allelic patterns of markers were resolved on polyacrylamide gels after their amplification using the polymerase chain reaction as previously described (Chaturvedi et al., 1997). Radiograms were visually examined for loss of heterozygosity (LOH). In questionable cases, densitometric measurements were performed and at least 50% of signal intensity reduction was considered as evidence of LOH. Initially, all the microsatellite loci were tested on paired tumor and normal host DNA samples extracted from an invasive carcinoma and peripheral blood lymphocytes of the same patient. Microsatellite loci identified as altered during the initial testing were selected for superimposed histologic and genetic mapping of the entire urinary bladder mucosa. Approximately 2000 tests were performed to reveal the patterns of alterations to chromosomes 3 and their relationship to the progression of urothelial neoplasia.

Assembly and Analysis of Data

The positions of mucosal samples and their microscopic changes were recorded and displayed in the form of histologic maps. The superimposed histologic and genetic maps were generated by custom-designed software. The data consisted of a vector of chromosome 3 with microsatellite positions, their alterations, and coordinates for locations of the samples. The results were displayed by superimposed histologic and genetic maps that showed the areas of bladder mucosa with an altered microsatellite locus and its relationship to precancerous intraurothelial conditions and TCC's.

Three-dimensional displays of chromosomal alterations in relation to progression of neoplasia from precursor intraurothelial conditions to invasive cancer were generated and initially analyzed by the nearest neighbor analysis as described above. The relationship between altered markers and progression of urothelial neoplasia from precursor conditions to invasive carcinoma were tested by a modified a LOD score analysis. In brief, cumulative LOD scores were calculated at variable Θ (0.01, 0.5, and 0.99). Stringency level 1 designated LOD scores for specific stages of neoplasia. Stringency level 2 designated LOD scores for progression to higher stages of neoplasia. The patterns of LOD scores ≧3 at Θ=0.01 or 0.99 and LOD scores <3 at Θ=0.5 for the same marker wereconsidered significant. The use of LOD scores in this analysis were not the same as that commonly used in linkage analysis of familiar genetic positions for diseases. Rather it was intended to be used in genetic mathematical sense as likelihood test of events. The relationship among altered markers and various clinicopathologic parameters of TCC's were tested chi-square statistics. Results are summarized in Table 12.

TABLE 12
ALTERATIONS IN 3p21 AND 3q21-25 LOCI BY
CLINICOPATHOLOGICAL PARAMETERS OF UROTHELIAL CARCINOMA
Putative Tumor Suppressor Gene Loci

| | p21(D3S1277 - D3S1100) | | q21-25(D3S1541 - D3S1512) | |
|---|---|---|---|---|
| | | Significance | | Significance |
| | Frequency(%) | (P value) | Frequency(%) | (P value) |
| Growth Pattern | | | | |
| Papillary | 30% | 0.27 | 50% | 0.10 |
| Non-papillary | 17% | | 21% | |
| Histologic grade | | | | |
| Low-grade (1-2) | 20.8% | 0.51 | 28.6% | 0.54 |
| High-grade (3) | 28.6% | | 36% | |
| Stage | | | | |
| Superficial | | | | |
| (Tₐ₋₁, T₁ₛ) | 40% | 0.13 | 50% | |
| Advanced | 14.3% | | 27.3% | 0.20 |
| (T₂₋₄) | | | | |
| Total | 22.6% | | 34.4% | |

Results: Superimposed Histologic and Genetic Mapping

The initial testing of paired normal and tumor DNA samples from the same patient revealed loss of heterozygosity in 10 out of 33 tested markers on chromosome 3. Testing of alterations on multiple samples from the same patient revealed the same pattern of allelic loss, i.e. the same allele was always altered, indicating the clonal relationship among the samples with altered markers. The superimposition of microsatellite alterations over the histologic maps disclosed two basic patterns of chromosome 3 deletions: scattered (in the form of several isolated foci) and plaque-like. Some of the plaque-like alterations involved large areas of urinary bladder mucosa with various precursor conditions, including low-grade intraurothelial hyperplasia, and some areas of morphologically normal mucosal urothelium. These findings indicated that the alterations occurred early in the process of urothelial neoplasia and are associated with clonal expansion of abnormal urothelial cells involving large areas of urinary bladder mucosa.

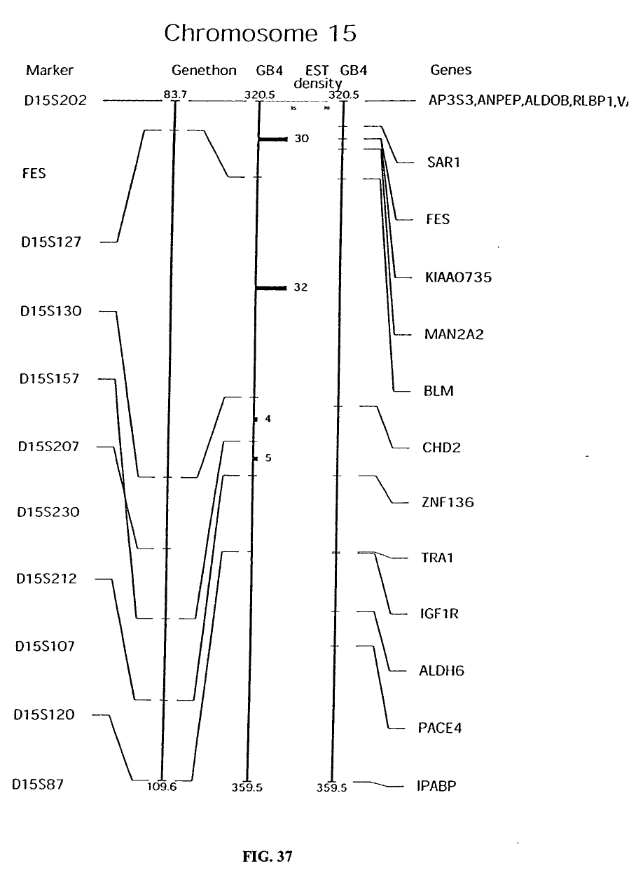

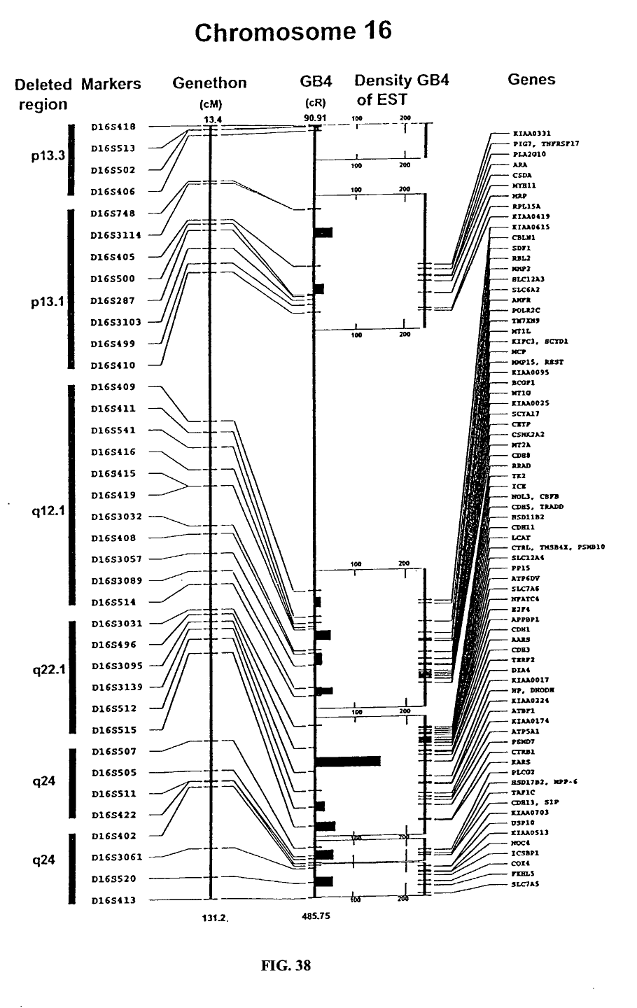

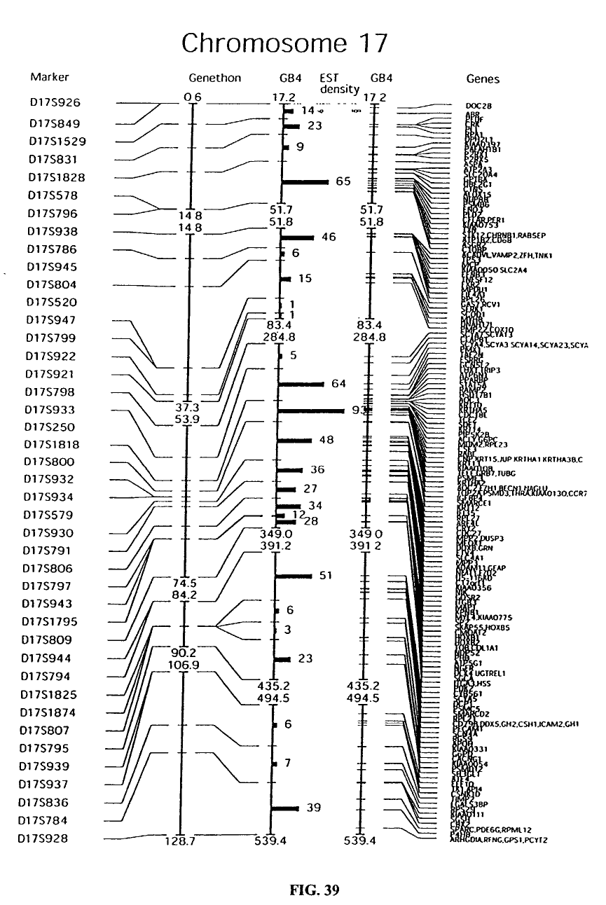

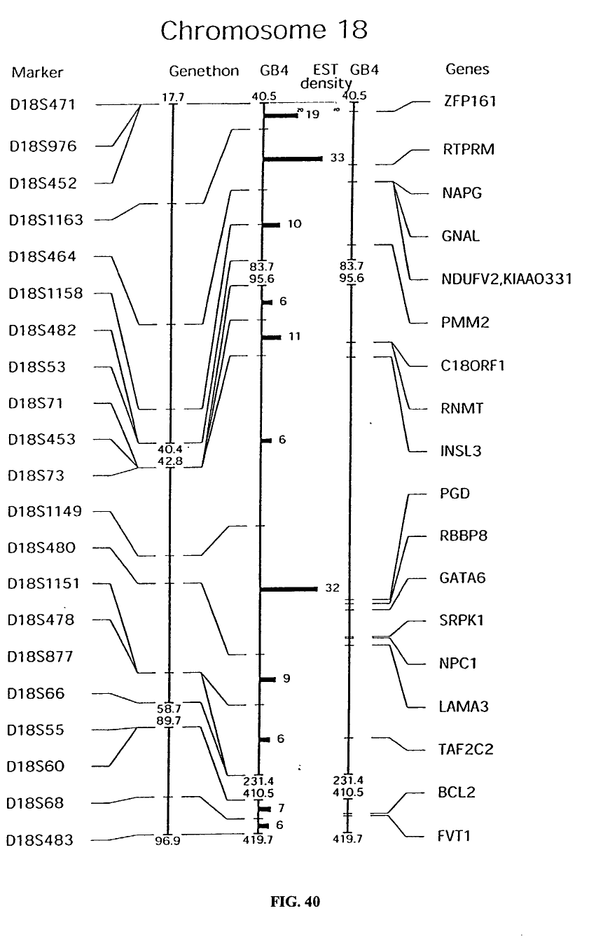

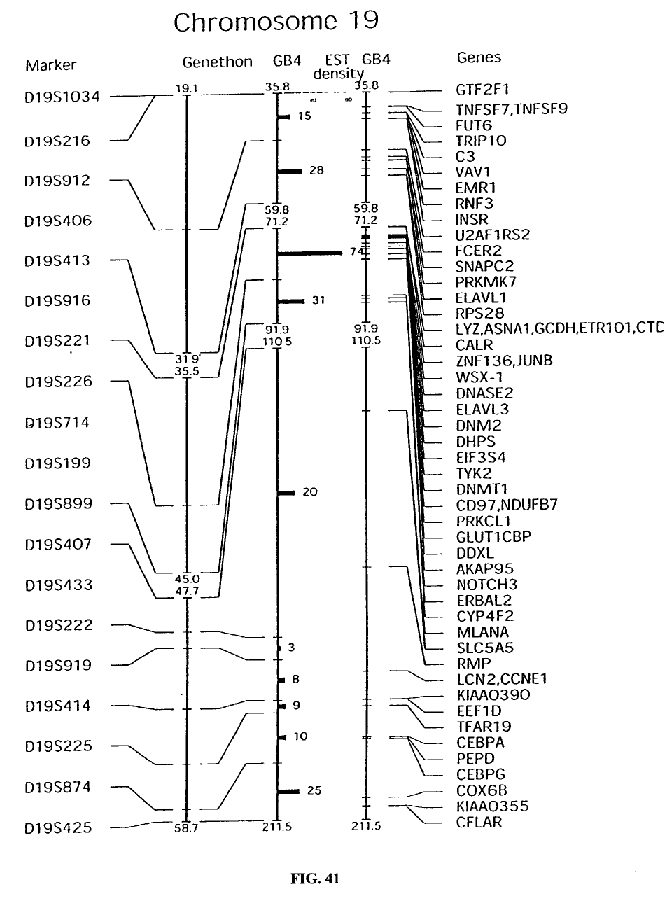

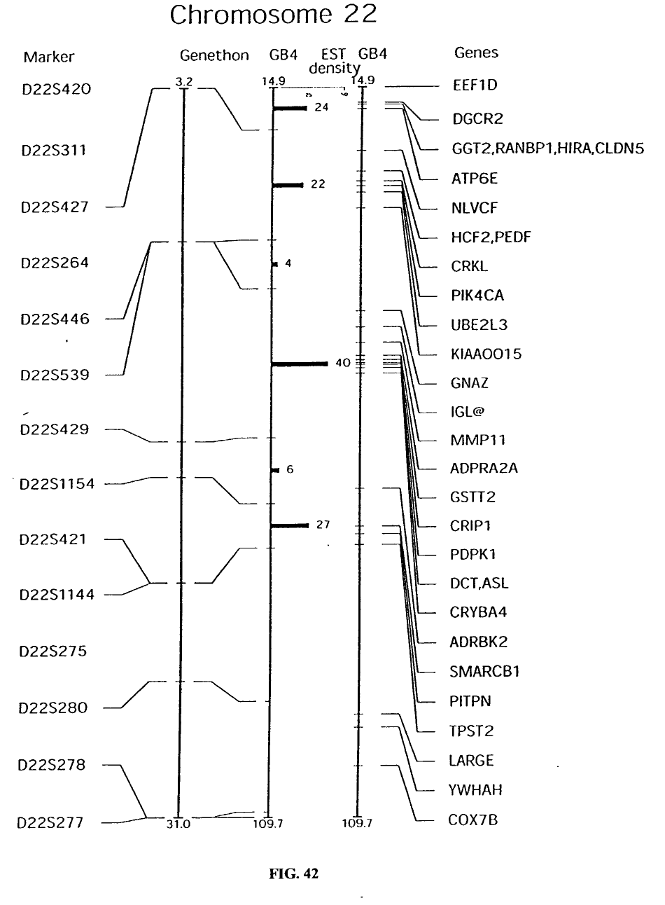

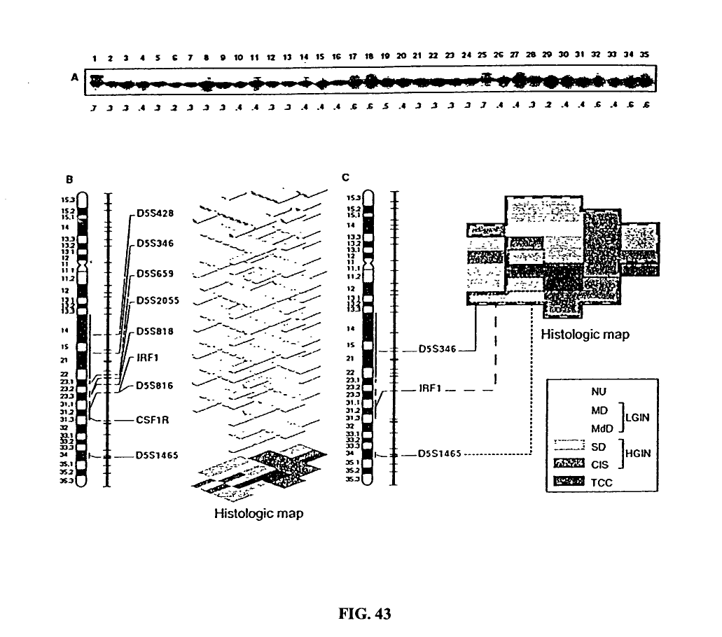

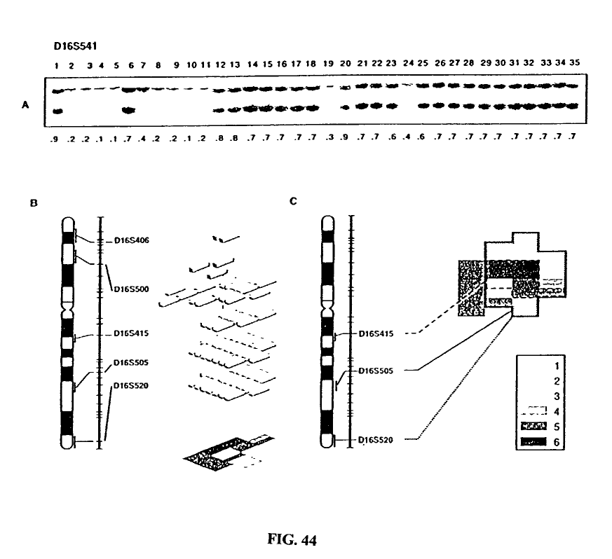

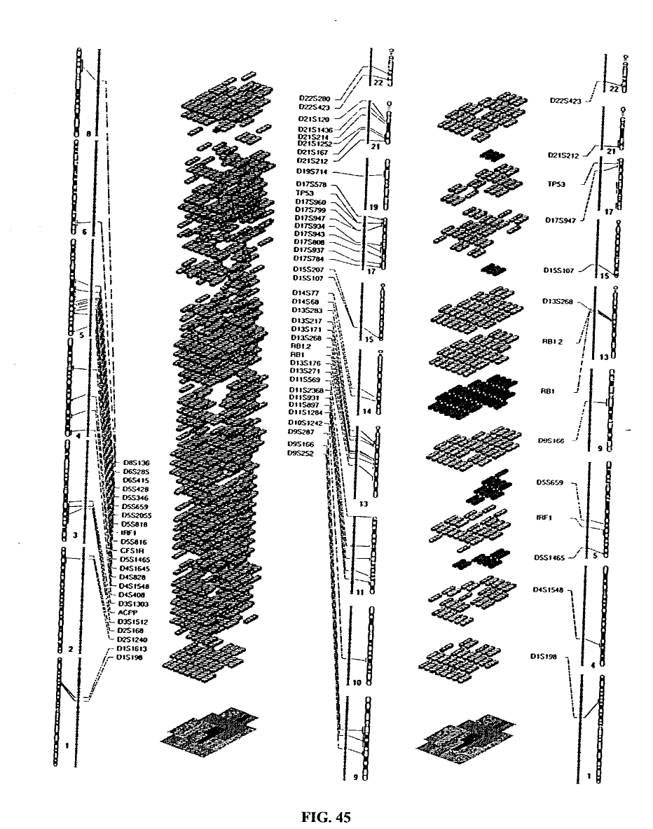

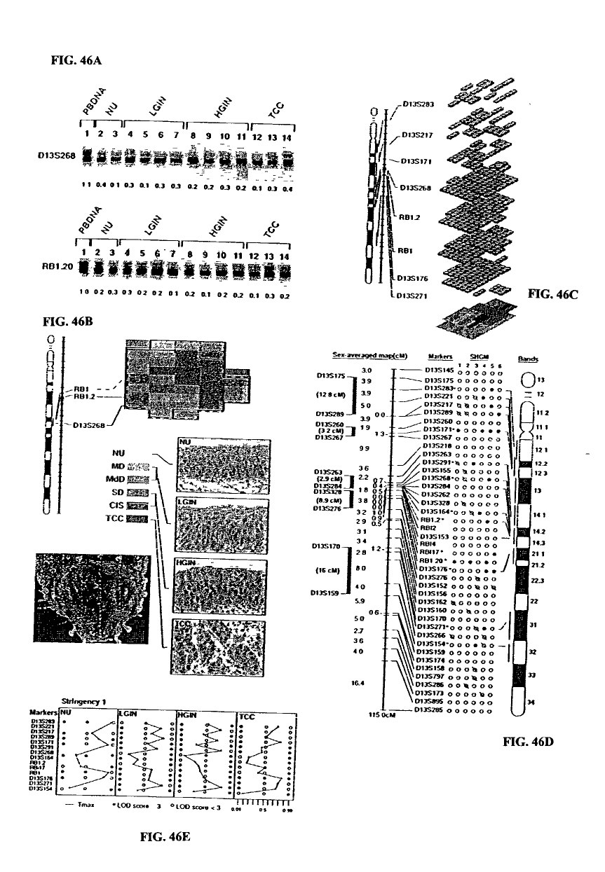

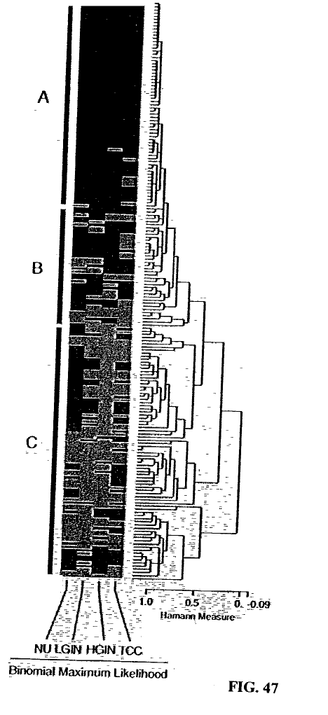

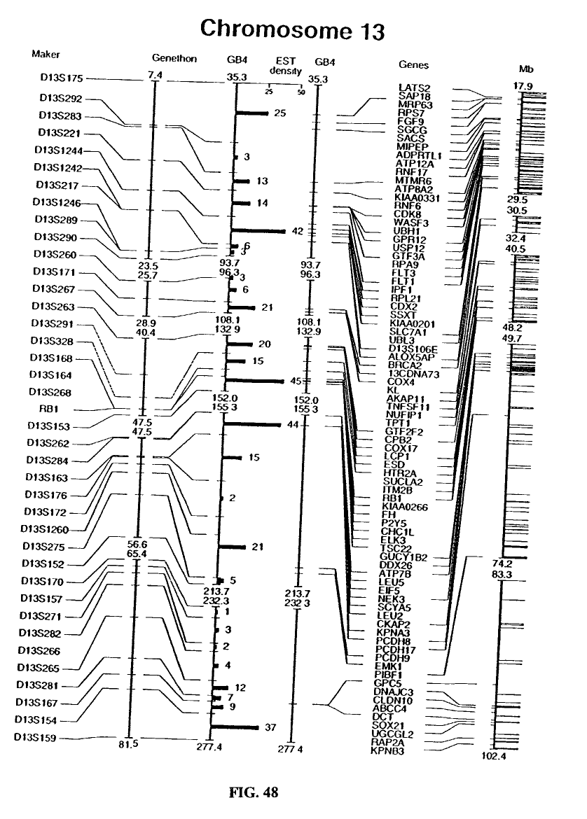

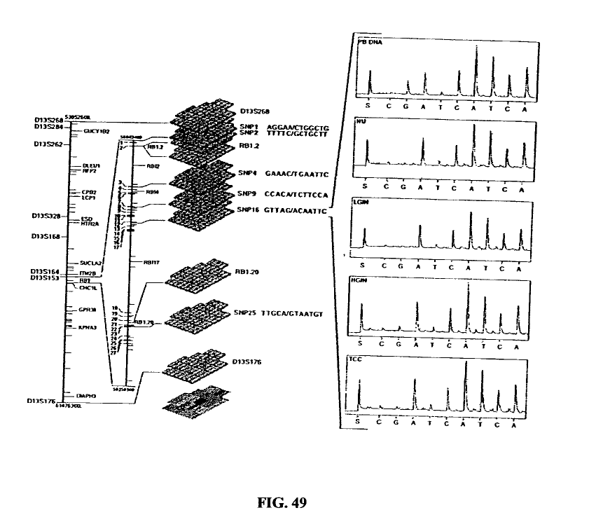

Alterations of some markers were restricted to specific stages of neoplasia, e.g. invasive carcinoma or invasive carcinoma with adjacent high-grade intraurothelial neoplasia, indicating that their alterations were associated with the late phases of the process and possibly with invasive growth. The three-dimensional patterns of allelic losses on chromosome 3 in individual cases were assembled by the nearest neighbor analysis. This disclosed that even those markers which showed scattered foci of alterations were in fact located within the field changes in which other chromosomal regions showed larger areas of involvement. The markers exhibiting LOH were clustered in 4 distinct regions of chromosome 3: 3p21 (D3S1298), 3q13.3 (D3S1278, D3S1303), 3q21-23 (D3S1541, ACPP, D3S1512), 3q26-28 (D3S1246, D3S1754, D3S1262, D3S1661). The LOD score analysis revealed that an 11 cM segment flanked by D3S1541 and D3S1512 centered around the ACPP marker represented a critical deleted region mapped to 3q21-23 involved in the clonal expansion of urothelial cells preceding the development of microscopically recognizable intraurothelial precursor changes (FIG. 4). Allelic losses in this area were identified in 4 out of 8 tested cystectomy specimens implicating its frequent involvement in urinary bladder neoplasia. Expansion of losses on the q arm ultimately involving a large segment spanning the 3q13-28 regions and flanked by the markers D3S1278 and D3S1661 was associated with the development of high-grade intraurothelial neoplasia and progression to invasive disease. This expansion was seen, however, in one out of eight cystectomy cases only.

The LOD score analysis of allelic losses on the p arm identified within the p21 region revealed a 9.4 cM deleted segment flanked by markers D3S1277 and D3S1100 centered around the marker D3S9298. The allelic losses in this area exhibited statistically significant LOD scores in association with the development of invasive cancer, but were identified in one out of.eight cystectomy cases only.

Testing of Allelic Losses on Chromosome 3 in Bladder Tumor and Voided Urine Samples

The summary of data on allelic losses of chromosome 3 tested with 17 hypervariable markers on voided urine and urinary bladder tumor samples is provided in FIG. 26. The 17 hypervariable markers selected for this analysis were mapped to chromosome 3 regions that exhibited allelic losses identified by our superimposed histologic and genetic mapping studies. In addition, the two nearest markers flanking the deleted segment of the chromosome were tested. It is evident that the alterations on both arms of chromosome 3 occurring most frequently in the form of allelic losses and occasionally showing expansion or shortening of repetitive sequences could be identified in the vast majority of voided urine and bladder tumor samples. The allelic losses in the q21-23 regions formed a clearly defined locus centered around the ACPP marker and flanked by D3ST541 and D3S1592 microsatellites.

The allelic losses in this region could be identified in approximately 35% of informative bladder tumor samples and in more than 50% of informative voided urine samples obtained from patients with TCC. Moreover, allelic losses in the ACPP locus could be identified in four of five informative patients with a history of TCC only and no evidence of tumor at the time of testing. The alterations in the remaining portions of the chromosome did not form the clearly defined region and most likely represented random, scattered events. Moreover, the allelic losses in the putative tumor suppressor gene locus in the p21 region identified by superimposed histologic and genetic mapping could be identified in only 12% of bladder tumor samples. Similarly, the losses in this area could be identified in 10% of the voided urine samples. In summary, testing of chromosome 3 allelic losses on multiple voided urine and tumor samples confirmed the presence of a well-defined putative tumor suppressor gene locus in the q21-23 region in the vicinity of the ACPP marker.

### Example 9

### Superimposed Histologic and Genetic Mapping of Chromosome 13 Cystectomy Specimens

Radical cystectomy specimens from five patients who had previously untreated sporadic high-grade invasive transitional cell carcinoma (TCC) of the bladder were used. All patients were males and their age ranged from 47 to 78 (mean=66.4±11.9 S.D.). None of the tumors occurred in a clinical setting of a known cancer predisposing syndrome. The bladders were opened longitudinally along the anterior wall and pinned down to a paraffin block. The entire mucosa was then divided into 1×2 cm rectangular samples and evaluated microscopically on frozen sections. The tissue of interest was microdissected from the frozen block and used for DNA extraction.

This procedure provided 49, 39, 65, 42 and 39 DNA samples from each bladder that corresponded to microscopically identified intraurothelial precursor lesions and invasive cancer. As a control, DNA was also extracted from the peripheral blood lymphocytes and/or normal tissue in the resected specimens from each patient. The intraurothelial precancerous changes were microscopically classified as mild, moderate, and severe dysplasia and carcinoma in situ. For statistical analysis, the precursor conditions were divided into two major categories: low-grade intraurothelial neoplasia (mild to moderate dysplasia, LGIN) and high-grade intraurothelial neoplasia (severe dysplasia and carcinoma in situ, HGIN). The TCC were classified according to the three-tier histologic grading system of the World Health Organization. The growth pattern of papillary versus nonpapillary or solid tumors and the depth of invasion were also recorded. In four of five cystectomy specimens, a single focus of grade 3 nonpapillary TCC invading the muscularis propria was present and was accompanied by extensive precancerous lesions ranging from mild dysplasia to carcinoma in situ. In the remaining case, multiple foci of TCC were present. One focus represented a grade 3 nonpapillary TCC with transurothelial invasion of the bladder wall and involvement of perivesical adipose tissue. Two additional foci of carcinoma represented grade 3 papillary TCC without invasion. Like the other four cases, this case exhibited changes ranging from mild dysplasia to carcinoma in situ over extensive areas of the urinary bladder mucosa. The results of microscopic evaluation of individual mucosal samples were recorded and stored in a computer database as histologic maps.

Superimposed Histologic and Genetic Maps

The hypervariable markers were selected and used as previously described (Chaturvedi et al., 1997; Czerniak et al., 1999). In brief, a set of 38 hypervariable markers (Research Genetics, Huntsville, Ala., USA) mapped to chromosome 13, was selected using an updated Genethon microsatellite map (Dib et al., 1996). The allelic patterns of markers were resolved on polyacrylamide gel after their amplification using the polymerase chain reaction. A minimum of 50% reduction in signal intensity documented by densitometric measurements was required to be considered evidence of loss of heterozygosity (LOH). Testing was performed in two phases. Initially, all markers were analyzed on paired, nontumor versus invasive tumor DNA samples. The markers with evidence of LOH were subsequently used on all mucosal samples to generate superimposed histologic and genetic maps.

Analysis of Data

The data were organized and analyzed as previously described (Chaturvedi et al., 1997; Czerniak et al., 1999). In brief, the results of testing with hypervariable markers were entered into the data files and superimposed over the histologic maps. Initial raw data consisted of chromosomal vectors with a list of ordered markers, their alterations and coordinates for locations of mucosal samples, which could be used to plot their relation to microscopically classified urothelial changes. Superimposing plots of genetic changes over the histologic maps allowed analysis of which areas of bladder mucosa had altered markers and whether they had a relationship to intraurothelial precursor conditions and TCC.

Three-dimensional displays of allelic losses in relation to the progression of neoplasia from precursor intraurothelial conditions to invasive cancer in individual cystectomy specimens were generated and initially analyzed by the nearest-neighbor algorithm (Hartigan 1975). The relationship between altered markers and the progression of urothelial neoplasia from precursor conditions to invasive cancer revealed by superimposed histologic and genetic mapping were tested by a modified LOD score analysis (Ott 1991). Cumulated LOD scores were calculated at variable θ=0.01, 0.05, and 0.09. A pattern of LOD scores ≧3 at θ=0.01 or 0.09 and LOD scores <3 at θ=0.5 for the same marker was considered significant. A summary of raw data used for assembly of the genetic model is provided in Table 13.

TABLE 13
Raw Data Used For Assembly Of Genetic Model Of Human
Urinary Bladder Carcinogenesis

| Samples tested: | |
|---|---|
| NU | 53 |
| LGIN | 82 |
| HGIN | 50 |
| TCC | 49 |
| TOTAL | 234 |
| Chromosome markers | |
| tested | |
| CHROMOSOME 4 | 45 |
| Chromosome 8 | 43 |
| Chromosome 9 | 52 |
| Chromosome 11 | 47 |
| Chromosome 17 | 38 |
| TOTAL | 225 |
| First screening of paired | |
| normal and tumor DNA | |
| Chromosome 4 | 630 |
| Chromosome 8 | 602 |
| Chromosome 9 | 728 |
| Chromosome 11 | 658 |
| Chromosome 17 | 532 |
| TOTAL | 3150 |
| Secondary screening of | |
| all mucosal samples | |
| Chromosome 4 | 900 |
| Chromosome 8 | 565 |
| Chromosome 9 | 1012 |
| Chromosome 11 | 1163 |
| Chromosome 17 | 1152 |
| TOTAL | 4792 |
| Markers with statistically | |
| significant relation to | |
| urothelial neoplasia | |
| NU | 21 |
| LGIN | 28 |
| HGIN | 27 |
| TCC | 23 |
| *TOTAL | 47 |

*As the same marker may be altered significantly in different phases of neoplasia, the total number of markers is not the sum of the above 4 numbers. NU, normal urothelium; LGIN, low-grade intraurothelial neoplasia; HGIN, high-grade intraurothelial neoplasia; TCC, transitional cell carcinoma.

Results: Superimposed Histologic and Genetic Mapping

The initial testing of paired normal and tumor DNA samples from the same patient revealed loss of heterozygosity in 12 out of 38 tested markers on chromosome 13. No shortening or expansion of the repetitive sequences was identified and none of the cystectomy cases showed evidence of chromosone 13 monosomy. Testing of alterations on multiple samples from the same patient revealed the same pattern of allelic loss, i.e. the same allele was always altered, indicating the clonal relationship among the samples with altered markers.

The superimposition of microsatellite alterations over the histologic maps disclosed two basic distribution patterns of LOH: scattered (in the form of several isolated foci) and plaque-like. Some of the plaque-like alterations involved large areas of urinary bladder mucosa encompassing various precursor conditions, i.e. LGIN and HGIN, and even some adjacent areas of morphologically normal urothelium (FIG. 27). Such findings indicated that the alterations occurred early in the process of urothelial neoplasia and were associated with clonal expansion of abnormal urothelial cells involving large areas of urinary bladder mucosa. Alterations of some markers were restricted to specific stages of neoplasia, e.g. invasive carcinoma or invasive carcinoma with adjacent HGIN, indicating that the alterations were associated with the late phases of the process and possibly with invasive growth.

The three-dimensional patterns of allelic losses generated by the nearest neighbor analysis disclosed that markers which showed scattered foci of alterations were in fact located within the field change in which other chromosomal regions were deleted and involved larger areas of urinary bladder mucosa. An example of three-dimensional pattern of LOH in a single cysectomy specimen disclosed by the nearest neighbor analysis is shown in FIG. 1. The marker D13S154 located approximately 0.5cM from the RB gene developed LOH early in the process and was associated with clonal expansion of abnormal urothelial cells occupying large areas of bladder mucosa. LOH of the marker D13S171 within the BRCA2 gene in the 12q region and of the marker D13S154 mapped to the 13q31-32 region were later events confined to the areas of HGIN and invasive TCC.

These analyses performed on 234 DNA samples corresponding to precursor lesions and invasive TCC of five cystectomies identified the three minimal regions of allelic losses: 13q12 (D13S171), 13q14 (D13S291, RB1, D13S164, D13S268), 13q31 (D13S271). The significance of LOH for the development and progression of urothelial neoplasia in these regions was defined by the cumulative LOD scores calculated individually for LGIN, HGIN, and TCC as well as for the adjacent urothelium without microscopically recognizable preneoplastic conditions.

The 13q14 region contained a 4.8 cM minimal deleted segment flanked by D13S263 and D13S284 markers and centered around the RB1 gene. Allelic losses in this region represented early events in the development of urothelial neoplasia corresponding to LGIN and were associated with clonal expansion of abnormal urothelial cells involving large areas of bladder mucosa. Direct involvement of the RB1 gene with LOH of in VTRL region and the absence of immunohistochemically detectable RB protein was documented in two cystectomy cases. In two additional cases the markers located approximately 0.5 cM telomerically from the RB gene exhibited LOH in early phases of urothelial neoplasia. In these cases there was no evidence of direct involvement of the RB gene i.e. there was retention of heterozygosity of the RBI and VTRL marker with the normal heterogeneous pattern of RB protein expression documented immunohistochemically. This data confirmed the involvement of the RB1 gene in the early events of urothelial neoplasia and strongly suggests the presence of another putative tumor suppressor gene within the same locus.

The LOD score analysis revealed that an 4.8 cM segment mapped to 13q14, flanked by D13S263 and D13S284 markers and centered around the RB1 locus represented a critical deleted region involved in the early phases of urothelial carcinogenesis preceding the development of microscopically recognizable intraurothelial, preneoplastic changes. Allelic losses in this area were identified in 4 of 5 tested cystectomy specimens implicating its frequent involvement in urinary bladder neoplasia.

The LOD score analysis of allelic losses in the 13q12 region revealed a 3.2 cM deleted segment flanked by markers D13S260 and D13S268 centered around the marker D13S171. This allelic losses were associated with the development of high-grade intraurothelial neoplasia and progression to invasive disease and were identified in 3 of 5 tested cystectomy specimens.

The similar analysis of allelic losses in the 13q31 region showed another 4.0 cM segment flanked by markers D13S170 and D13S266 centered around the marker D13S271. The allelic losses in this area exhibited statistically significant LOD scores in association with the development of preneoplastic changes as well as high grade changes and invasive cancer, however they were identified only in one of five cystectomy specimens.

### Example 10

### Mapping and Genome Sequence Analysis of Chromosome 5 Regions Involved in Bladder Cancer Progression

Whole-Organ Histologic and Genetic Mapping

Radical cystectomy specimens from five patients with previously untreated sporadic high grade invasive transitional cell carcinoma (TCC) of the bladder were used for the whole-organ histologic and genetic mapping as previously described (Chaturvedi et al, 1997; Czerniak et al, 1999; and 2000). All patients were men and their ages ranged from 47 to 78 years (mean=66.4±11.9 years SD).

In brief, each fresh cystectomy specimen was opened longitudinally along the anterior wall of the bladder and pinned down to a paraffin block. The entire mucosa was than divided into 1×2 cm rectangular samples and evaluated microscopically on frozen sections. The tissue of interest was microdissected from the frozen block and used to prepare a urothelial cell suspension by mechanically scrapping the urothelial mucosa or gentle shaking invasive tumor samples. Only those specimens that yielded more than 90% of microscopically recognizable intact urothelial or tumor cells in each sample were accepted for the study and used for DNA extraction. This procedure provided 49, 39, 65, 42, and 39 DNA samples from each cystectomy specimen that corresponded to microscopically identified intraurothelial precursor conditions or invasive carcinoma. As a control, DNA extracted from the peripheral blood lymphocytes and/or from normal tissue in the resected specimen of each patient was used.

The intraurothelial precancerous changes were classified as mild, moderate, and severe dysplasia or carcinoma in situ. The tumors were classified according to the three-tier histologic grading system of the World Health Organization (Mostofi, 1999). The growth pattern of papillary versus nonpapillary or solid tumors and the depth of invasion were also recorded. In four of the five cystectomy specimens, a single focus of grade 3 nonpapillary urothelial carcinoma invaded the muscularis propria and was accompanied by extensive precancerous lesions ranging from mild dysplasia to carcinoma in situ. In the remaining case, multiple foci of carcinoma were present. One focus represented a grade 3 nonpapillary urothelial carcinoma with transmural invasion of the bladder wall and involvement of perivesical adipose tissue. Two additional foci of carcinoma represented grade 3 papillary urothelial carcinoma without invasion. Like the other four cases, this case exhibited changes ranging from mild dysplasia to carcinoma in situ over extensive areas of the urinary bladder mucosa. The results of microscopic evaluation of individual samples from five cystectomy specimens were recorded and stored in a computer database as histologic maps.

Microsatellites

A set of primers for 38 microsatellite loci on chromosome 5 based on integrated sex averaged microsatellite map from Genethon (version March 1966) and updated by Cooperative Human Linkage Center (version 4.0) was obtained from Research Genetics (Huntsville, Ala., USA). The markers selected for testing exhibited high levels of heterozygosity and uniform distribution covering all regions of chromosome 5. FIG. 8 lists hypervariable markers and their positions on chromosome 5. The allelic patterns of markers were resolved on 6% polyacrylamide gels after their amplification using polymerase chain reaction as previously described (Chaturvedi et al, 1997). A minimum 50% reduction in signal intensity was required to be considered as evidence of LOH. Tests with questionable results were repeated. In such cases, the densitometric measurements were performed to ensure objective reading of the data. Testing of markers was performed in two steps. Initially, all markers were tested on paired normal and tumor DNA samples. This revealed LOH in 12 markers, which were tested on all mucosal DNA samples by whole-organ histologic and genetic mapping.

Analysis of LOH Data

The data were organized and analyzed as previously described (Chaturvedi et al, 1997; Czerniak et al, 1999; and 2000). In brief, the information on LOH in individual loci was entered into the data files and superimposed over the histologic maps. Initial data consisted of chromosomal vectors with a list of LOH in individual loci and coordinates for locations of mucosal samples, which could be used to plot the distribution of LOH to microscopically classified urothelial changes. By superimposing plots of LOH over the histologic maps, we identified the areas of bladder mucosa with altered markers and analyzed their relationship to intraurothelial precursor conditions and invasive cancer. Three-dimensional displays of LOH in relation to the progression of neoplasia from precursor intraurothelial conditions to invasive cancer were generated and initially analyzed by the nearest-neighbor algorithm (Hartigan, 1975).

The relationship between altered markers and the progression of urothelial neoplasia from precursor conditions to invasive cancer was tested by a binomial maximum likelihood analysis, and the significance of the relationship was expressed as LOD score (Ott, 1991). We chose LOD scores because they represent a powerful method of likelihood analysis that can verify the statistical significance of the relationship among patterns of sequential events. The LOD scores were applied in their generic mathematical sense as likelihood tests of events, not as in their common use to test the linkage in familial disorders with meiotic segregation of the phenotype at a recombination fraction Θ=0.5. In sporadic cancer when microscopically defined stages of cancer progression are used as standards of sequential events and there is a mitotic transmission of the phenotype, the null hypothesis is more appropriately verified at a recombination factor Θ differing from 0.5. Hence, cumulated LOD scores were calculated at variable Θ=0.01, 0.5, and 0.99. A pattern of LOD scores ≧3 at Θ=0.01 or Θ=0.99 and LOD scores <3 at Θ=0.5 for the same marker was considered significant. The strongest association between altered marker and neoplasia was when a LOD score was ≧3 and Θ=0.99 and 0.5 and <3 at Θ=0.01. Stringency 1 designated LOD scores for specific stages of neoplasia. Stringency 2 designated LOD scores for progression to higher stages of neoplasia. The analysis of relationship among LOH in individual loci and various clinico-pathological parameters of tumors and of voided urine samples was tested by Gehan's generalized Wilcoxon, and log-rank tests (p≦0.05 was considered significant).

Frequency of Allelic Losses on Chromosome 5 in Bladder Tumors and Voided Urine Samples

The markers of chromosome 5 that were identified as significantly altered by the whole histologic and genetic mapping were tested in 37 tumor and 29 voided urine samples. The tumors were classified according to the three-tier histologic grading system of the World Health Organization (Mostofi, 1999). The growth pattern, tumor grade and depth of invasion were also recorded. Levels of invasion were recorded according to the TNM staging system (Sobin et al, 1997). DNA was extracted from individual bladder tumors and sediments of voided urine samples as previously described (Chaturvedi et al, 1997). For controls, DNA was also extracted from the peripheral blood lymphocytes and/or normal tissue in the resected specimens from each patient.

Analysis of Contigs and Genome Sequence Databases Spanning the Deleted Regions

The initial resource available for the whole-organ histologic and genetic mapping of deleted regions on chromosome 5 consisted of a list of hypervariable markers based on integrated sex averaged micosatellites maps from Genethon and Cooperative Human Linkage Center. However, human genome sequence-based databases with more accurate physical maps become available during our studies. Thus, to relate our data to sequence maps of human genome, we initially looked for overlap between the original sets of markers which defined the deleted regions and those used to generate the current version of GeneMap'99 (http://www.ncbi.nlm.nih.gov/genemap99/). This resource represents the most complete melding of the microsatellite-based genetic map data from Genethon (http://www.genethon.fr/) with the GeneBridge 4 (GB4) and Stanford G3 radiation hybrid panel-based physical map produced by the International Radiation Hybrid Mapping Consortium (http://www.ncbi.nlm.nih.gov/genemap99/page. cgi?F=Consortium. html).

While some of the original Marshfield sex-averaged markers defining the deleted regions can be found in GeneMap'99, substitutes for those not found were proposed based primarily on proximity of physical distances. The resources used for these substitutions included the “Golden Path” Genome Browser (http://genome.ucsc.edu/), containing the whole-genome fingerprint map from Washington University (http://genome.wustl.edu/gsc/human/human_database.shtml), the sequence-based mapping tools at the Ensembl website produced at the European Bioinformatics Institute (http://www.ensembl.org/), and the integrated MapViewer browser from the NCBI (http://www.ncbi.nlm.nih.gov/cgi-bin/Entrez/hum_srch?chr=hum_chr.inf&query). These same resources, together with NCBI's LocusLink (http://www.ncbi.nlm.nih.gov/LocusLink/), were used to scan the marker-defined deleted regions for both known genes and EST clusters based on Unigene (http://www.ncbi.nlm.nih.gov/UniGene/Hs.Home.html). The Baylor College of Medicine Search Launcher (http://www.hgsc.bcm.tmc.edu/SearchLauncher/) provided the portal and integration for these links.

After reorientation of contigs and sequence databases, multiple electronic PCR searches were performed to find and relocate the original set of markers on the GB4 and sequence maps. As a general rule we attempted to locate the original markers and substitute GB4 markers within a single BAC clone. Since in the majority of instances complete continuous sequences of BAC clones were not available yet, it was impossible to find the exact order of paired original and substitute markers within the target BAC clone. When the original and substitute markers were not located within the same BAC clone the most proximal substitute markers within the contigs spanning the analyzed regions were provided.

RESULTS: Whole-Organ Histologic and Genetic Mapping

The initial testing of paired normal and tumor DNA samples from the same patient identified loss of heterozygosity (LOH) in 12 of 38 hypervariable markers. No expansion or shortening of repetitive sequences was identified. None of the cystectomy specimens showed evidence of continuous allelic losses involving large portions of chromosome 5 or complete loss of the entire chromosome, precluding precise mapping of smaller regions. The list of tested markers, their alterations, and chromosomal locations is provided in FIG. 8.

Testing of markers with LOH on multiple mucosal samples of the same cystectomy specimen always revealed a loss of the same allele, implicating a clonal relationship among cells from individual mucosal samples (FIG. 43). By superimposing distributions of LOH in individual loci over the histologic maps, we identified two basic distribution patterns of LOH involving urinary bladder mucosa, scattered and plaque-like (FIG. 43). Some of the plaque-like alterations involved large areas of urinary bladder mucosa with various precursor conditions and even some adjacent areas of morphologically normal urothelium. Such patterns of mucosal involvement implied that LOH occurred early in the development of urothelial neoplasia, even before microscopically recognizable preneoplastic conditions developed. However, smaller plaques of LOH restricted to areas of severe dysplasia/carcinoma in situ or invasive cancer represented late hits associated with progression to the invasive phenotype.

Three-dimensional patterns of LOH in individual chromosome 5 loci in relation to progression of neoplasia from precursor conditions to invasive cancer were generated by nearest neighbor analysis. None of the mucosal areas with LOH was rejected by the nearest neighbor algorithm, indicating that scattered foci of alterations were in fact located within the larger field change in which other regions of chromosome 5 showed LOH.

For a binomial maximum likelihood analysis the intraurothelial precancerous conditions were classified into two groups: low-grade intraurothelial neoplasia (mild to moderate dysplasia, LGIN) and high-grade intraurothelial neoplasia (severe dysplasia and carcinoma in situ, HGIN). Analysis of LOD scores showed that the markers exhibiting LOH with a statistically significant relationship to the development and progression of urothelial neoplasia were clustered in a large approximately 70 cM 5q13.3-q32 region containing several smaller discontinuous areas of allelic losses involving 5q13.3-q22, 5q22-q31. 1, and 5q31.1-q32. The deleted regions defined by their flanking markers and their predicted size as well as the list of markers within these regions with LOH are provided in FIG. 8. The allelic losses within the region 5q13.3-q22 showed LOH of a marker D5S421 associated with the development of LGIN which also could be identified in the adjacent areas of microscopically normal urothelium, implicating its involvement in early phases of urothelial neoplasia antecedent to the development of microscopically recognizable preneoplastic conditions. The remaining markers (D5S428, D5S346, and a marker located within the APC gene) mapping to the same region showed LOH in later phases of urothelial neoplasia associated with the development of HGIN progressing to invasive bladder cancer. The adjacent minimally deleted region within the 5q22-q31.1 involved four markers: MCC, D5S659, D5S2055, and D5S818. The marker D5S659 showed allelic losses associated with the development of LGIN. The three remaining markers mapping to this region developed LOH in the late phases of urothelial neoplasia i.e. HGIN progressing to invasive carcinoma. Additional smaller region of deletions was found in 5q31.1-q32 and involved markers located within the IRF1 and CSF1R genes. The allelic losses within the CSF1R and IRF1 genes were identified in association with development of LGIN. A separate deleted region mapping to 5q34 involved marker D5S1465, which revealed LOH in association with the development of HGIN progressing to invasive carcinoma.

Frequency of Allelic Losses on Chromosome 5 in Bladder Tumors and Voided Urine Samples

Markers showing LOH with statistically significant relationship to progression of urinary neoplasia which clustered in 4 distinct chromosomal regions including their nearest non-altered flanking markers were tested on 37 tumors and 29 voided urine samples of patients with bladder cancer and paired nontumor DNA from peripheral blood lymphocytes (Table 14). LOH of at least one marker could be identified in 38.4% of informative tumors and 58.6% of informative voided urine samples. The highest frequency of LOH in both tumor and voided urine samples was found in region mapping to 5q22-q31.1 and could be identified in 27.0% and 27.5% of cases, respectively. Second most frequently deleted region mapping to 5q13.3-q22 showed LOH in approximately 24% of tumor and voided urine samples. In two remaining loci mapping to 5q31.1-q32 and 5q34 the allelic losses in both tumor and voided urine samples could be identified in 18% or less of the cases. The statistical analysis of frequency of LOH in individual loci and minimally deleted regions on chromosome 5 have shown that none of the LOH could be related to specific pathogenetic subsets histologic grade or stage of the tumor. Although, the allelic losses within 5q13.3-q22 were the most frequent the markers with LOH mapping to this area did not form a distinct narrow region of allelic losses. On the other hand, the two neighbor markers, D5S2055 and D5S818, mapping to 5q22-q31.1 defined a distinct region of allelic losses that could be identified in 21.6% and 27.5% of bladder tumor and urine samples, respectively. Thus, the minimally deleted region flanked by markers D5S659 and D5S808, spanning approximately 9 cM, may contain tumor suppressor genes with important roles in urinary bladder carcinogenesis.

TABLE 14
Frequency of allelic losses at different regions on chromosome 5 in
bladder TCC and voided urine samples.

| | | Frequency of LOH (%) | | | |
|---|---|---|---|---|---|
| | | Bladder Tumor | | Voided Urine Samples | |
| Deleted | | Sample (n = 37) | | (n = 29) | |
| regions | Markers | | Region | Marker | Region |
| 5q13.3-q22 | D5S428 | 19.4 | 24.3 | 13.6 | 24.1 |
| | D5S421 | 2.8 | | 12.0 | |
| | APC | 5.9 | | 7.7 | |
| | D5S346 | 19.4 | | 7.7 | |
| 5q22-q31.1 | MCC | 5.6 | 27.0 | 0.0 | 27.5 |
| | D5S659 | 9.1 | | 0.0 | |
| | D5S2055 D5S818 | 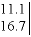 | 21.6 | 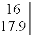 | 27.5 |
| 5q31.1-q32 | IRF1 | 8.3 | 8.1 | 12.0 | 17.2 |
| | CSF1R | 2.7 | | 11.5 | |
| 5q34 | D5S1465 | 12.5 | 12.5 | 4.0 | 4.0 |

The distinct region of allelic losses mapping to 5q22-q31.1 defined by the two neighbor markers (D5S2055 and D5S818) is identified by a solid vertical bar followed by a combined % of LOH for these markers. Raw data used for this analysis can be obtained from (http://www.mdanderson.org/Departments/GenomeMaps/)

Analysis of Contigs and Genome Sequence Databases Spanning the Deleted Regions

The analysis of human genome contig and sequencing databases spanning the deleted regions on chromosome 5 is summarized in FIG. 32. The 4 deleted regions on chromosome 5 contain 138 known genes. In addition, multiple EST were assigned to individual deleted regions identifying several smaller gene-rich areas. The most frequently deleted region mapping to 5q22-q31.1 contained areas with high densities of EST and known genes, some of them with putative tumor suppressor activities, further supporting a concept of its potential pathogenetic relevance for bladder carcinogenesis.

### Example 11

### Genetic Mapping and DNA Sequence-Based Analysis of Deleted Regions on Chromosome 16 Involved in Progression of Bladder Cancer from Occult Preneoplastic Conditions to Invasive Disease

Histologic and Genetic Mapping

Five cystectomy specimens with invasive urothelial carcinoma were used for whole-organ histologic and genetic mapping and were prepared as previously described (Chaturvedi et al., 1997). All cases represented previously untreated sporadic carcinoma of the bladder. None of the cases occurred in the known familial syndrome predisposing to the development of urinary bladder cancer. All patients were males, and their age ranged from 47 to 78 years (mean=66.4±11.9 years SD). The tissue of interest was identified microscopically and microdissected from the frozen block. DNA was extracted from cell suspensions containing at least 90% microscopically recognizable intact urothelial cells. Cystectomy specimens yielding less pure cell suspensions were not included in this study.

We obtained 49, 39, 65, 42, and 39 mucosal samples respectively from each bladder. In four cases, a single focus of grade 3, nonpapillary urothelial carcinoma invading the muscularis propria, was present. It was accompanied by extensive precancerous lesions ranging from mild dysplasia to carcinoma in situ. In one case (map 3), multiple foci of carcinoma were present. One focus represented a grade 3 nonpapillary urothelial carcinoma with transmural invasion of the bladder wall and involvement of the perivesical adipose tissue. Two additional foci of carcinoma represented grade 3 papillary urothelial carcinoma without invasion. Like the other four cases, extensive areas of the urinary bladder mucosa in this case exhibited changes ranging from mild dysplasia to carcinoma in situ.

Tumor, Voided Urine Samples, and Clinico-Pathological Data

Fresh samples of urinary bladder tumors from 28 patients and voided urine samples from 25 patients with TCC were used to study the allelic losses. The markers of chromosome 16 that were identified as significantly altered by the superimposed histologic and genetic mapping were tested in 28 tumor samples and 25 voided urine samples. The intraurothelial precancerous changes were microscopically classified as mild, moderate, or severe dysplasia or as carcinoma in situ. The TCCs were classified according to the three-tier histologic grading system of the World Health Organization (Mostofi et al., 1999). The growth pattern(papillary versus nonpapillary), and depth of invasion according to the TNM staging system were also recorded (Sobin and Wittekind, 1997). DNA was extracted from individual bladder tumors and sediments of voided urine samples as previously described (Chaturvedi et al., 1997). For controls, DNA was also extracted from the peripheral blood lymphocytes and/or normal tissue in the resected specimens from each patient.

Microsatellites

A set of primers for 30 microsatellite markers on chromosome 16 based on an updated Genethon microsatellite map was purchased from Research Genetics (Huntsville, Ala., USA), (Gyapay et al. 1994). The markers selected for testing exhibited high levels of heterozygosity and relatively uniform distribution, i.e. they covered all regions of chromosome 16. The allelic patterns of markers were resolved on polyacrylamide gels after their amplification using the polymerase chain reaction as previously described (Chaturvedi et al., 1997). A minimum 50% reduction in signal intensity was required to be considered evidence of LOH. Tests with questionable results were repeated. In such cases the densitometric measurements were performed to ensure objective reading of the data. Testing of markers was performed in two phases. Initially, all 30 markers were tested on paired non-tumor versus tumor DNA samples. This revealed LOH of 13 markers, which were subsequently tested on all mucosal samples to generate whole-organ histologic and genetic maps.

Analysis of LOH Data

The data were analyzed as previously described (Chaturvedi et al., 1997). In brief, three-dimensional displays of LOH distribution patterns in relation to progression of the neoplasia from precursor intraurothelial conditions to invasive cancer were generated and initially analyzed by the nearest-neighbor analysis (Hartigan, 1975). The significance of LOH in individual markers for progression of urothelial neoplasia from precursor conditions to invasive carcinoma was tested by a binomial maximum likelihood analysis, and the significance of the relationship was expressed as a LOD score. Cumulative LOD scores were calculated at variable 0 (0.01, 0.5, and 0.99). Stringency level 1 designated LOD scores for specific stages of neoplasia. Stringency level 2 designated LOD scores for progression to higher stages of neoplasia. The pattern of LOD score 3 at=0.01 or 0.99 and LOD score <3 at=0.5 for the same marker were considered significant. The strongest association between an altered marker and neoplasia was when a LOD score was 3 at=0.99 and 0.5 and <3 at=0.01. In this approach, the geographic relationship between LOH and specific phases of urothelial neoplasia was more important than the absolute number of alterations in individual mucosal samples and/or cystectomy specimens. Therefore, LOH of a tested marker seen in several cystectomy specimens but without a geographic relationship to specific phases of neoplasia was not identified as statistically significant. On the other hand, LOH of limited number of samples which corresponded to distinct phases of bladder cancer development and progression was typically identified as significant. The use of LOD scores in this analysis was not the same as that commonly used in linkage analysis of familial genetic predisposition for diseases (Ott, 1991). Rather, it was intended to be used in its generic mathematical sense as a likelihood test of events (Brownlee, 1965). We used the LOD score variant of the likelihood test, as many researchers are more familiar with approximate levels of significance when expressed in this form. The relationships among LOH in individual loci and various clinico-pathological parameters of tumors and of voided urine samples were tested by Gehan's generalized Wilcoxon and log-rank tests (p≦0.05 was considered significant).

Analysis of Contig and Sequence Data

The initial plan for our whole-organ histologic and genetic mapping of chromosome 16 involvement in bladder neoplasia was based on a map of hypervariable markers from Genethon, version March, 1996. However, during the course of this study rapidly emerging human genome sequence data with more accurate physical and sequence-based maps became available. To relate our findings to these new resources, the markers defining deleted regions of chromosome 16 were reoriented with the set of markers used to generate the current version of GeneMap99(htp://www.ncbi.nlm.nih.gov/genemap99/). GeneMap99 represents the most complete melding of the microsatellite-based genetic map data from Genethon (http://www.genethon.fr/) with the GB4 and G3 radiation hybrid panel-based physical map produced by the International Radiation Hybrid Mapping Consortium (http://www.ncbi.nlm.nih.gov/genemap99/page. cgi?F=Consortium. html). While some of the Marshfield sex-averaged markers used in this analysis can be found in GeneMap99, substitutes for those not found were proposed based primarily on the proximity of physical distances and in most instances location within the same BAC clone. The resources used to find substitute markers included the “Golden Path” Genome Browser (http://genome.ucsc.edu/), based on the whole-genome fingerprint map assembly from Washington University (http://genome.wustl.edu/gsc/human/human_database.shtml), the sequence-based mapping tools at the Ensembl website produced at the European Bioinformatics Institute (http://www.ensembl.org/), and the highly integrated MapViewer browser from the NCBI (http://www.ncbi.nlm.nih.gov/cgi-bin/Entrez/hum_srch?chr=hum_chr.inf&query). Finally, these resources, together with NCBI's LocusLink (http://www.ncbi.nlm.nih.gov/LocusLink/) were used to scan the deleted regions for both known genes and EST clusters based on Unigene (http://www.ncbi.nlm.nih. gov/UniGene/Hs.Home.html), while the BCM Search Launcher (http://www.hgsc.bcm. tmc.edu/SearchLauncher/) provided the portal and integration for these links. After reorientation of contigs based on multiple substitute markers, electronic PCR searches were performed to relocate the original set of markers defining the deleted regions. Since in most instances the continuous sequence of individual BAC clone was not available, the exact order of the original Genethon versus neighbor substitute markers within the single BAC clone is not known.

Results: Whole-Organ Histologic and Genetic Mapping

The initial testing of paired normal and invasive tumor DNA samples from the same patient revealed loss of heterozygosity (LOH) in 11 of 30 tested markers mapped to chromosome 16 (FIG. 19). No shortening or expansion of the repetitive sequences was identified. None of the cystectomy cases used for whole-organ histologic and genetic mapping showed evidence of chromosome 16 monosomy, i.e. none of the cases showed LOH of all informative markers, which would indicate complete loss of chromosome 16. Testing of alterations on multiple samples from the same patient revealed the same pattern of allelic loss, i.e., the same allele was always lost, indicating a clonal relationship exists among the samples with an altered marker (FIG. 44A). The superimposition of distributions of allelic losses in individual markers over the histologic maps disclosed two basic patterns of chromosome 16 deletions: scattered and plaque-like. Some of the allelic losses involved large areas of urinary bladder mucosa encompassing various precursor conditions and even some adjacent areas of morphologically normal urothelium, which implicated their involvement in early phases of urothelial neoplasia (FIG. 44B). On the other hand, some markers exhibited LOH restricted to severe dysplasia/carcinoma in situ and invasive carcinoma only, suggesting their involvement in the later phases of urothelial neoplasia progressing to invasive disease. The patterns of LOH distribution of the entire chromosome in individual cystectomies were generated by the nearest neighbor analysis (FIG. 44C). The nearest neighbor analysis disclosed that scattered foci of alterations with no apparent relationship to specific phases of neoplasia were in fact located within the field change in which other chromosomal regions were deleted and involved larger areas of the urinary bladder mucosa.

For the purpose of binomial maximum likelihood analysis the intraurothelial precancerous changes were classified into two major groups: low-grade intraurothelial neoplasia (mild and moderate dysplasia; LGIN) and high-grade intraurothelial neoplasia (severe dysplasia and carcinoma in situ; HGIN). The analysis of LOD scores revealed that the markers with a statistically significant relationship to the development and progression of urothelial neoplasia were located in several distinct chromosome 16 regions: p13.3 (D16S513); p13.1 (D16S500); q12.1 (D16S541, D16S415); q22.1 (D16S592); q24 (D16S505, D16S520). The location of these regions, their predicted size, and the position of the nearest flanking markers are shown in FIG. 19. The regions mapping to p13.1, q22.1, and q24 developed allelic losses early during the development of urothelial neoplasia, involving areas of urinary bladder mucosa with LGIN as well as adjacent areas of normal urothelium. In contrast, a p13.3 developed LOH in late phases of urothelial neoplasia, and it was associated with HGIN progressing to invasive carcinoma. In addition, allelic losses within the q12.1 were statistically significant for the development of early phases of urothelial neoplasia such as LGIN, but they were not associated with progression to HGIN and invasive carcinoma. Such patterns of alteration suggested that LOH in this area may not be functionally significant for the progression of preneoplastic changes to invasive disease.

Testing of Allelic Losses on Chromosome 16 in Bladder Tumors and Voided Urine Samples

The markers that exhibited statistically significant relationships to the development and progression of urothelial neoplasia as revealed by the whole-organ histologic and genetic mapping as well as their nearest nonaltered flanking markers were tested on multiple bladder tumors and voided urine samples of the patient with bladder cancer corresponding to different pathogenetic subsets, grades, and stages of the disease (Table 15). The frequencies of alterations in individual markers as well as in their corresponding chromosomal regions are provided in Table 16. Alterations of at least one of the tested markers could be identified in 82.1% of tumors and 60.0% of voided urine samples of patients with TCC. Moreover alterations of multiple markers mapped to selected regions of chromosome 16 (>2 markers) could be identified in 39.3% of bladder tumor and 32.0% of voided urine samples of patients with bladder cancer. The allelic losses involving q12.1, p13.1, and q24 were the most frequent and could be identified in 46.4%, 28.6% and 21.4% of tumor samples, respectively. The alterations in these regions could be also documented in 20-32% of voided urine samples. Interestingly, the allelic losses of a single marker, D16S541, flanked by D16S409 and D16S415 and spanning 10 cM, could be identified in 28.6% of tumor and 20.0% of voided urine samples of the patient with bladder cancer defining the most frequently deleted region of chromosome 16 involved in urinary bladder cancer.

TABLE 15
Allelie losses of chromosome 16 identified in 28 tumor samples and 25 voided urine samples of patients with urinary bladder cancer

| Current status | | | | FU | Primary tumor | | | p13.3 | | | p13.1 | | | q12.1 | | | |
|---|---|---|---|---|---|---|---|---|---|---|---|---|---|---|---|---|---|
| No | Growth | Grade | Stage | Mo | Growth | Grade | Stage | D16S418 | D16S513 | D16S406 | D16S748 | D16S500 | D16S287 | D16S409 | D16S541 | D16S415 | D16S514 |

Tumor samples

| | | | | | | | | | | | | | | | | |
|---|---|---|---|---|---|---|---|---|---|---|---|---|---|---|---|---|
| 1 | 1 | Ta | 10 | P | 2 | Ta | X | 0 | 0 | 0 | 0 | 0 | [symbol] | 0 | 0 | 0 |
| 2 | | T4 | | P | 2 | T3 | 0 | 0 | 0 | 0 | 0 | 0 | [symbol] | 0 | φ | 0 |
| 3 | | | 48 | P | 2 | Ta | 0 | 0 | 0 | 0 | 0 | 0 | 0 | [symbol] | 0 | 0 |
| 4 | | | 46 | P | 2 | Ta | φ | 0 | 0 | 0 | 0 | 0 | 0 | 0 | 0 | 0 |
| 5 | | | 23 | P | 2-3 | Ta | 0 | φ | 0 | 0 | 0 | 0 | 0 | [symbol] | 0 | 0 |
| 6 | | T3 | 23 | P | 3 | T3 | 0 | 0 | 0 | φ | φ | 0 | 0 | [symbol] | 0 | 0 |
| 7 | | | | P | 3 | Ta | 0 | 0 | 0 | 0 | [symbol] | 0 | [symbol] | [symbol] | 0 | 0 |
| 8 | | T0 | 4 | P | 3 | T1 | [symbol] | [symbol] | 0 | 0 | 0 | 0 | 0 | 0 | 0 | φ |
| 9 | | | 25 | P | 3 | T1 | φ | φ | [symbol] | φ | 0 | 0 | 0 | 0 | 0 | 0 |
| 10 | | | 8 | P | 3 | T2 | 0 | 0 | 0 | 0 | 0 | X | 0 | 0 | 0 | 0 |
| 11 | | T3 | 16 | NP | 3 | T3 | [symbol] | 0 | 0 | 0 | 0 | 0 | 0 | 0 | [symbol] | 0 |
| 12 | | T3 | 12 | NP | 3 | T3 | 0 | 0 | 0 | 0 | 0 | 0 | 0 | 0 | [symbol] | 0 |
| 13 | 3 | T3 | 10 | NP | 3 | T3 | 0 | 0 | 0 | 0 | [symbol] | 0 | 0 | 0 | [symbol] | 0 |
| 14 | 3 | T3 | 6 | NP | 3 | T3 | φ | 0 | 0 | φ | φ | 0 | [symbol] | [symbol] | 0 | 0 |
| 15 | 3 | T3 | 6 | NP | 3 | T3 | 0 | 0 | 0 | 0 | 0 | 0 | 0 | [symbol] | 0 | 0 |
| 16 | | T2 | 19 | NP | 3 | T2 | 0 | 0 | 0 | X | 0 | 0 | 0 | [symbol] | 0 | 0 |
| 17 | | T2 | 93 | NP | 3 | T3 | 0 | 0 | 0 | 0 | 0 | 0 | 0 | [symbol] | 0 | 0 |
| 18 | 3 | T2 | 13 | NP | 3 | T2 | X | X | 0 | X | 0 | 0 | 0 | 0 | 0 | 0 |
| 19 | 3 | T2 | 15 | NP | 3 | T2 | X | X | 0 | X | 0 | 0 | 0 | 0 | 0 | 0 |
| 20 | 3 | T4 | 7 | NP | 3 | T4 | E | 0 | 0 | 0 | 0 | 0 | 0 | 0 | 0 | 0 |
| 21 | 3 | T2 | 23 | NP | 3 | T2 | 0 | X | 0 | X | 0 | 0 | X | 0 | 0 | 0 |
| 22 | 3 | T3 | 19 | NP | 3 | T3 | 0 | 0 | 0 | φ | φ | 0 | 0 | 0 | 0 | 0 |
| 23 | 3 | T3 | 45 | NP | 3 | T3 | 0 | [symbol] | 0 | [symbol] | 0 | 0 | 0 | 0 | 0 | 0 |
| 24 | 3 | T3 | 11 | NP | 3 | T3 | 0 | [symbol] | 0 | 0 | [symbol] | 0 | 0 | 0 | 0 | 0 |
| 25 | 3 | T3 | 11 | NP | 3 | T3 | 0 | 0 | 0 | 0 | [symbol] | 0 | 0 | 0 | 0 | 0 |
| 26 | 3 | T3 | 18 | NP | 3 | T3 | 0 | 0 | 0 | 0 | 0 | 0 | 0 | 0 | 0 | φ |
| 27 | 3 | T3 | 16 | NP | 3 | T3 | X | 0 | 0 | 0 | 0 | 0 | 0 | 0 | 0 | 0 |
| 28 | 3 | T3 | 22 | NP | 3 | T3 | 0 | φ | 0 | 0 | 0 | 0 | 0 | 0 | 0 | 0 |

Voided urine samples*

| | | | | | | | | | | | | | | | | | |
|---|---|---|---|---|---|---|---|---|---|---|---|---|---|---|---|---|---|
| 1 | | 2 | Ta | 55 | P | 1 | Ta | 0 | 0 | S | 0 | 0 | 0 | φ | S | 0 | 0 |
| 2 | | | To | 60 | P | 2 | Ta | 0 | 0 | [symbol] | 0 | 0 | 0 | [symbol] | [symbol] | 0 | 0 |
| 3 | | 2 | Ta | 140 | P | 2 | Ta | φ | φ | 0 | X | 0 | 0 | 0 | [symbol] | 0 | φ |
| 4 | P | 2 | Ta | 149 | P | 2 | Ta | 0 | [symbol] | 0 | X | X | [symbol] | 0 | 0 | 0 | 0 |
| 5 | P | 2 | Ta | 0 | P | 2 | Ta | 0 | 0 | 0 | 0 | 0 | 0 | 0 | X | X | 0 |
| 6 | | | To | 15 | P | 2 | Ta | 0 | 0 | 0 | 0 | 0 | 0 | 0 | 0 | 0 | 0 |
| 7 | | | To | 6 | P | 2 | T2 | 0 | φ | 0 | X | φ | φ | φ | 0 | φ | X |
| 8 | P | 2 | Ta | 8.5 | P | 2 | T1 | 0 | 0 | 0 | 0 | 0 | 0 | 0 | 0 | 0 | 0 |
| 9 | NP | 3 | T3a | 25 | NP | 3 | T1 | 0 | φ | 0 | 0 | 0 | φ | φ | 0 | [symbol] | φ |
| 10 | | 3 | Tis | 0.6 | NP | 3 | T2 | 0 | 0 | 0 | 0 | [symbol] | 0 | 0 | 0 | 0 | [symbol] |
| 11 | NP | 3 | T2 | 2.5 | NP | 3 | T2 | 0 | 0 | 0 | 0 | [symbol] | 0 | 0 | [symbol] | 0 | 0 |
| 12 | NP | 3 | T2 | 0 | NP | 3 | T2 | φ | 0 | 0 | φ | 0 | [symbol] | 0 | [symbol] | 0 | φ |
| 13 | | | To | 1 | NP | 3 | T2 | 0 | 0 | 0 | 0 | φ | 0 | [symbol] | 0 | 0 | 0 |
| 14 | NP | 3 | T3b | 1 | NP | 3 | T1 | 0 | 0 | 0 | 0 | 0 | 0 | 0 | 0 | 0 | 0 |
| 15 | | 3 | Tis | 1 | NP | 3 | T2 | 0 | 0 | 0 | [symbol] | 0 | 0 | 0 | 0 | 0 | 0 |
| 16 | NP | 3 | T4 | 4 | NP | 3 | T2 | 0 | 0 | 0 | 0 | 0 | 0 | 0 | 0 | 0 | 0 |
| 17 | | | To | 3 | NP | 3 | T2 | 0 | 0 | 0 | 0 | 0 | 0 | φ | X | 0 | φ |
| 18 | | 3 | Ta | 120 | NP | 3 | T3 | 0 | 0 | 0 | 0 | 0 | 0 | 0 | 0 | 0 | 0 |
| 19 | | | To | 0.6 | NP | 3 | T2 | 0 | 0 | 0 | 0 | 0 | 0 | 0 | 0 | 0 | 0 |
| 20 | NP | 3 | T1 | 1.6 | NP | 3 | T1 | 0 | 0 | 0 | 0 | 0 | 0 | 0 | 0 | 0 | 0 |
| 21 | NP | 3 | T3a | 1 | NP | 3 | T2 | 0 | 0 | 0 | 0 | 0 | 0 | 0 | 0 | 0 | 0 |
| 22 | | 3 | Tis | 0.6 | NP | 3 | T1 | 0 | 0 | 0 | 0 | 0 | 0 | 0 | 0 | 0 | 0 |
| 23 | P | 2 | T1 | | | | | 0 | [symbol] | 0 | 0 | 0 | 0 | 0 | 0 | 0 | 0 |
| 24 | | | To | 2 | | Tis | Tis | 0 | 0 | 0 | 0 | 0 | 0 | ? | [symbol] | 0 | [symbol] |
| 25 | NP | 3 | T3 | | | | | [symbol] | 0 | 0 | 0 | 0 | 0 | 0 | 0 | 0 | 0 |

| Current status | | | | FU | Primary tumor | | | q22.1 | | | q24 | | | q24 | | |
|---|---|---|---|---|---|---|---|---|---|---|---|---|---|---|---|---|
| No | Growth | Grade | Stage | Mo | Growth | Grade | Stage | D16S496 | D16S512 | D16S515 | D16S307 | D16S505 | D16S511 | D16S402 | D16S520 | D16S413 |
| Tumor samples | | | | | | | | | | | | | | | | |
| 1 | | 1 | Ta | 10 | P | 2 | Ta | 0 | 0 | 0 | [symbol] | 0 | 0 | 0 | X | 0 |
| 2 | | | T4 | | P | 2 | T3 | 0 | 0 | 0 | 0 | φ | 0 | 0 | 0 | 0 |
| 3 | | | | 48 | P | 2 | Ta | 0 | 0 | 0 | 0 | 0 | φ | 0 | 0 | 0 |
| 4 | | | | 46 | P | 2 | Ta | 0 | 0 | 0 | 0 | 0 | 0 | 0 | 0 | 0 |
| 5 | | | | 23 | P | 2-3 | Ta | 0 | 0 | 0 | 0 | φ | 0 | φ | φ | 0 |
| 6 | | | T3 | 23 | P | 3 | T3 | 0 | 0 | 0 | 0 | 0 | 0 | 0 | 0 | 0 |
| 7 | | | | | P | 3 | Ta | [symbol] | 0 | [symbol] | ρ | 0 | 0 | 0 | 0 | 0 |
| 8 | | | T0 | 4 | P | 3 | T1 | 0 | 0 | 0 | 0 | φ | φ | φ | 0 | 0 |
| 9 | | | | 25 | P | 3 | T1 | 0 | [symbol] | 0 | X | 0 | 0 | φ | 0 | 0 |
| 10 | | | | 8 | P | 3 | T2 | 0 | 0 | [symbol] | 0 | 0 | 0 | 0 | 0 | 0 |
| 11 | | | T3 | 16 | NP | 3 | T3 | 0 | 0 | 0 | 0 | 0 | 0 | 0 | 0 | 0 |
| 12 | | | T3 | 12 | NP | 3 | T3 | 0 | 0 | 0 | 0 | 0 | 0 | 0 | 0 | 0 |
| 13 | | 3 | T3 | 10 | NP | 3 | T3 | 0 | 0 | 0 | 0 | 0 | 0 | 0 | [symbol] | 0 |
| 14 | | 3 | T3 | 6 | NP | 3 | T3 | 0 | 0 | [symbol] | 0 | 0 | φ | 0 | 0 | [symbol] |
| 15 | | 3 | T3 | 6 | NP | 3 | T3 | 0 | 0 | 0 | [symbol] | 0 | 0 | 0 | [symbol] | 0 |
| 16 | | | T2 | 19 | NP | 3 | T2 | 0 | 0 | 0 | 0 | 0 | φ | 0 | 0 | 0 |
| 17 | | | T2 | 93 | NP | 3 | T3 | 0 | 0 | 0 | [symbol] | 0 | 0 | 0 | 0 | 0 |
| 18 | | 3 | T2 | 13 | NP | 3 | T2 | 0 | 0 | 0 | 0 | φ | 0 | 0 | φ | 0 |
| 19 | | 3 | T2 | 15 | NP | 3 | T2 | φ | 0 | 0 | [symbol] | φ | 0 | 0 | 0 | 0 |
| 20 | | 3 | T4 | 7 | NP | 3 | T4 | 0 | 0 | 0 | [symbol] | 0 | φ | 0 | 0 | 0 |
| 21 | | 3 | T2 | 23 | NP | 3 | T2 | 0 | 0 | 0 | [symbol] | 0 | φ | 0 | 0 | 0 |
| 22 | | 3 | T3 | 19 | NP | 3 | T3 | X | 0 | 0 | 0 | 0 | 0 | φ | 0 | 0 |
| 23 | | 3 | T3 | 45 | NP | 3 | T3 | 0 | 0 | 0 | 0 | 0 | 0 | 0 | 0 | 0 |
| 24 | | 3 | T3 | 11 | NP | 3 | T3 | 0 | 0 | 0 | 0 | 0 | 0 | 0 | X | 0 |
| 25 | | 3 | T3 | 11 | NP | 3 | T3 | 0 | 0 | 0 | 0 | φ | 0 | 0 | 0 | 0 |
| 26 | | 3 | T3 | 18 | NP | 3 | T3 | 0 | 0 | 0 | 0 | 0 | 0 | 0 | 0 | 0 |
| 27 | | 3 | T3 | 16 | NP | 3 | T3 | 0 | 0 | 0 | 0 | 0 | 0 | 0 | 0 | 0 |
| 28 | | 3 | T3 | 22 | NP | 3 | T3 | 0 | 0 | 0 | 0 | 0 | 0 | φ | 0 | 0 |
| Voided urine samples* | | | | | | | | | | | | | | | | |
| 1 | | 2 | Ta | 55 | P | 1 | Ta | 0 | 0 | 0 | S | X | 0 | 0 | S | 0 |
| 2 | | | To | 60 | P | 2 | Ta | 0 | 0 | [symbol] | 0 | 0 | 0 | [symbol] | 0 | 0 |
| 3 | | 2 | Ta | 140 | P | 2 | Ta | φ | 0 | X | φ | 0 | X | 0 | 0 | φ |
| 4 | P | 2 | Ta | 149 | P | 2 | Ta | 0 | 0 | 0 | 0 | 0 | 0 | 0 | 0 | 0 |
| 5 | P | 2 | Ta | 0 | P | 2 | Ta | 0 | 0 | 0 | 0 | 0 | 0 | 0 | 0 | 0 |
| 6 | | | To | 15 | P | 2 | Ta | 0 | 0 | 0 | 0 | φ | 0 | 0 | 0 | 0 |
| 7 | | | To | 6 | P | 2 | T2 | 0 | 0 | X | 0 | X | X | 0 | X | φ |
| 8 | P | 2 | Ta | 8.5 | P | 2 | T1 | 0 | 0 | 0 | 0 | 0 | 0 | 0 | 0 | 0 |
| 9 | NP | 3 | T3a | 25 | NP | 3 | T1 | φ | φ | φ | 0 | X | φ | 0 | 0 | 0 |
| 10 | | 3 | Tis | 0.6 | NP | 3 | T2 | 0 | 0 | [symbol] | 0 | 0 | 0 | 0 | [symbol] | 0 |
| 11 | NP | 3 | T2 | 2.5 | NP | 3 | T2 | 0 | [symbol] | 0 | 0 | 0 | 0 | [symbol] | [symbol] | 0 |
| 12 | NP | 3 | T2 | 0 | NP | 3 | T2 | 0 | 0 | 0 | 0 | 0 | 0 | 0 | X | 0 |
| 13 | | | To | 1 | NP | 3 | T2 | 0 | X | 0 | φ | X | 0 | 0 | φ | 0 |
| 14 | NP | 3 | T3b | 1 | NP | 3 | T1 | 0 | 0 | [symbol] | 0 | φ | 0 | 0 | 0 | 0 |
| 15 | | 3 | Tis | 1 | NP | 3 | T2 | 0 | [symbol] | 0 | 0 | 0 | 0 | 0 | 0 | 0 |
| 16 | NP | 3 | T4 | 4 | NP | 3 | T2 | [symbol] | 0 | 0 | 0 | 0 | 0 | 0 | 0 | 0 |
| 17 | | | To | 3 | NP | 3 | T2 | φ | 0 | 0 | 0 | 0 | φ | [symbol] | X | φ |
| 18 | | 3 | Ta | 120 | NP | 3 | T3 | 0 | 0 | 0 | 0 | 0 | 0 | 0 | 0 | 0 |
| 19 | | | To | 0.6 | NP | 3 | T2 | 0 | 0 | 0 | 0 | 0 | 0 | 0 | 0 | 0 |
| 20 | NP | 3 | T1 | 1.6 | NP | 3 | T1 | 0 | 0 | 0 | 0 | 0 | 0 | 0 | 0 | φ |
| 21 | NP | 3 | T3a | 1 | NP | 3 | T2 | 0 | 0 | 0 | 0 | 0 | 0 | 0 | 0 | 0 |
| 22 | | 3 | Tis | 0.6 | NP | 3 | T1 | 0 | 0 | 0 | 0 | X | 0 | 0 | 0 | φ |
| 23 | P | 2 | T1 | | | | | 0 | [symbol] | 0 | [symbol] | 0 | 0 | φ | [symbol] | 0 |
| 24 | | | To | 2 | | Tis | Tis | 0 | 0 | X | 0 | 0 | 0 | 0 | 0 | 0 |
| 25 | NP | 3 | T3 | | | | | 0 | 0 | 0 | 0 | 0 | 0 | 0 | 0 | φ |

•: LOH;
0: no LOH;
φ: non-informative;
X: no reaction;
S: shortening.
*Urine samples for this analysis were not obtained from the same patients as bladder tumor samples

TABLE 16
Frequency of LOH on five distinct regions of chromosome 16 identified on
tumor and voided urine samples of patients with urinary bladder dysplasia.

| | | | | | Voided urine samples | |
|---|---|---|---|---|---|---|
| | | | Tumor samples | | Frequency | |
| | | | Frequency of LOH (%) | | of LOH (%) | |
| | | Deleted | Individual | | Individual | |
| Region | Marker | Region (cM) | marker | Region | marker | Region |
| p13.3 | D16S418 | 1.2 | 7.1 | 17.9 | 4.0 | 16.0 |
| | D16S513 | | 10.7 | | 8.0 | |
| | D16S406 | | 3.6 | | 4.0 | |
| p13.1 | D16S748 | 12.9 | 3.6 | 28.6 | 4.0 | 20.0 |
| | D16S500 | | 17.9 | | 8.0 | |
| | D16S287 | | 14.3 | | 8.0 | |
| q12.1 | D16S409 | 24.0 | 14.3 | 46.4 | 8.0 | 32.0 |
| | D16S541 | | 28.6 | | 20.0 | |
| | D16S415 | | 10.7 | | 4.0 | |
| | D16S514 | | 0.0 | | 8.0 | |
| q22.1 | D16S496 | 5.4 | 3.6 | 14.3 | 4.0 | 28.0 |
| | D16S512 | | 3.6 | | 12.0 | |
| | D16S515 | | 10.7 | | 12.0 | |
| q24 | D16S507 | 5.9 | 21.4 | 21.4 | 4.0 | 4.0 |
| | D16S505 | | 0.0 | | 0.0 | |
| | D16S511 | 17.4 | 0.0 | 10.7 | 0.0 | 20.0 |
| | D16S402 | | 0.0 | | 12.0 | |
| | D16S520 | | 7.1 | | 12.0 | |
| | D16S413 | | 3.6 | | 0.0 | |

Analysis of contig and sequencing data spanning the deleted regions of chromosome 16

The analysis of available contig and sequencing data spanning the deleted regions of chromosome 16 is summarized in FIG. 38. The five deleted regions of chromosome 16 contain 88 known genes, some of them with potential tumor suppressor gene activities. In addition multiple ESTs were assigned to individual deleted regions identifying several smaller gene-rich areas. The two most frequently deleted regions mapping to 16q12.1 and q22.1 contained several smaller areas with particularly high densities of ESTs and of known genes with putative tumor suppressor activities, further supporting the concept of their potential pathogenetic relevance for bladder carcinogenesis.

### Example 12

### Genetic Mapping and DNA Sequence-Based Analysis of Deleted Regions on Chromosome 13 Involved in Progression of Bladder Cancer from Occult Preneoplastic Conditions to Invasive Disease, with Particular Emphasis on the Role of the RB Gene

An example of deletion map for chromosome 13 generated by whole-organ histologic and genetic mapping is provided in Figure X. Chromosome 13 was selected for presentation as it contains a model tumor suppressor gene, the RB gene. The locus was originally mapped by genetic linkage in a familial form of retinoblastoma and the target RB gene was identified by the positional cloning strategy. The RB gene was subsequently proven to play a major role in the development of many sporadic human cancers including bladder carcinoma. The inactivation of RB in human cancers follow in general a concept of double hit theory and recent studies have indicated that it is involved in early preneoplastic phases of human bladder neoplasia. Therefore, a plaque-like expansion of allelic losses involving large areas of bladder mucosa identified by the hypervariable DNA markers mapping to within and around the RB gene will validate our approach and permit a reasonable speculation that the identification of similar alterations in novel loci may guide us to unknown tumor suppresser genes involved in early phases of bladder neoplasia.

Whole-Organ Histologic and Genetic Mapping.

Radical cystectomy specimens from eight patients with previously untreated sporadic high grade invasive transitional cell carcinoma (TCC) of the bladder were used for the whole-organ histologic and genetic mapping as previously described (Chaturvedi et al, 1997; Czerniak et al, 1999; and 2000). All patients were men and their ages ranged from 47 to 78 years (mean =66.4±11.9 years SD).

In brief, each fresh cystectomy specimen was opened longitudinally along the anterior wall of the bladder and pinned down to a paraffin block. The entire mucosa was than divided into 1×2 cm rectangular samples and evaluated microscopically on frozen sections. The tissue of interest was microdissected from the frozen block and used to prepare a urothelial cell suspension by mechanically scrapping the urothelial mucosa or gentle shaking invasive tumor samples. Only those specimens that yielded more than 90% of microscopically recognizable intact urothelial or tumor cells in each sample were accepted for the study and used for DNA extraction. This procedure provided 49, 39, 65, 42, and 39 DNA samples from each cystectomy specimen that corresponded to microscopically identified intraurothelial precursor conditions or invasive carcinoma. As a control, DNA extracted from the peripheral blood lymphocytes and/or from normal tissue in the resected specimen of each patient was used.

The intraurothelial precancerous changes were classified as mild, moderate, and severe dysplasia or carcinoma in situ. The tumors were classified according to the three-tier histologic grading system of the World Health Organization (Mostofi, 1999). The growth pattern of papillary versus nonpapillary or solid tumors and the depth of invasion were also recorded. In seven of the eight cystectomy specimens, a single focus of grade 3 nonpapillary urothelial carcinoma invaded the muscularis propria and was accompanied by extensive precancerous lesions ranging from mild dysplasia to carcinoma in situ. In the remaining case, multiple foci of carcinoma were present. One focus represented a grade 3 nonpapillary urothelial carcinoma with transmural invasion of the bladder wall and involvement of perivesical adipose tissue. Two additional foci of carcinoma represented grade 3 papillary urothelial carcinoma without invasion. Like the other seven cases, this case exhibited changes ranging from mild dysplasia to carcinoma in situ over extensive areas of the urinary bladder mucosa. The results of microscopic evaluation of individual samples from five cystectomy specimens were recorded and stored in a computer database as histologic maps.

Tumors and Voided Urine Samples of Patients with Bladder Cancer.

The markers of chromosome 5 that were identified as significantly altered by the whole histologic and genetic mapping were tested in 37 tumor and 29 voided urine samples. The tumors were classified according to the three-tier histologic grading system of the World Health Organization (Mostofi, 1999). The growth pattern, tumor grade and depth of invasion were also recorded. Levels of invasion were recorded according to the TNM staging system (Sobin et al, 1997). DNA was extracted from individual bladder tumors and sediments of voided urine samples as previously described (Chaturvedi et al, 1997). For controls, DNA was also extracted from the peripheral blood lymphocytes and/or normal tissue in the resected specimens from each patient.

Microsatellites.

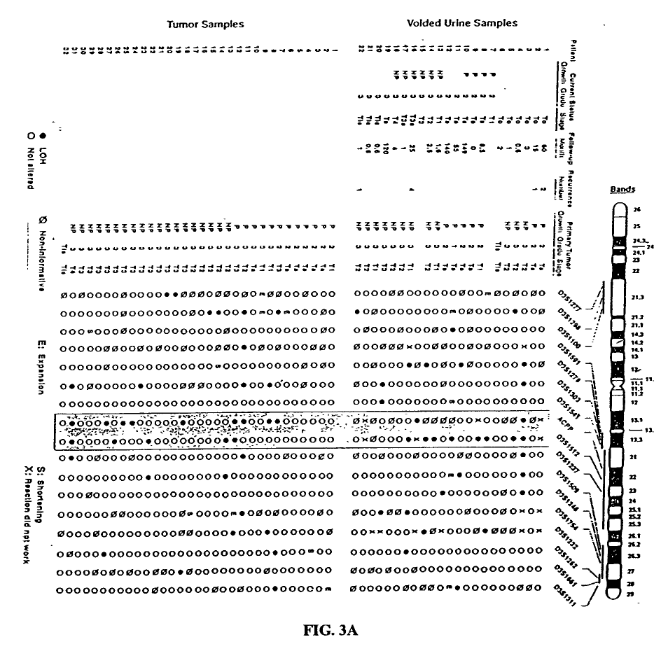

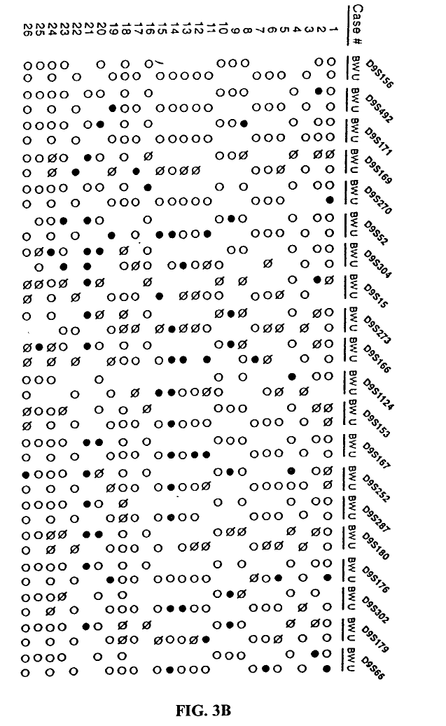

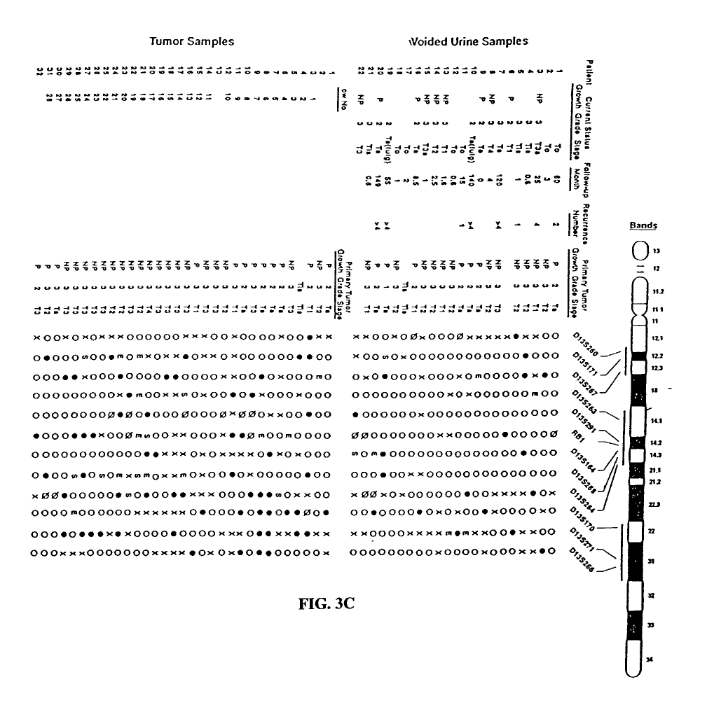

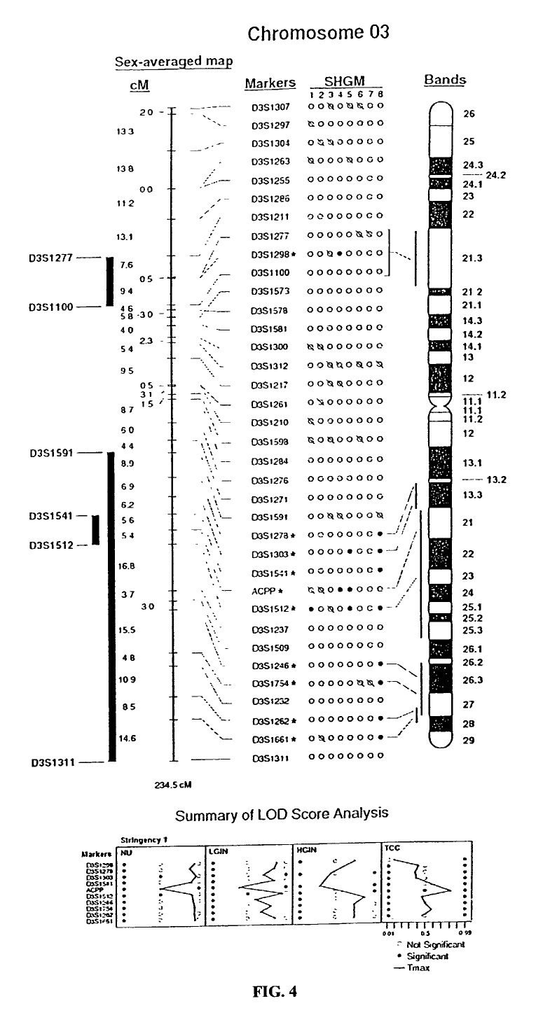

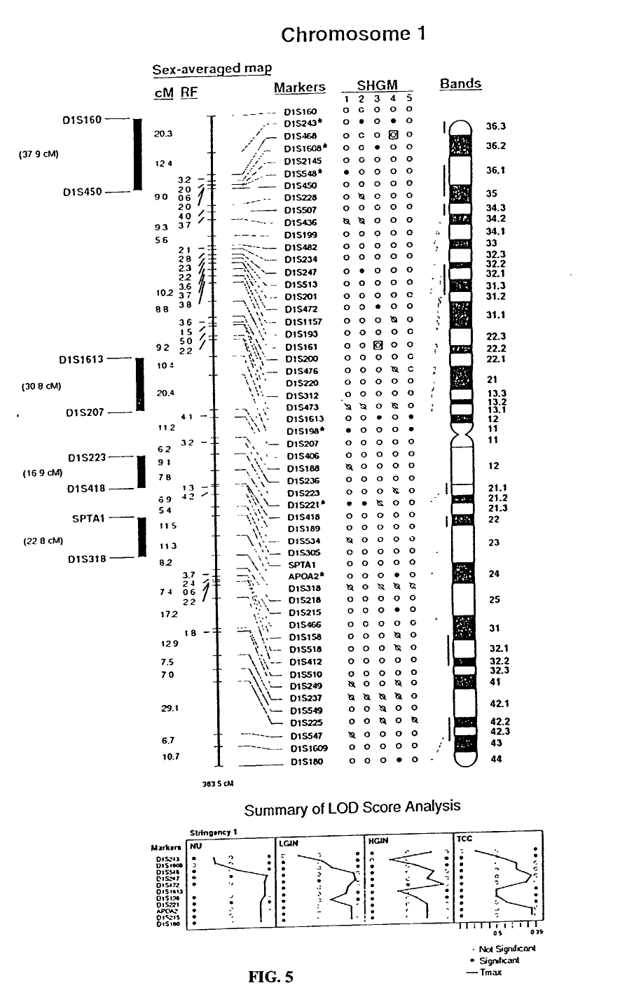

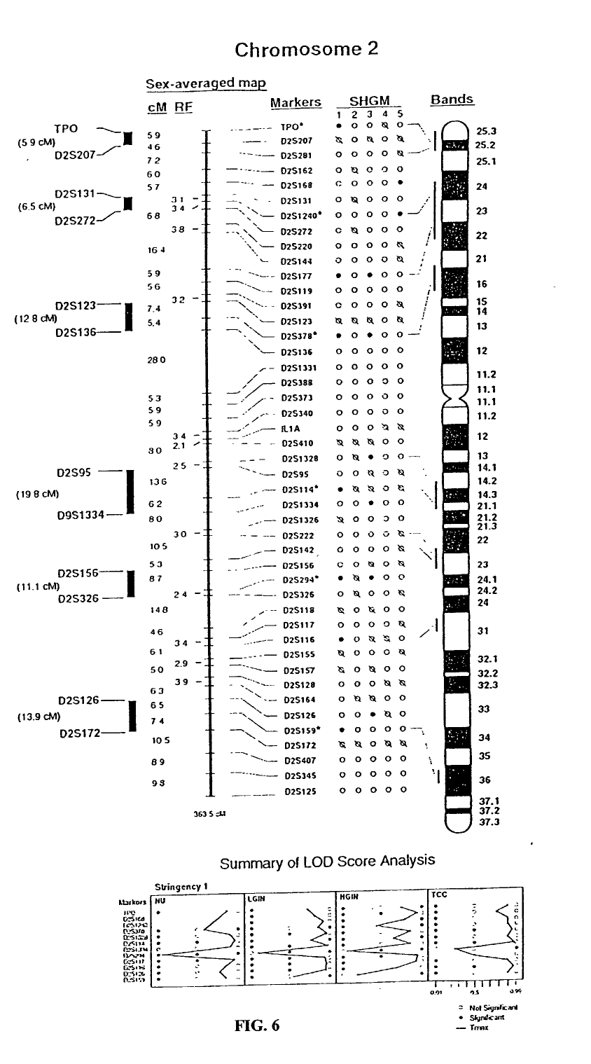

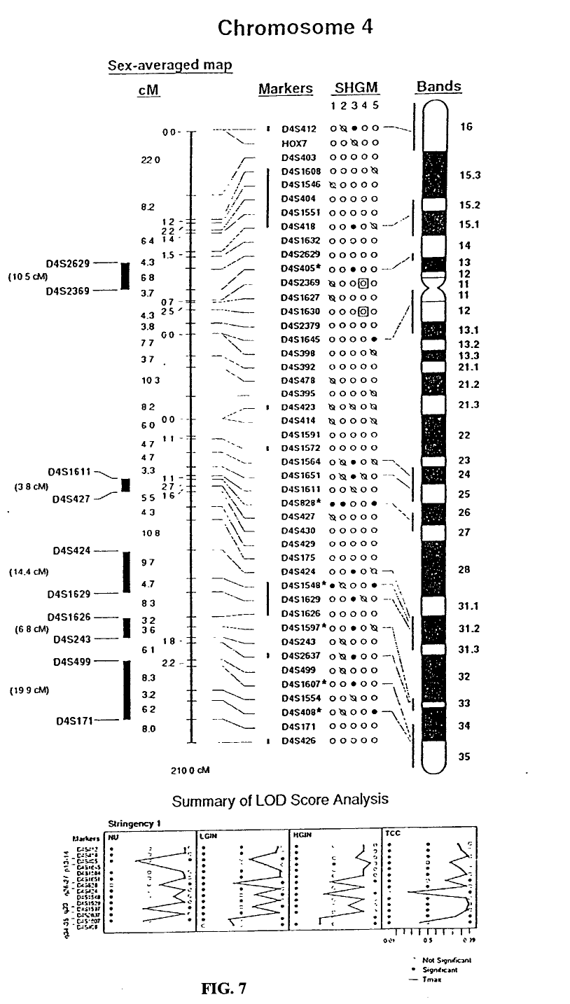

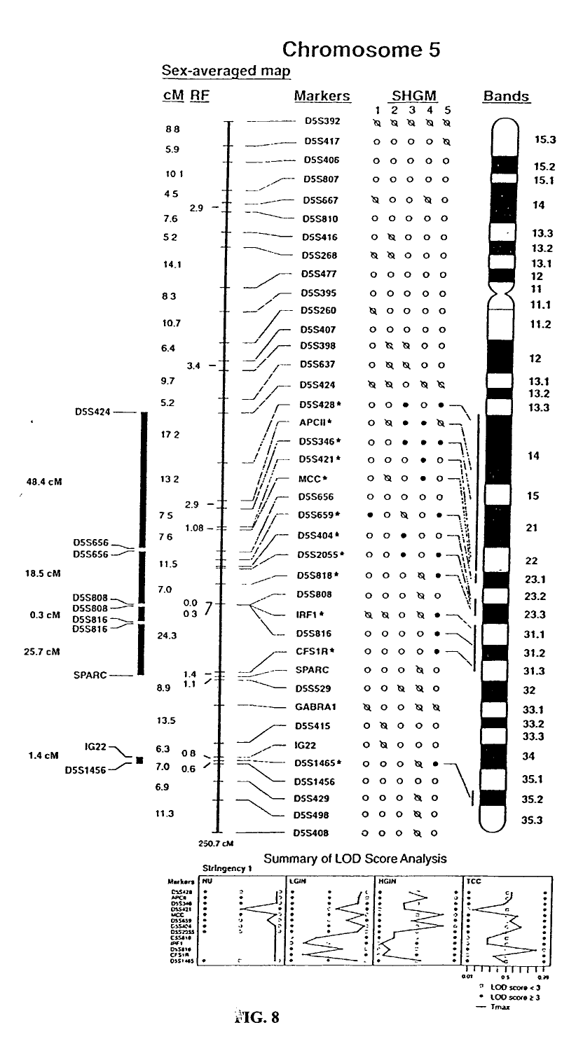

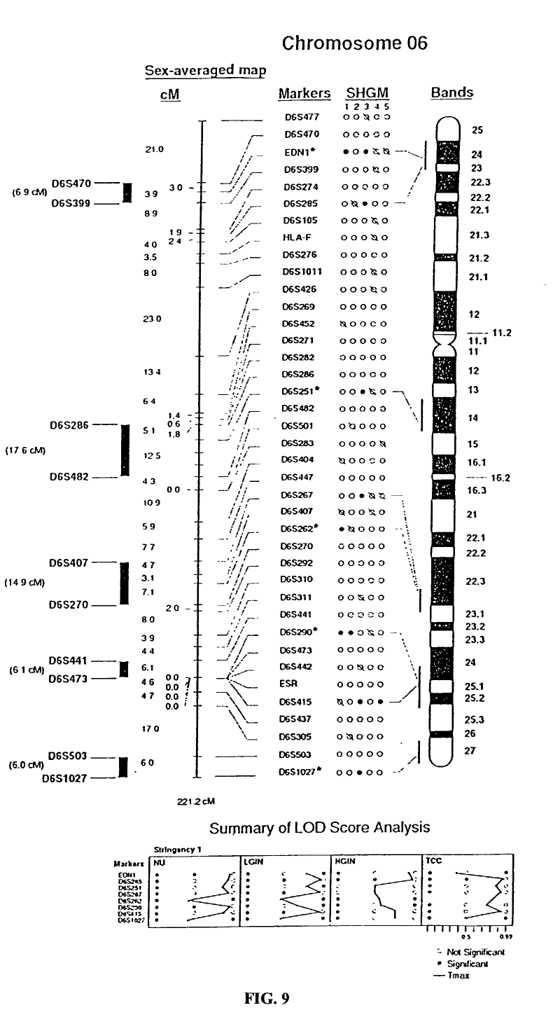

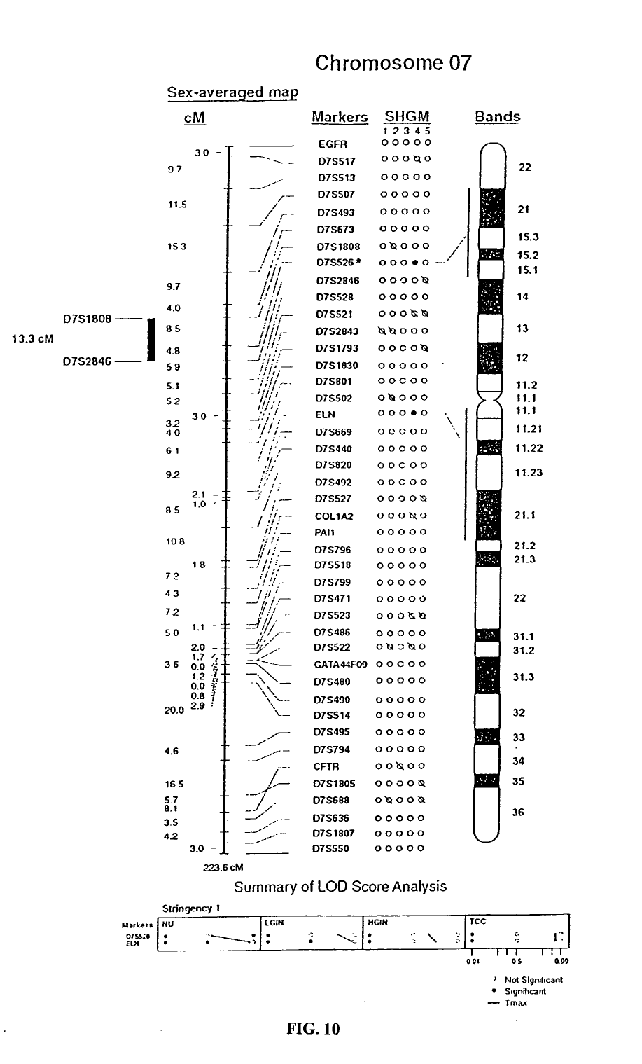

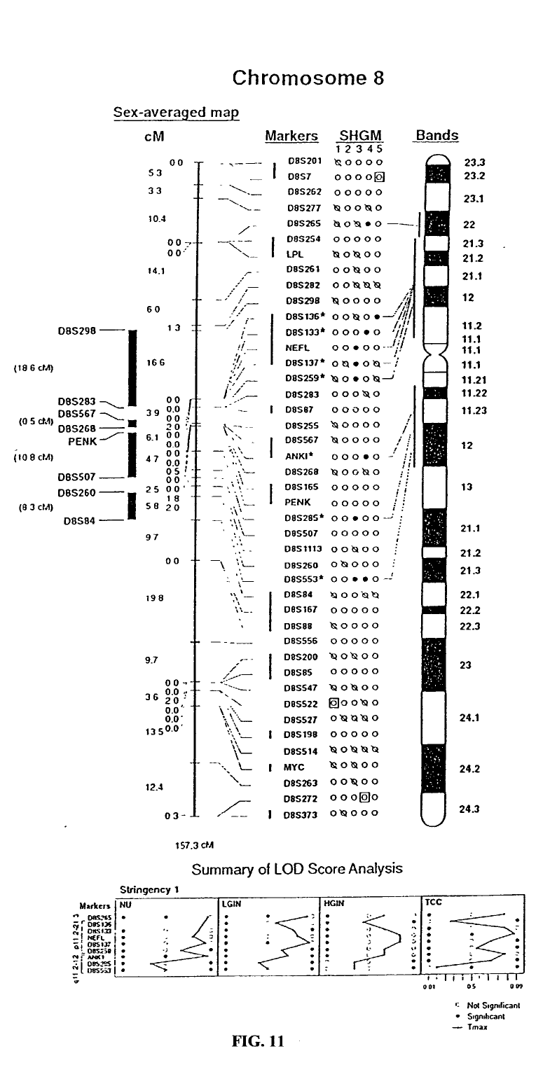

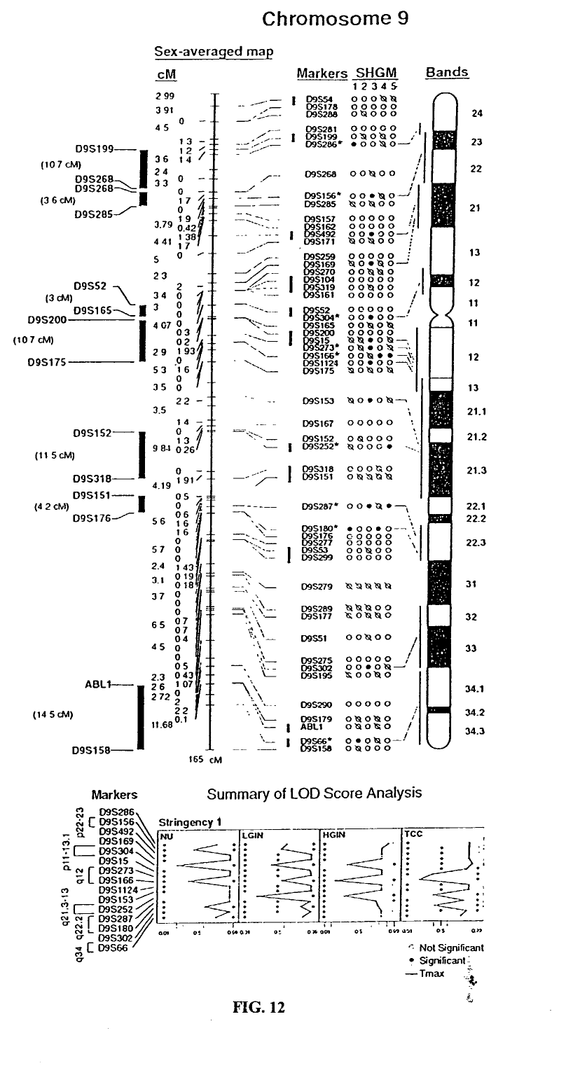

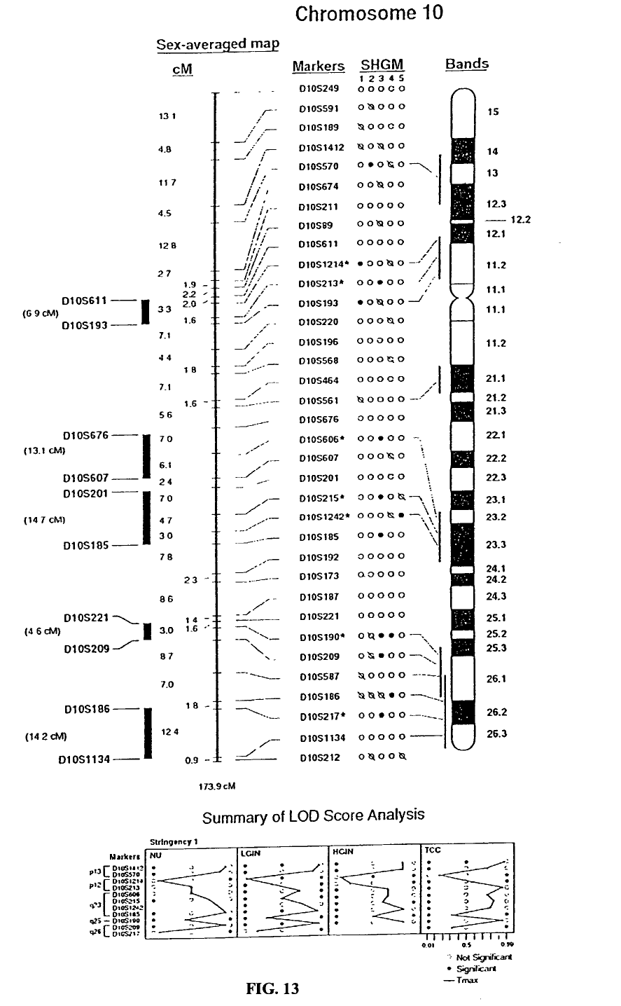

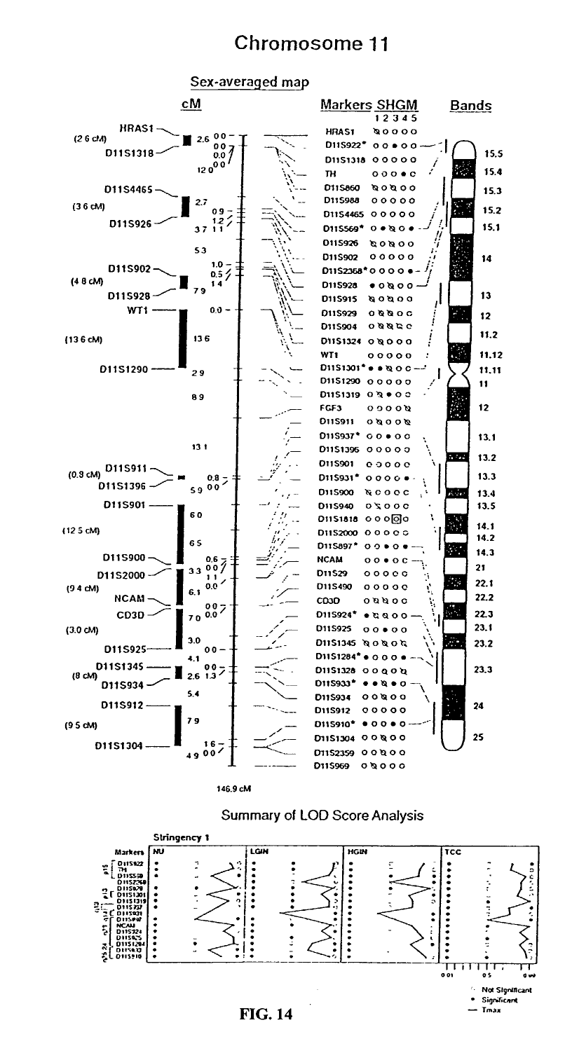

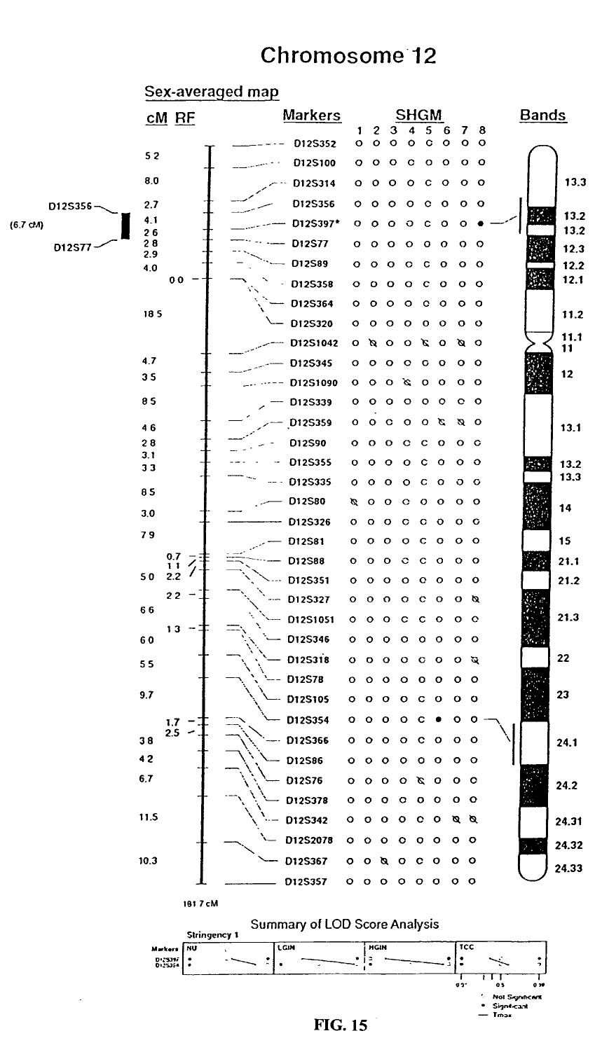

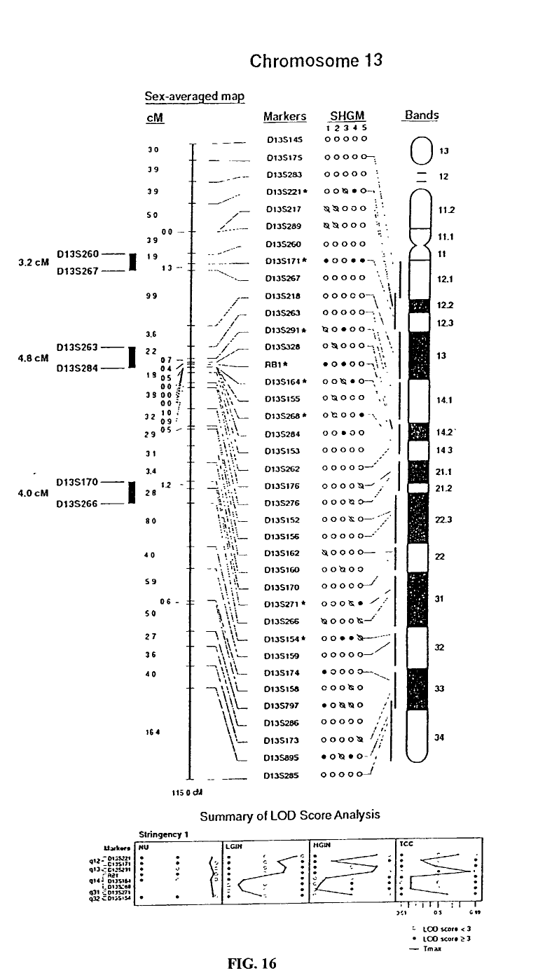

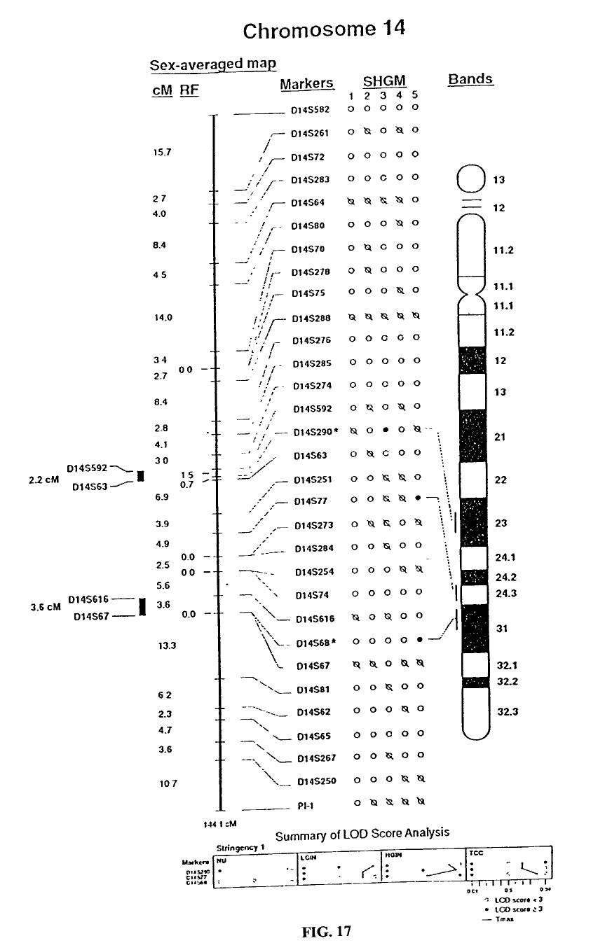

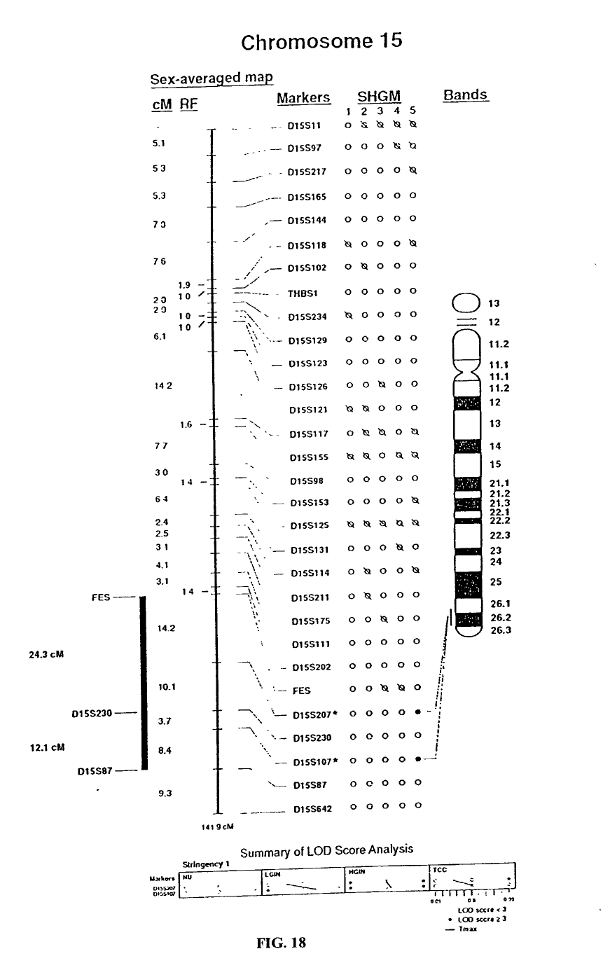

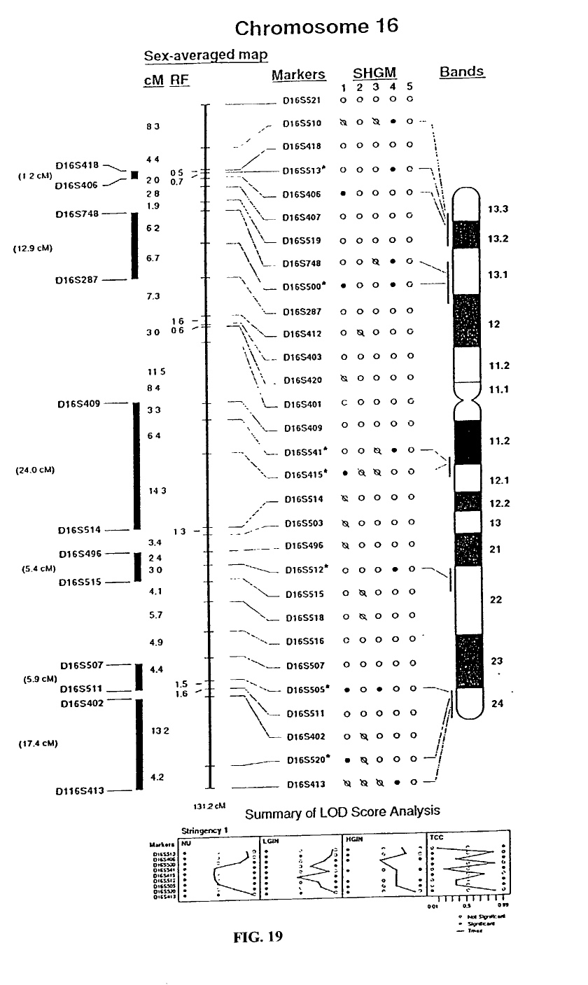

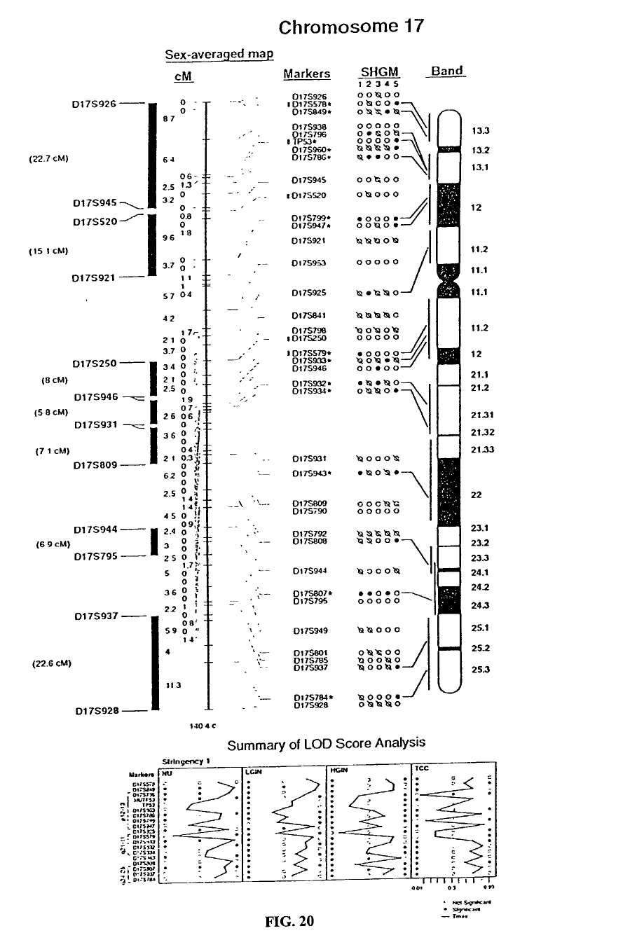

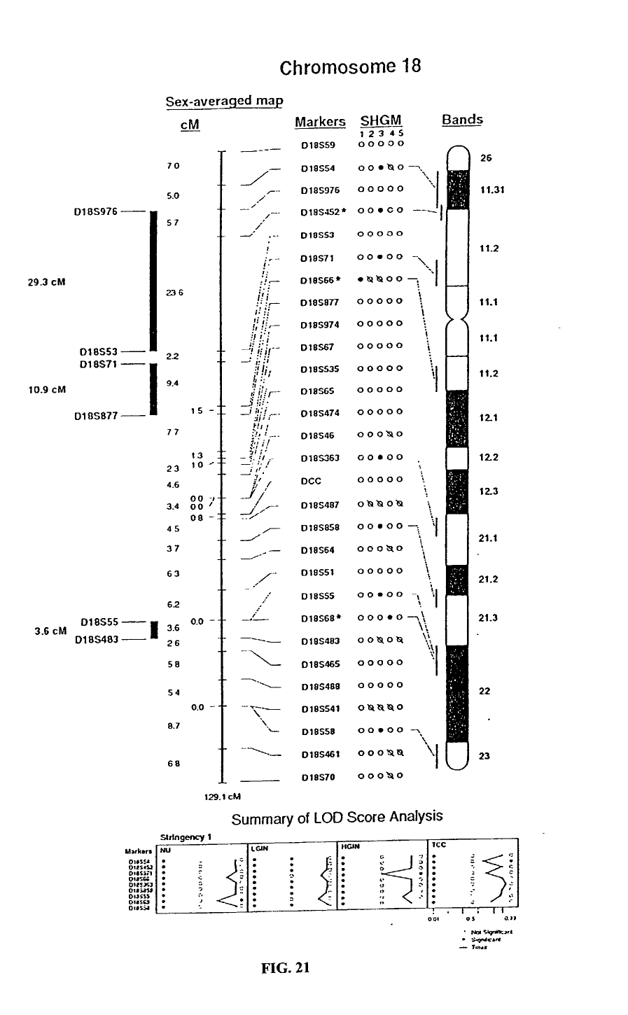

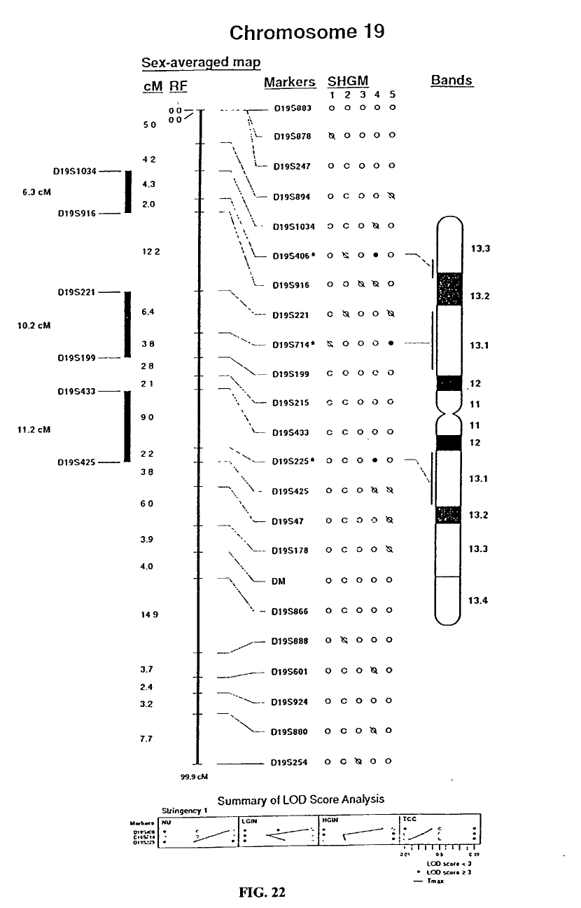

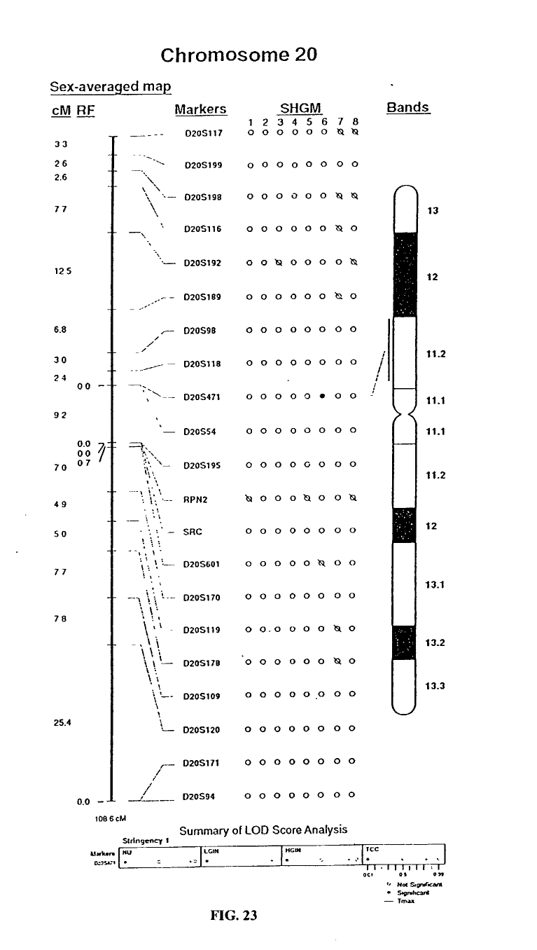

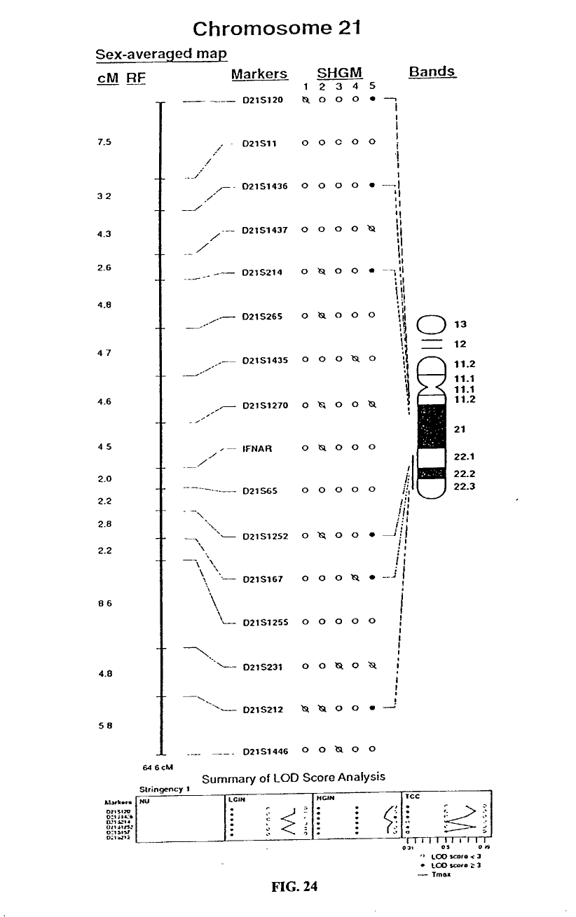

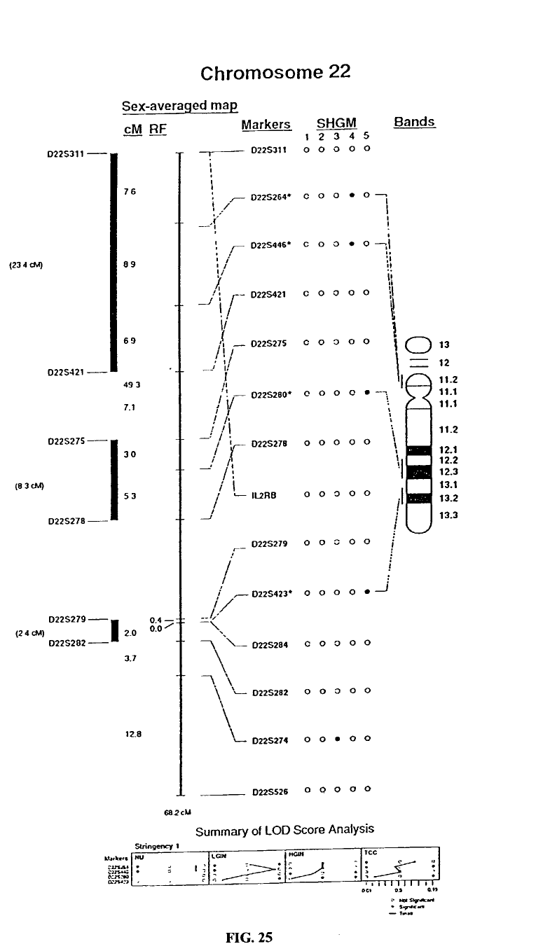

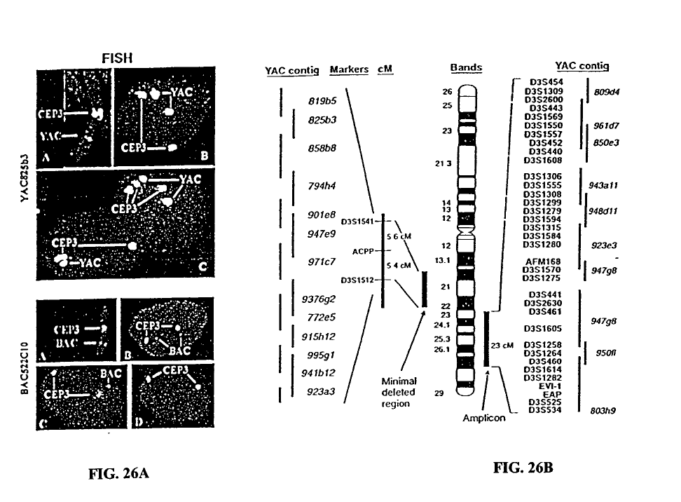

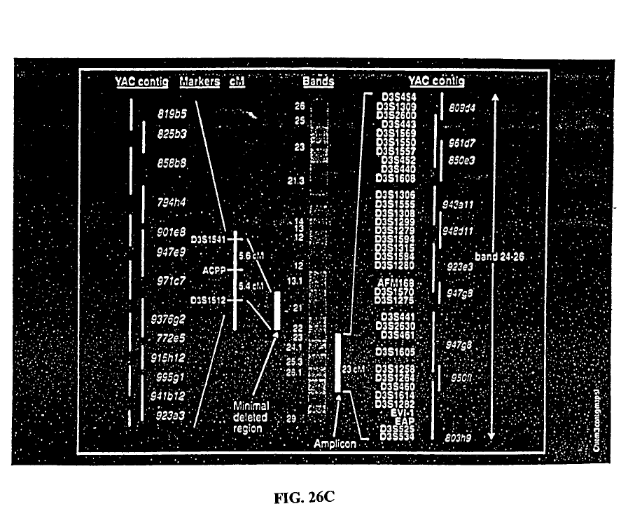

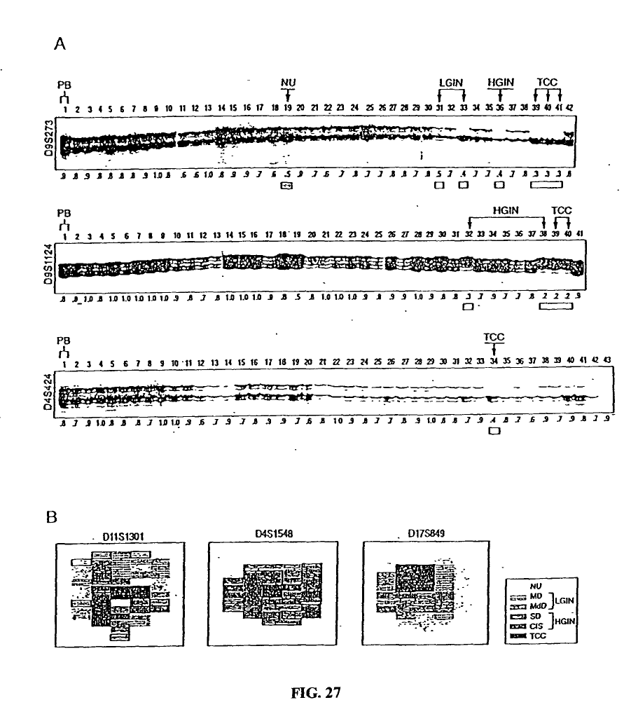

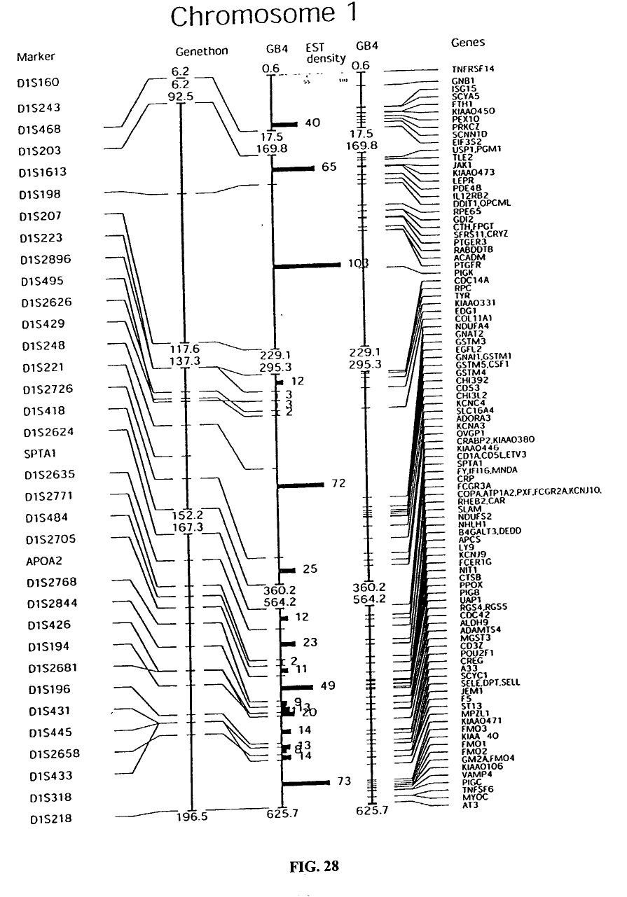

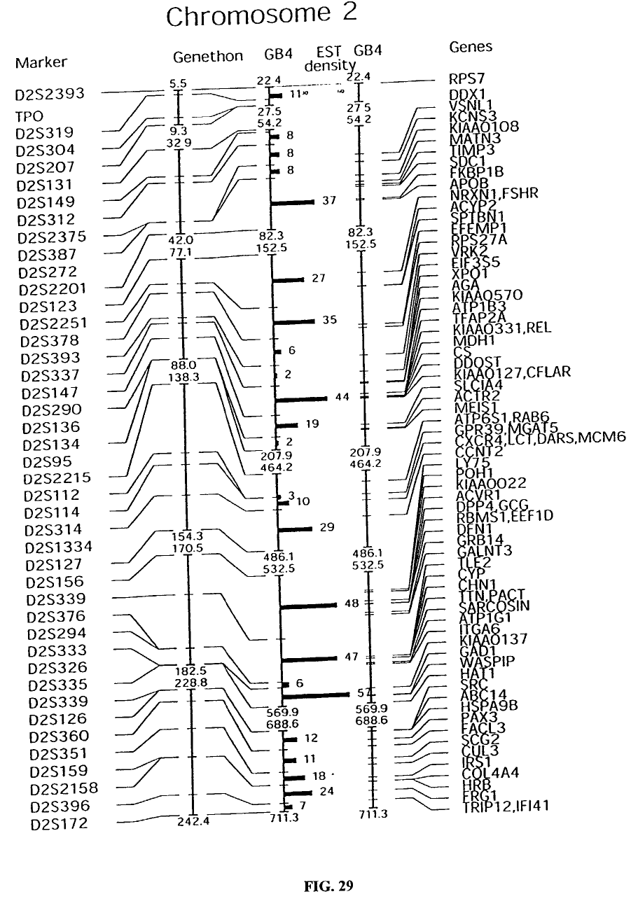

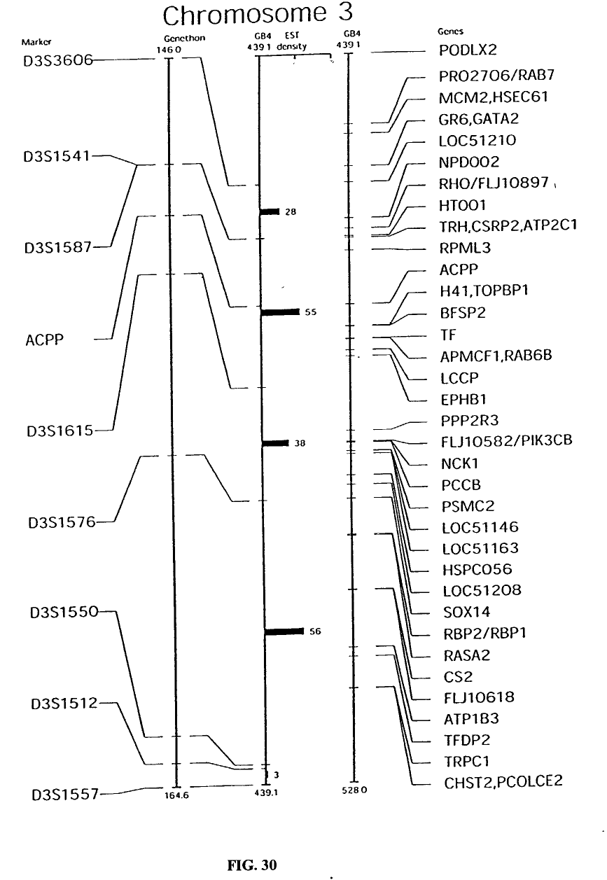

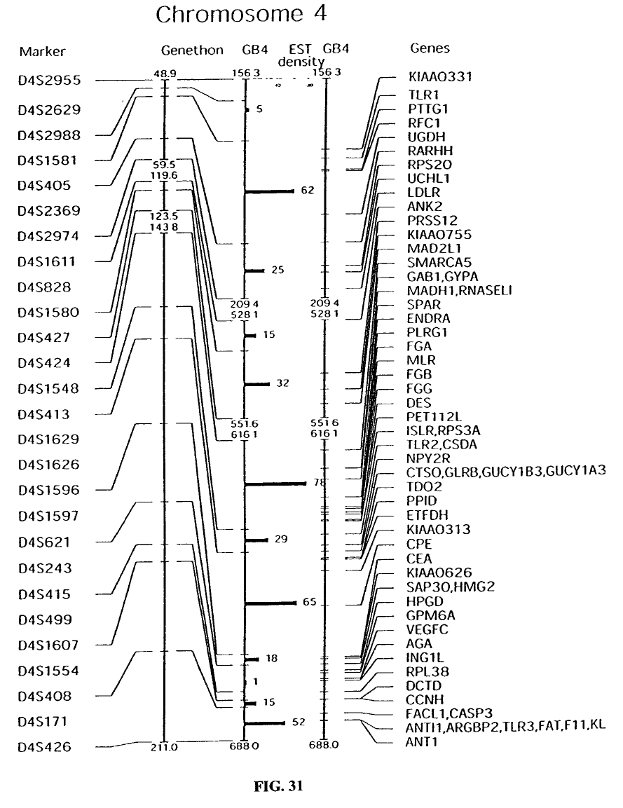

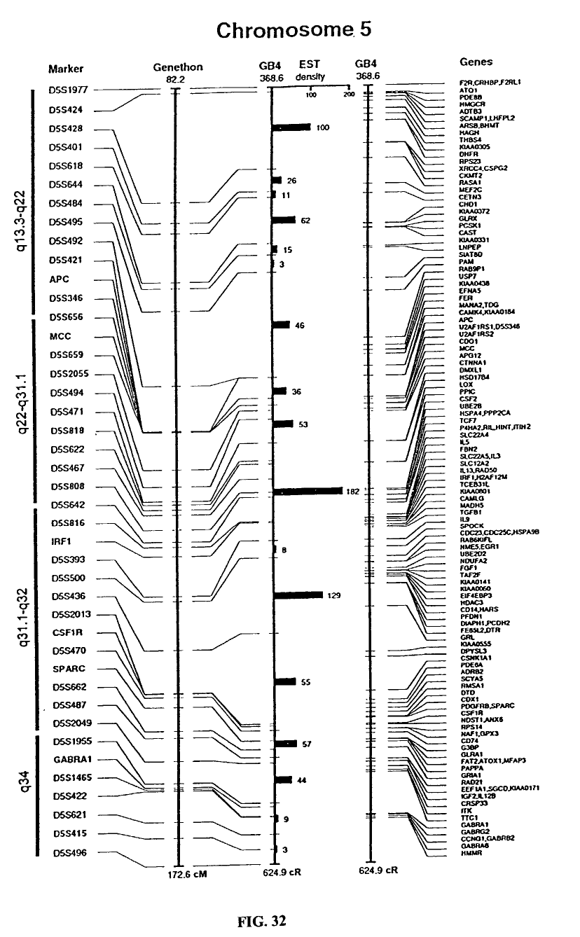

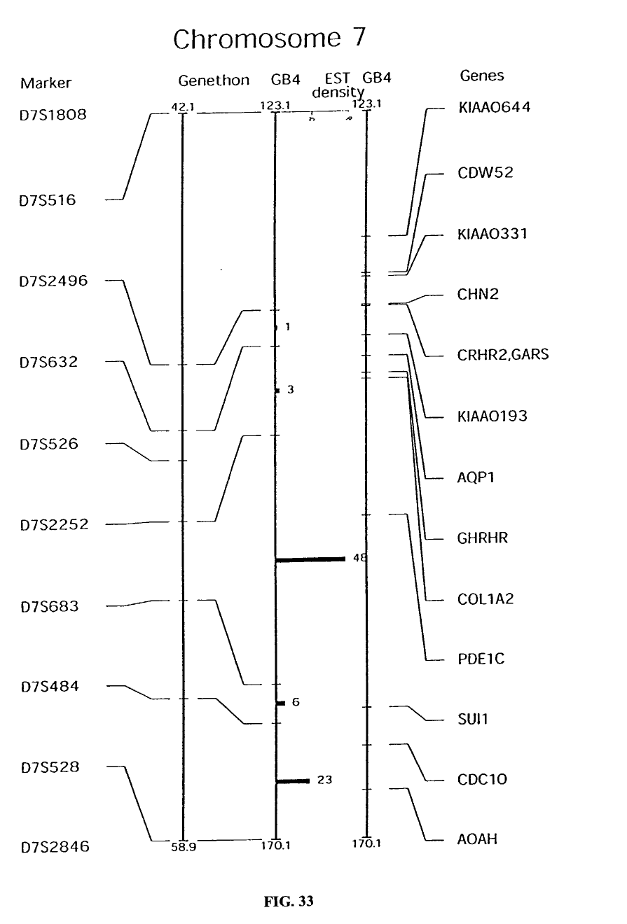

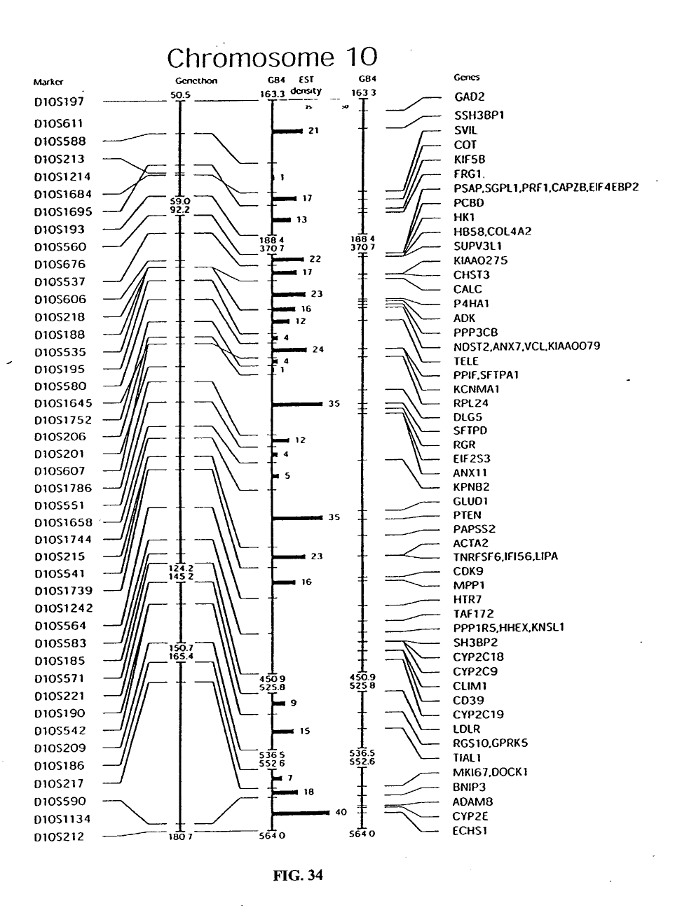

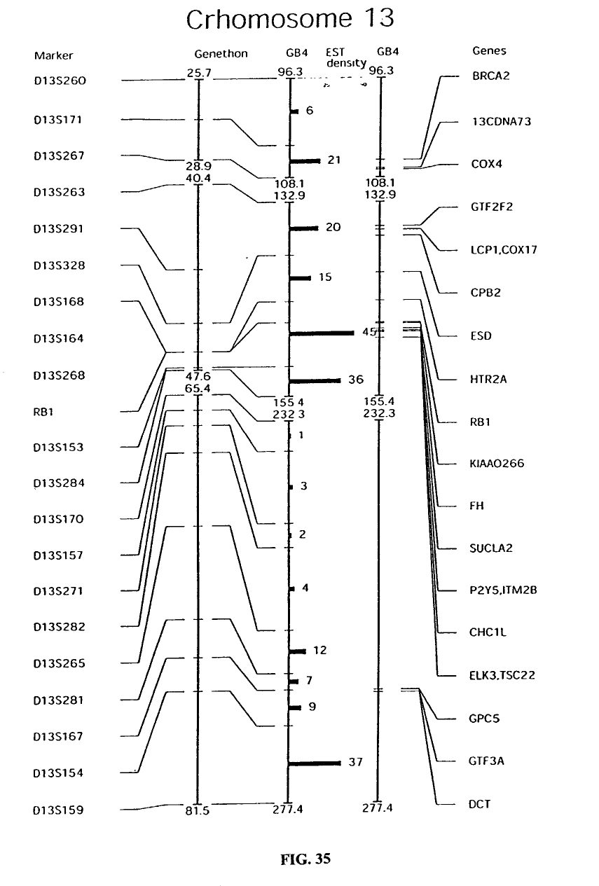

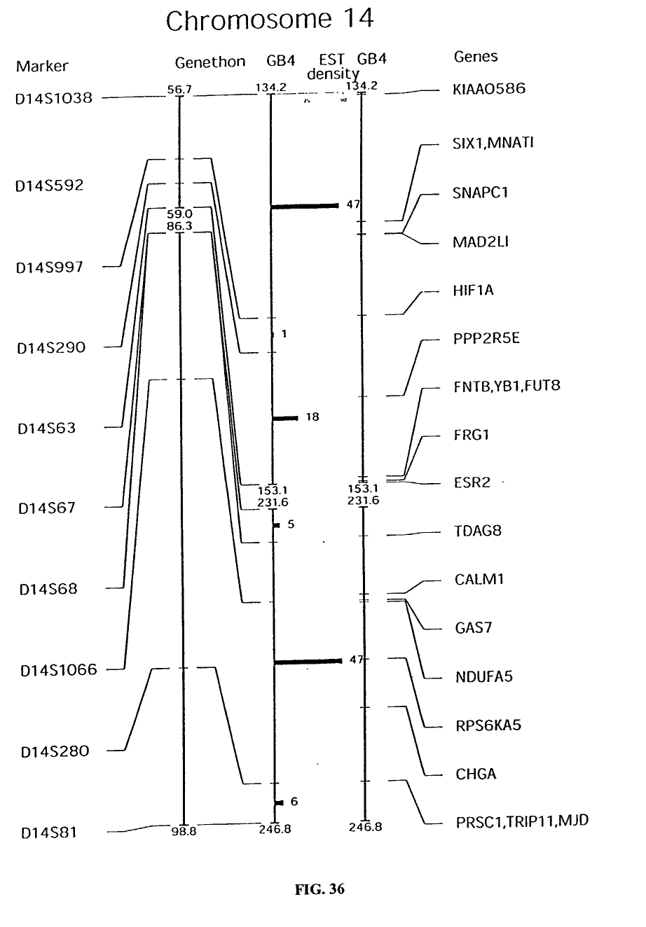

A set of primers for 787 microsatellite loci on chromosomes 1-22 based on integrated sex averaged microsatellite map from Genethon (version March 1966) and updated by Cooperative Human Linkage Center (version 4.0) was obtained from Research Genetics (Huntsville, Ala., USA). The markers selected for testing exhibited high levels of heterozygosity and uniform distribution covering all regions of tested chromosomes. The allelic patterns of markers were resolved on 6% polyacrylamide gels after their amplification using polymerase chain reaction as previously described (Chaturvedi et al, 1997). A minimum 50% reduction in signal intensity was required to be considered as evidence of LOH. Tests with questionable results were repeated. In such cases, the densitometric measurements were performed to ensure objective reading of the data. A small proportion of markers showed expansion or shortening of their repetitive sequences that involved individual mucosal samples and could not be statistically related to the development and progression of urothelial neoplasia. The differences in length of the repetitive sequences identified were considered as sporadic random events were related to overall genomic intensity associated with the malfunctioning DNA repair genes. The markers showing shortening or expansion are identified on individual chromosomal maps but since they showed no relationship to the progression of bladder neoplasia they were not included in the final data analysis shown in FIGS. 2-4.

Genotyping with SNP'S.

The SNP sites were genotyped using the pyrosequencing methods. In brief, genomic DNA fragments containing SNP's were amplified by PCR with one of each primer pair covalently coupled to biotin. Single stranded DNA was isolated by streptavidin-coated paramagnetic beads (Dynalbeads M280; Dynal, Norway). Allelotyping of SNP's was performed using an automative Pyrosequencing instrument PSQ96 (Pyrosequencing AB). The sequencing reaction mixture contained the single-stranded DNA with sequencing primer annealed, exonuclease-deficient DNA polymerase apyrase, purified luciferase, ATP sulfurylase, adenosine 5′-phophosulfate and luciferin. The sequence was determined from the measured signal output of light upon nucleotide incorporation. The resulting peaks were analyzed using Pyrosequencing software (Pyrosequencing AB). A minimum of 50% of signal intensity reduction from one of the polymorphic nucleotides was used to identify a hapotype (allelic loss). Allelotyping of SNP's was performed in the three sequential steps. Initially, all selected SNP's of normal genomic DNA were sequenced. In the next steps, those SNP's which exhibited polymorphism were tested on paired normal-invasive tumor DNA samples of the same patient. In the final step, those SNP's which showed allelic loss were tested on all mucosal samples of the same cystectomy specimen. The distribution of clonal allelic losses of each SNP's was subsequently superimposed over the histologic map of the entire organ and integrated with the distribution patterns of clonal allelic losses identified by the hypervariable DNA markers.

Statistical Analysis of Data.

The data were organized and analyzed as previously described (Chaturvedi et al, 1997; Czerniak et al, 1999; and 2000). In brief, the information on LOH in individual loci was entered into the data files and superimposed over the histologic maps. Initial data consisted of chromosomal vectors with a list of LOH in individual loci and coordinates for locations of mucosal samples, which could be used to plot the distribution of LOH to microscopically classified urothelial changes. By superimposing plots of LOH over the histologic maps, we identified the areas of bladder mucosa with altered markers and analyzed their relationship to intraurothelial precursor conditions and invasive cancer. Three-dimensional displays of LOH in relation to the progression of neoplasia from precursor intraurothelial conditions to invasive cancer were generated and initially analyzed by the nearest-neighbor algorithm (Hartigan, 1975).

The relationship between altered markers and the progression of urothelial neoplasia from precursor conditions to invasive cancer was tested by a binomial maximum likelihood analysis, and the significance of the relationship was expressed as LOD score (Ott, 1991). We chose LOD scores because they represent a powerful method of likelihood analysis that can verify the statistical significance of the relationship among patterns of sequential events. The LOD scores were applied in their generic mathematical sense as likelihood tests of events, not as in their common use to test the linkage in familial disorders with meiotic segregation of the phenotype at a recombination fraction θ=0.5. In sporadic cancer when microscopically defined stages of cancer progression are used as standards of sequential events and there is a mitotic transmission of the phenotype, the null hypothesis is more appropriately verified at a recombination factor differing from 0.5. Hence, cumulated LOD scores were calculated at variable θ=0.01, 0.5, and 0.99. A pattern of LOD scores ≧3 at θ=0.01 or θ=0.99 and LOD scores <3 at θ=0.5 for the same marker was considered significant. The strongest association between altered marker and neoplasia was when a LOD score was ≦3 and θ=0.99 and 0.5 and <3 at θ=0.01. Stringency 1 designated LOD scores for specific stages of neoplasia. Stringency 2 designated LOD scores for progression to higher stages of neoplasia. The analysis of relationship among LOH in individual loci and various clinico-pathological parameters of tumors and of voided urine samples was tested by Gehan's generalized Wilcoxon, and log-rank tests (p≦0.05 was considered significant).

Finally, the patterns of LOH distributions in relation to progression of neoplasia were clustered using the hierarchical command in SPSS (SPSS, Inc, Chicago Ill.) and compared with the results of the binomial maximum likelihood analysis. The Hamann distance measure was used to evaluate the degree of agreement to match clusters using the total number of matches in each category of samples i.e. NU, LGIN, HGIN, and TCC minus the number of non-matches normalized by the total number of samples analyzed. This produced a measure that varied from −1 (complete disagreement) to 1 (complete agreement).

Whole-organ histologic and genetic mapping studies identified five clusters of allelic losses mapping to distinct regions of chromosome 13. The deleted regions defined by the nearest non-altered flanking markers and their predicted size in centimorgans (cM) were as follows: 13q12.2(D13S175-DBS289,12.8cM),13q12.3(D13260-DB267,3.2cM),13q14(D13S263 BS153,3.3cM),13q14(DBS284-D13S276,4.7cM),13q21(D13S170- D3S159,16cM). As anticipated, a deleted segment mapping to 13q14 flanked by D13S263 and D13S153 which contained the RB gene showed clonal allelic losses involving large areas of bladder mucosa encompassing not only invasive cancer and adjacent severe dysplasia or carcinoma in situ but also areas of low to moderate dysplasia focally extending to areas of microscopically normal urothelium. Moreover, sequential allelic losses involving markers located within and around the RB gene were documented in progression from low to high-grade intraurothelial neoplasia and ultimately to invasive cancer.

An additional cluster of allelic losses flanked by D13S284 and D13S276 was identified within the 13q14 region. Clonal allelic losses in this segment were associated with the development of early in situ phases of neoplasia progressing to invasive cancer and could be synchronous or dis-synchronous with the involvement of the RB containing region. Such pattern of alterations suggests a presence of alternative target gene or genes within the 13q14 region, which are involved in early phases of bladder neoplasia. The three remaining segments of allelic losses mapping to 13q12 and 13q13 were associated with some limited clonal expansion related to the intraurothelial neoplasia, but not to invasive cancer. It is therefore, highly unlikely that they contain tumor suppressor genes playing a major role in human bladder carcinogenesis.

In order to further investigate the involvement of chromosome 13 regions identified by whole-organ histologic and genetic mapping several additional studies were performed. Since the major limitation of whole-organ histologic and genetic mapping is that these laborious studies can be performed on the limited number of cases the frequency of allelic losses in target regions of chromosomal 13 was verified on a larger number of tumor and voided urine samples of patients with bladder cancer. It turned out that allelic losses of markers mapping to the 13q14 RB gene containing region could be detected in approximately 50% of bladder tumors. Allelic losses in other chromosome 13 regions identified by whole-organ histologic and genetic mapping could be detected in less than 10% of the cases only. This confirmed that the 13q14 region containing the RB gene plays a major role in bladder carcinogenesis.

In subsequent studies we focused our attention on the pattern of RB involvement in development of urothelial neoplasia by sequencing the multiple SNP sites within and around the RB gene in all mucosal samples of cystectomy specimens. This provided more accurate deletion map of the region as compared to the map generated by the hypervariable DNA markers. When whole-organ maps of clonal allelic losses identified by SNP's were integrated with the patterns of allelic losses identified by the hypervariable DNA markers, it became evident that a loss of DNA segment spanning at least 8 Mb centered around RB may represent an incipient event in the development of bladder neoplasia. Such losses were associated with clonal expansion of abnormal urothelial cells involving large areas of bladder mucosal and were antecedent to the development of mircroscopically recognizable precursor conditions such as dysplasia. On the other hand, it turned out that the second deletion inactivating the remaining RB allele (RB 1.2) occurred later and was associated with the development of severe dysplasia/carcinoma in situ progressing to invasive TCC. In summary, these studies disclosed sequential hits within the RB gene containing region of chromosome 13 that could be assigned to specific phases of bladder neoplasia. Moreover, they provided a strong evidence for other genes mapping to the same region whose involvement is preceding the inactivation of RB.

All of the APPARATUS and/or METHODS disclosed and claimed herein can be made and executed without undue experimentation in light of the present disclosure. While the compositions and methods of this invention have been described in terms of preferred embodiments, it will be apparent to those of skill in the art that variations may be applied to the APPARATUS and/or METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

PCT Application No. WO 94/05414

U.S. Pat. No. 5,304,487

U.S. Pat. No. 5,296,375

U.S. Pat. No. 5,856,174

U.S. Pat. No. 5,837,832

U.S. Pat. No. 5,837,860

U.S. Pat. No. 5,427,910

U.S. Pat. No. 5,523,207

U.S. Pat. No. 5,861,242

U.S. Pat. No. 5,578,832

U.S. Pat. No. 5,427,910

U.S. Pat. No. 5,523,207

U.S. Pat. No. 4,683,195

U.S. Pat. No. 4,683,202

U.S. Pat. No. 4,683,194

U.S. Pat. No. 5,580,729

Asgeirsson K S, Jonasson J G, Tryggvad L, Olafsd K, Sigurgeirsd J R, Ingvarsson S, Ogmundsd H M. (2000). Eur. J. Cancer, 36(9), 1098-1106.

Atkin N B, Fox M F (1990). 5q deletion. The sole chromosome change in a carcinoma of the bladder. Cancer Genet Cytogenet 46:129-31.

Bohm M, Kirch H, Otto T, Rubben H, Wieland 1 (1997). Deletion analysis at the DEL-27, APC and MTS1 loci in bladder cancer: LOH at the DEL-27 locus on 5p13-12 is a prognostic marker of tumor progression. Int J Cancer 74:291-5.

Brownlee K A. (2^{nd} ed). (1965). Statistical Theory and Methodology in Science and Engineering. John Wiley & Sons, Inc: New York.

Cairns P, Tokino K, Eby Y and Sidransky D. (1994). Cancer Res., 54, 1422-1424

Carter B S, Ewing C M, Ward W S, Treiger B F, Aalders T W, Schalken J A, Epstein J I and Isaacs W B. (1990). Proc Natl Acad Sci USA, 87, 8751-8755.

Chang W Y H, Cairns P, Schoenberg M P, Polascik T J and Sidransky D. (1995). Cancer Res., 55, 3246-3249

Chaturvedi V, Li L, Hodges S, Johnston D, Ro J Y, Logothetis C, von Eschenbach A C, Batsakis J G, Czemiak B (1997). Superimposed histologic and genetic mapping of chromosome 17 alterations in human urinary bladder neoplasia. Oncogene 14:2059-70.

Clinical Correlations, pp. 345-359, Pihiladelphia: Lippincott-Raven, 1995.

Collins, Nat. Genet. 9:347-350, 1995.

Collins, Nat. Genet. 1:3-6, 1992.

Cordon-Cardo, et al. Semin. Surg. Oncol. 13:319-327, 1997.

Cropp et al., Cancer Res., 54, 2548-2551, 1994.

Czemiak B and Herz F. (1995). Diagnostic Cytology of the Urinary Tract with Histopathologic and Clinical Correlations, Koss LG (ed). Lippincott-Raven: Philadelphia, pp. 345-359

Czerniak B, Chaturvedi V, Li L, Hodges S, Johnston D, Ro J Y, Luthra R, Logothetis C, von Eschenbach A C, Grossman B, Benedict W F, Batsakis J G (1999). Superimposed histologic and genetic mapping of chromosome 9 in progression of human urinary bladder neoplasia: implications for a genetic model of multistep urothelial carcinogenesis and early detection of urinary bladder cancer. Oncogene 18:1185-1196.

Czemiak B, Li L, Chaturvedi V, Ro J Y, Johnston D A, Hodges S, Benedict W F (2000). Genetic modeling of human urinary bladder carcinogenesis. Genes Chromosomes Cancer 27:392-402.

Czerniak B. Cohen G L, Etkind P, Deitch D, Simmons H, Herz F and Koss L G. (1992). Hum. Pathol., 23, 1199-1204

Czerniak, et al., Oncogene, 18:1185-1196, 1999.

Dalbagni G, Presti J, Reuter V, Fair W R, Cordon-Cardo C (1993). Genetic alterations in bladder cancer. Lancet 342: 469-71.

Devilee P, Van Vliet M, Van Sloun P, Kuipers-Dijkshoom N, Hermans J, Pearson P L, Cornelisse C J. (1991). Oncogene, 6, 1705-1711.

Dib, et al., Nature, 380:152-154, 1996.

Dutrillaux B, Gerbault-Seureau M, Zafrani B. (1990). Cancer Genet Cytogenet., 49, 203-217

Effenhauser, et al Anal. Chem., 66:2949-2953, 1994.

Fleming et al. (1997) AJCC Cancer Staging Manual. 5th ed. American Joint Committee on Cancer. Lippincott-Raven Publishers: Philadelphia, Pa.

Fodor et al., Nature, 364:555-556, 1993

Freifelder, Physical Biochemistry Applications to Biochemistry and Molecular Biology, 2nd ed. Wm. Freeman and Co., New York, N.Y., 1982.

Gazdar A F, Czerniak B. (2001) Filling the void-urinary markers for bladder cancer risk and diagnosis. J Natl Cancer Inst; 93:413-414.

Gibas Z, Prout G R, Pontes J E, Connolly J G, Sandberg A A (1986). A possible specific chromosome change in transitional cell carcinoma of the bladder. Cancer Genet Cytogenet 19:229-38.

Gorgoulis V G, Mariatos G, Manolis E N, Zacharatos P, Kotsinas A, Liloglou T, Vogiatzi T, Tsagkaraki A, Kokotas S, Tsoli E, Alchanatis M, Sfikakis P P, Asimacopoulos P J, Field J K, Kittas C (2000). Allelic imbalance at the 5q14 locus is associated with decreased apoptotic rate in non-small cell lung carcinomas (NSCLCs). Possible synergistic effect with p53 gene alterations on apoptosis. Lung Cancer 28:211-24.

Greenlee R T, Murray T, Bolden S, Wingo P A (2000). Cancer Statistics, 2000. CA Cancer J Clin 50:7-33.

Gyapay G, Morissette J, Vignal A, Dib C, Fizames C, Millasseau P, Marc S, Bernardi G, Lathrop M and Weissenbach J. (1994). Nat Genet., 7, 246-339.

Habuchi, et al. Int. J. Cancer. 53:579-584, 1993.

Hacia, et al., Nat Genet 4:441-7 1996

Harrison et al., Science, 261:895-897, 1993

Hartigan J A. (1975) Clustering Algorithms. John Wiley & Sons, Inc.: New York.

Hejlik D P, Kottickal L V, Liang H, Fairman J, Davis T, Janecki T, Sexton D, Perry W 3^{rd}, Tavtigian S V, Teng D H, Nagarajan L (1997). Localization of SMAD5 and its evaluation as a candidate myeloid tumor suppressor. Cancer Res 57:3779-83.

Holmstrom, et al., Anal. Biochem. 209:278-283 (1993)

Hughes T R, Roberts C J, Dai H, Jones A R, Meyer M R, Slade D, Burchard J, Dow S, Ward T R, Kidd M J, Friend S H and Marton M J. (2000). Nat Genet., 25, 333-337

Jacobson, et al., Anal. Chem., 66:1107-1113, 1994

Johansson, et al. Genes Chromosomes Cancer. 16:155-163, 1996.

Kamb, et al., Science, 264, 436-440, 1994.

Kinzler, et al. Cell. 87:159-170, 1996.

Klingelhutz A J, Wu S Q, Reznikoff C A (1991). Nonrandom chromosome losses in tumorigenic revertants of hybrids between isogeneic immortal and neoplastic human uroepithelial cells. Som Cell Mol Genet 17:551-65.

Knobloch R, Bugert P, Jauch A, Kalble T, Kovacs G (2000). Allelic changes at multiple regions of chromosome 5 are associated with progression of urinary bladder cancer. J Pathol 190:163-8.

Koo S H, Kwon K C, Ihm C H, Jeon Y M, Park J W, Sul C K (1999). Detection of genetic alterations in bladder tumors by comparative genomic hybridization and cytogenetic analysis. Cancer Genet Cytogenet 110:87-93.

Koss L G and Czerniak B. (1991). N. Engl. J. Med., 324, 125-126

Koss L G, Nakanishi I and Freed S Z. (1977). Urology, 9, 442-455

Koss L G, Tiamson E M Robbins M A (1974). Mapping cancerous and precancerous bladder changes. A study of the urothelium in ten surgically removed bladders. JAMA 227: 281-286.

Koss L G. (1979). Hum. Pathol., 10, 533-548

Koss L G. (ed). (1995). Diagnostic Cytology of the Urinary Tract with Histopathologic and Clinical Correlations. Lippincott-Raven: Philadelphia.

Larsson C, Byrstrom C, Skoog L, Rotstein S, Nordenskjold M. (1990). Genes Chromosome Cancer, 2, 191-197

Lininger R A, Park W S, Man Y G, Pham T, Macgrogan G, Zhuang Z and Tavassoli F A. (1998). Human Pathology, 29(10), 1113-1118.

Liu, et al., Oncogene, 10, 1061-1067, 1995.

Lucito R, West J, Reiner A, Alexander J, Esposito D, Mishra B, Powers S, Norton L, Wigler M (2000). Detecting Gene Copy Number Fluctuations in Tumor Cells By Microarray Analysis of Genomic Representations. Genome Research 10:1726-1736.

Luft F, Gebert J, Schneider A, Melsheimer P, von Knebel Doeberitz M (1999).

Frequent allelic imbalance of tumor suppressor gene loci in cervical dysplasia. Int J Gyn Pathol 18:374-80.

Lynch, C. F., and Cohen, M. B. Urinary System. Cancer(suppl). 75:316-329,

Maniatis T, Fritsch E F and Sambrook J. (1989). Molecular cloning: A laboratory manual. Cold Spring Harbour Laboratory: Cold Spring Harbour, New York.

Manz, et al., ,J. Chromatogr., 593:253-258, 1992.

Mao L, Schoenberg M P, Scicchitano M, Erozan Y S, Merlo A, Schwab D and Sidransky D. (1996). Science, 271, 659-662

Mao, et al. Science. 271:659-662, 1996.

Mostofi F K, editor (1999). Histological Typing of Urinary Bladder Tumors. Springer: New York.

Mullis et al., Cold Spring Harbor Symp. Quant. Biol., 51:263 (1987);

Newton, et al. Nucl. Acids Res. 21:1155-1162 (1993)

Nozawa H, Oda E, Ueda S, Tamura G, Maesawa C, Muto T, Taniguchi T, Tanaka N (1998). Functionally inactivating point mutation in the tumor-suppressor IRF-1 gene identified in human gastric cancer. Int J Cancer 77:522-7.

Onuki N, Wistuba I I, Travis W D, Virmani A K, Yashima K, Brambilla E, Hasleton P, Gazdar A F (1999). Genetic changes in the spectrum of neuroendocrine lung tumors. Cancer 85:600-7.

Ott J. Analysis of Human Genetic Linkage. (1991). Johns Hopkins University Press: Baltimore.

Pan Y, Matsuyama H, Wang N, Yoshihiro S, Haggarth L, Li C, Tribukait B, Ekman P, and Bergerheim US. (1998). Prostate, 36, 31-38.

Parker, et al. Cancer J. Clin. 47:5-27, 1997.

Pease et al., Proc. Natl. Acad. Sci. USA, 91:5022-5026, 1994.

Pollack J R, Perou C M, Alizadet A A, Eisen M B, Pergamenschikov A, Williams C F, Jeffrey S S, Botstein D, Brown P O (1999) Genome-wide analysis of DNA copy number changes using cDNA microarrays. Nat Genet 20:41-46.

Rasmussen, et al., Anal. Biochem. 198:138-142 (1991)

Rodgers C S, Hill S M, Hulten M. (1984). Cancer Genet Cytogenet., 13, 95-119.

Rosin M P, Cairns P, Epstein J I, Schoenberg M P, Sidransky D (1995). Partial allelotype of carcinoma in situ of the human bladder. Cancer Res 55:5213-6.

Ross, et al. Semin. Oncol. 23:536-545, 1996.

Running, et al., BioTechniques 8:276-277 (1990)

Sambrook et al., “Molecular Cloning,” A Laboratory Manual, 2d Ed., Cold Spring Harbor Laboratory Press, New York, 13.7-13.9:1989.

Sandberg, et al. J. Urol. 951:545-560, 1994.

Saran K K, Gould D, Godec C J, Verma R S (1996). Genetics of bladder cancer. J Mol Med 74:441-5.

Sato M, Mori Y, Sakurada A, Fukushige S, Ishikawa Y, Tsuchiya E, Saito Y, Nukiwa T, Fujimura S and Hori A. (1998), Genes Chromosomes Cancer, 22, 1-8.

Sato T, Tanigami A, Yamakawa K, Akiyama F, Kasumi F, Saka O G, Nakamura Y. (1990). Cancer Res., 50, 7184-7189.

Shoemaker, et al, Nat Genet, 4:450-6, 1996

Sidransky, et al. Urol. Clin. N. Am. 1: 629-639, 1992.

Simon R, Burger H, Brinkschmidt C, Bocker W, Hertle L, Terpe H J (1998). Chromosomal aberrations associated with invasion in papillary superficial bladder cancer. J Pathol 185:345-51.

Simoneau A R, Jones P A (1994). Bladder cancer: the molecular progression to invasive disease. World J Urol 12:89-95.

Sobin L H, Wittekind C, editors (1997). TNM classification of malignant tumors. 5th ed. New York: Wiley-Liss.

Spruck C H, Ohneseit P F, Gonzalez-Zulueta M, Esrig D, Miyao N, Tsai Y C, Lerner S P, Schmutte C, Yang A S, Cote R, (1994). Two molecular pathways to transitional cell carcinoma of the bladder. Cancer Res 54:784-8.

Steiner G, Schoenberg M P, Linn J F, Mao L, Sidransky D. (1997). Nature Med., 3, 621-624

Swift et al., Br. J. Cancer., 71:995-998, 1995.

Takahashi T, Habuchi T, Kakehi Y, Mitsumori K, Akao T, Terachi T, Yoshida O (1998). Clonal and chronological genetic analysis of multifocal cancers of the bladder and upper urinary tract. Cancer Res 58:5835-41.

Tsuda et al., Anal. Chem., 62:2149-2152, 1990.

Tsuda H, Zhang W, Shimosato Y, Yokota J, Terada M, Sugimura T, Miyamura T and Hirohashi S. (1990). Proc Natl Acad Sci USA, 87, 6791-6794.

Voorter C, Joos S, Bringuier P P, Vallinga M, Poddighe P, Schalken J, du Manoir S, Ramaekers F, Lichter P, Hopman A (1995). Detection of chromosomal imbalances in transitional cell carcinoma of the bladder by comparative genomic hybridization. Am J Pathol 146:1341-54.

Whitmore S A, Crawford J, Apostolou S, Eyre H, Baker E, Lower K M, Settasatian C, Goldup S, Seshadri R, Gibson R A, Mathew C G, Cleton-Jansen A M, Savoia A, Pronk J C, Auerbach A D, Doggett N A, Sutherland G R and Callen D F. (1998). Genomics, 50, 1-8

What is claimed is:

1. A method of detecting a cell with a neoplastic or preneoplastic phenotype, comprising testing a sample comprising said cell for the presence of LOH (loss of heterozygosity) at one or more loci on one or more chromosomes, wherein said chromosomes are selected from a group consisting of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22, wherein an LOH at said one or more of loci is indicative of a neoplastic or preneoplastic phenotype.

2. The method of claim 1, wherein said cells are obtained from voided urine.

3. The method of claim 1, wherein said cells are obtained from branchial lavage.

4. The method of claim 1, wherein said testing step comprises FISH.

5. The method of claim 1, wherein said testing step comprises the use of a DNA array.

6. The method of claim 5, wherein said testing step comprises the use of a DNA chip.

7. The method of claim 1, wherein said testing step comprises PCR amplification.

8. The method of claim 1, wherein said testing step comprises Southern blotting.

9. The method of claim 1, wherein the neoplastic or preneoplastic phenotype is found in the brain, liver, spleen, lymph node, small intestine, blood cell, pancreatic, colon, stomach, cervix, breast, endometrium, prostate, testicle, ovary, skin, head and neck, esophagus, bone marrow cancer, lung cancer, larynx, oral tissue, kidney and esophagus, bladder, urothelial tissue, or cervix.

10. The method of claim 1, wherein said loci on chromosome 1 are selected from a group consisting of D1S243, D1S1608, D1S548, D1S198, D1S221 and APOA2.

11. The method of claim 1, wherein said loci on chromosome 2 are selected from a group consisting of TPO, D2S1240, D2S378, D2S114, D2S294 and D2S159.

12. The method of claim 1, wherein said loci on chromosome 3 are selected from a group consisting of D3S1298, D3S1278, D3S1303, D3S1541, ACPP, D3S1512, D3S1246, D3S1754, D3S1262 and D3S1661.

13. The method of claim 1, wherein said loci on chromosome 4 are selected from a group consisting of D4S405, D4S828, D4S1548, D4S1597, D4S1607 and D4S408.

14. The method of claim 1, wherein said loci on chromosome 5 are selected from a group consisting of D5S428, APCII, D5S346, D5S421, MCC, D5S659, D5S404, D5S2055, D5S818, IRF1, CFS1R and D5S1465.

15. The method of claim 1, wherein said loci on chromosome 6 are selected from a group consisting of EDN1, D6S251, D6S262, D6S290 and D6S1027.

16. The method of claim 1, wherein said loci on chromosome 7 are selected from a group consisting of D7S526.

17. The method of claim 1, wherein said loci on chromosome 8 are selected from a group consisting of D8S136, D8S133, D8S137, D8S259, ANKI, D8S285 and D8S553.

18. The method of claim 1, wherein said loci on chromosome 9 are selected from a group consisting of D9S286, D9S156, D9S304, D9S273, D9S166, D9S252, D9S287, D9S180 and D9S66.

19. The method of claim 1, wherein said loci on chromosome 10 are selected from a group consisting of D10S1214, D10S213, D10S606, D10S215, D10S1242, D10S190 and D10S217.

20. The method of claim 1, wherein said loci on chromosome 11 are selected from a group consisting of D11S922, D11S569, D11S2368, D11S1301, D11S937, D11S931, D11S897, D11S924, D11S1284, D11S933 and D11S91.

21. The method of claim 1, wherein said loci on chromosome 12 are selected from a group consisting of D12S397.

22. The method of claim 1, wherein said loci on chromosome 13 are selected from a group consisting of D13S221, D13S171, D13S291, RB1, RB1.2, D13S164, D13S268, D13S271 and D13S154.

23. The method of claim 1, wherein said loci on chromosome 14 are selected from a group consisting of D14S290 and D14S68.

24. The method of claim 1, wherein said loci on chromosome 15 are selected from a group consisting of D15S207 and D15 S107.

25. The method of claim 1, wherein said loci on chromosome 16 are selected from a group consisting of D16S513, D16S500, D16S541, D16S415, D16S512, D16S505 and D16S520.

26. The method of claim 1, wherein said loci on chromosome 17 are selected from a group consisting of D17S578, D17S849, TP53, D17S960, D17S786, D17S799, D17S947, D17S579, D17S933, D17S932, D17S934, D17S943, D17S807 and D17S784.

27. The method of claim 1, wherein said loci on chromosome 18 are selected from a group consisting of D18S452, D18S66 and D18S68.

28. The method of claim 1, wherein said loci on chromosome 19 are selected from a group consisting of D19S406, D19S714 and D19S225.

29. The method of claim 1, wherein said loci on chromosome 21 is D21 S212.

30. The method of claim 1, wherein said loci on chromosome 22 are selected from a group consisting of D22S264, D22S446, D22S280 and D22S423.

31. A method of detecting urothelial neoplasia comprising the step of testing one or more samples from an individual for the presence of LOH at one or more loci on one or more chromosomes, wherein said chromosomes are selected from a group consisting of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22, wherein the presence of LOH at one or more of said loci is indicative of the presence of bladder cancer in said individual.

32. The method of claim 31, wherein said urothelial neoplasia comprises the progression of the neoplastic state from preneoplastic conditions to invasive cancer.

33. The method of claim 31, wherein said samples are obtained from voided urine.

34. The method of claim 31, wherein said testing step comprises FISH.

35. The method of claim 31, wherein said testing step comprises the use of a DNA array.

36. The method of claim 35, wherein said testing step comprises the use of a DNA chip.

37. The method of claim 31, wherein said testing step comprises PCR.

38. The method of claim 31, wherein said testing step comprises Southern blotting.

39. The method of claim 31, wherein said loci on chromosome 1 are selected from a group consisting of D1S243, D1S1608, D1S548, D1S198, D1S221 and APOA2.

40. The method of claim 31, wherein said loci on chromosome 2 are selected from a group consisting of TPO, D2S1240, D2S378, D2S114, D2S294 and D2S159.

41. The method of claim 31, wherein said loci on chromosome 3 are selected from a group consisting of D3S1298, D3S1278, D3S1303, D3S1541, ACPP, D3S1512, D3S1246, D3S1754, D3S1262 and D3S1661.

42. The method of claim 31, wherein said loci on chromosome 4 are selected from a group consisting of D4S405, D4S828, D4S1548, D4S1597, D4S1607 and D4S408.

43. The method of claim 31, wherein said loci on chromosome 5 are selected from a group consisting of D5S428, APCII, D5S346, D5S421, MCC, D5S659, D5S404, D5S2055, D5S818, IRF1, CFS1R and D5S1465.

44. The method of claim 31, wherein said loci on chromosome 6 are selected from a group consisting of EDN1, D6S251, D6S262, D6S290 and D6S1027.

45. The method of claim 31, wherein said loci on chromosome 7 are selected from a group consisting of D7S526.

46. The method of claim 31, wherein said loci on chromosome 8 are selected from a group consisting of D8S136, D8S133, D8S137, D8S259, ANKI, D8S285 and D8S553.

47. The method of claim 31, wherein said loci on chromosome 9 are selected from a group consisting of D9S286, D9S156, D9S304, D9S273, D9S166, D9S252, D9S287, D9S180 and D9S66.

48. The method of claim 31, wherein said loci on chromosome 10 are selected from a group consisting of D10S1214, D10S213, D10S606, D10S215, D10S1242, D10S190 and D10S217.

49. The method of claim 31, wherein said loci on chromosome 11 are selected from a group consisting of D11S922, D11S569, D11S2368, D11S1301, D11S937, D11S931, D11S897, D11S924, D11S1284, D11S933 and D11S910.

50. The method of claim 31, wherein said loci on chromosome 12 are selected from a group consisting of D12S397.

51. The method of claim 31 wherein said loci on chromosome 13 are selected from a group consisting of D13S221, D13S171, D13S291, RB1, RB1.2, D13S164, D13S268, D13S271 and D13S154.

52. The method of claim 31, wherein said loci on chromosome 14 are selected from a group consisting of D14S290 and D14S68.

53. The method of claim 31, wherein said loci on chromosome 15 are selected from a group consisting of D15S207 and D15S107.

54. The method of claim 31, wherein said loci on chromosome 16 are selected from a group consisting of D16S513, D16S500, D16S541, D16S415, D16S512, D16S505 and D16S520.

55. The method of claim 31, wherein said loci on chromosome 17 are selected from a group consisting of D17S578, D17S849, TP53, D17S960, D17S786, D17S799, D17S947, D17S579, D17S933, D17S932, D17S934, D17S943, D17S807 and D17S784.

56. The method of claim 31, wherein said loci on chromosome 18 are selected from a group consisting of D18S452, D18S66 and D18S68.

57. The method of claim 31, wherein said loci on chromosome 21 is D21S212.

58. The method of claim 31, wherein said loci on chromosome 19 are selected from a group consisting of D19S406, D19S714 and D19S225.

59. The method of claim 31, wherein said loci on chromosome 22 are selected from a group consisting of D22S264, D22S446, D22S280 and D22S423.

60. A DNA array for use in the detection of a neoplasia or preneoplastic phenotype, said DNA array comprising DNA probes, said DNA probes selected to detect LOH at one or more loci on chromosomes, wherein said chromosomes are selected from a group consisting of chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21 and chromosome 22, wherein an LOH at one or more of said loci is indicative of a neoplastic or preneoplastic phenotype.

61. The method of claim 60, wherein the neoplastia or preneoplastic phenotype is found in the brain, liver, spleen, lymph node, small intestine, blood cell, pancreatic, colon, stomach, cervix, breast, endometrium, prostate, testicle, ovary, skin, head and neck, esophagus, bone marrow cancer, lung cancer, larynx, oral tissue, kidney and esophagus, bladder, urothelial tissue, or cervix.

62. The DNA array of claim 60, wherein said neoplasia is urothelial neoplasia

63. The method of claim 60, wherein said loci on chromosome 1 are selected from a group consisting of D1S243, D1S1608, D1S548, D1S198, D1S221 and APOA2.

64. The method of claim 60, wherein said loci on chromosome 2 are selected from a group consisting of TPO, D2S1240, D2S378, D2S114, D2S294 and D2S159.

65. The method of claim 60, wherein said loci on chromosome 3 are selected from a group consisting of D3S1298, D3S1278, D3S1303, D3S1541, ACPP, D3 S1512, D3 S1246, D3S1754, D3S1262 and D3S1661.

66. The method of claim 60, wherein said loci on chromosome 4 are selected from a group consisting of D4S405, D4S828, D4S1548, D4S1597, D4S1607 and D4S408.

67. The method of claim 60, wherein said loci on chromosome 5 are selected from a group consisting of D5S428, APCII, D5S346, D5S421, MCC, D5S659, D5S404, D5S2055, D5S818, IRF1, CFS1R and D5S1465.

68. The method of claim 60, wherein said loci on chromosome 6 are selected from a group consisting of EDN1, D6S251, D6S262, D6S290 and D6S1027.

69. The method of claim 60, wherein said loci on chromosome 7 are selected from a group consisting of D7S526.

70. The method of claim 60, wherein said loci on chromosome 8 are selected from a group consisting of D8S136, D8S133, D8S137, D8S259, ANKI, D8S285 and D8S553.

71. The method of claim 60, wherein said loci on chromosome 9 are selected from a group consisting of D9S286, D9S156, D9S304, D9S273, D9S166, D9S252, D9S287, D9S180 and D9S66.

72. The method of claim 60, wherein said loci on chromosome 10 are selected from a group consisting of D10S1214, D10S213, D10S606, D10S215, D110S1242, D10S190 and D110S217.

73. The method of claim 60, wherein said loci on chromosome 11 are selected from a group consisting of D11S922, D11S569, D11S2368, D11S1301, D11S937, D11S931, D11S897, D11S924, D11S1284, D11S933 and D11S910.

74. The method of claim 60, wherein said loci on chromosome 12 are selected from a group consisting of D12S397.

75. The method of claim 60, wherein said loci on chromosome 13 are selected from a group consisting of D13S221, D13S171, D13S291, RB1, RB1.2, D13S164, D13S268, D13S271 and D13S154.

76. The method of claim 60, wherein said loci on chromosome 14 are selected from a group consisting of D14S290 and D14S68.

77. The method of claim 60, wherein said loci on chromosome 15 are selected from a group consisting of D15S207 and D15S107.

78. The method of claim 60, wherein said loci on chromosome 16 are selected from a group consisting of D16S513, D16S500, D16S541, D16S415, D16S512, D16S505 and D16S520.

79. The method of claim 60, wherein said loci on chromosome 17 are selected from a group consisting of D17S578, D17S849, TP53, D17S960, D17S786, D17S799, D17S947, D17S579, D17S933, D17S932, D17S934, D17S943, D17S807 and D17S784.

80. The method of claim 60, wherein said loci on chromosome 18 are selected from a group consisting of D18S452, D18S66 and D18S68.

81. The method of claim 60, wherein said loci on chromosome 19 are selected from a group consisting of D19S406, D19S714 and D19S225.

82. The method of claim 60, wherein said loci on chromosome 21 is D21S212.

83. The method of claim 60, wherein said loci on chromosome 22 are selected from a group consisting of D22S264, D22S446, D22S280 and D22S423.

84. A method of detecting occult preclinical or premicroscopic stages of urothelial neoplasia, comprising:
a) obtaining a urine sample;
b) isolating bladder cells from said sample; and
c) testing said bladder cells for allelic loss at one or more loci associated with the development of urothelial neoplasia;
wherein said loci are selected from the group consisting of D1S243, D1S1608, D1S548, D1S198, D1S221, APOA2, TPO, D2S1240, D2S378, D2S114, D2S294, D2S159, D3S1298, D3S1278, D3S1303, D3S1541, ACPP, D3S1512, D3S1246, D3S1754, D3S1262 and D3S1661 D4S405, D4S828, D4S1548, D4S1597, D4S1607, D4S408, D5S428, APCII, D5S346, D5S421, MCC, D5S659, D5S404, D5S2055, D5S818, IRF1, CFS1R, D5S1465, EDN1, D6S251, D6S262, D6S290, D6S1027, D7S526, D8S136, D8S133, D8S137, D8S259, ANKI, D8S285, D8S553, D9S286, D9S156, D9S304, D9S273, D9S166, D9S252, D9S287, D9S180, D9S66, D10S1214, D10S213, D10S606, D10S215, D10S1242, D10S190, D10S217, D11S922, D11S569, D11S2368, D11S1301, D11S937, D11S931, D11S897, D11S924, D11S1284, D11S933, D11S910, D12S397, D13S221, D13S171, D13S291, RB1, RB1.2, D13S164, D13S268, D13S271, D13S154, D14S290, D14S68, D15S207, D15S107, D16S513, D16S500, D16S541, D16S415, D16S512, D16S505, D16S520, D17S578, D17S849, TP53, D17S960, D17S786, D17S799, D17S947, D17S579, D17S933, D17S932, D17S934, D17S943, D17S807, D17S784, D18S452, D18S66, D18S68, D19S406, D19S714, D19S225, D21S212, D22S264, D22S446, D22S280 and D22S423.

85. The method of claim 84, wherein said testing step comprises FISH.

86. The method of claim 84, wherein said testing step comprises the use of a DNA array.

87. The method of claim 86, wherein said testing step comprises the use of a DNA chip.

88. The method of claim 84, wherein said testing step comprises PCR.

89. The method of claim 84, wherein said testing step comprises Southern blotting.

90. A method of detecting urothelial neoplasia, comprising:
a) obtaining a urine sample;
b) isolating bladder cells from said sample; and
c) testing said bladder cells for allelic loss at one or more loci associated with the development of urothelial neoplasia;
wherein said loci are selected from the group consisting D1S243, D1S1608, D1S548, D1S198, D1S221, APOA2, TPO, D2S1240, D2S378, D2S114, D2S294, D2S159, D3S1298, D3S1278, D3S1303, D3S1541, ACPP, D3S1512, D3S1246, D3S1754, D3S1262 and D3S1661 D4S405, D4S828, D4S1548, D4S1597, D4S1607, D4S408, D5S428, APCII, D5S346, D5S421, MCC, D5S659, D5S404, D5S2055, D5S818, IRF1, CFS1R, D5S1465, EDN1, D6S251, D6S262, D6S290, D6S1027, D7S526, D8S136, D8S133, D8S137, D8S259, ANKI, D8S285, D8S553, D9S286, D9S156, D9S304, D9S273, D9S166, D9S252, D9S287, D9S180, D9S66, D10S1214, D10S213, D10S606, D10S215, D10S1242, D10S190, D10S217, D11S922, D11S569, D11S2368, D11S1301, D11S937, D11S931, D11S897, D11S924, D11S1284, D11S933, D11S910, D12S397, D13S221, D13S171, D13S291, RB1, RB1.2, D13S164, D13S268, D13S271, D13S154, D14S290, D14S68, D15S207, D15S107, D16S513, D16S500, D16S541, D16S415, D16S512, D16S505, D16S520, D17S578, D17S849, TP53, D17S960, D17S786, D17S799, D17S947, D17S579, D17S933, D17S932, D17S934, D17S943, D17S807, D17S784, D18S452, D18S66, D18S68, D19S406, D19S714, D19S225, D21S212, D22S264, D22S446, D22S280 and D22S423.

91. The method of claim 90, wherein said testing step comprises FISH.

92. The method of claim 90, wherein said testing step comprises the use of a DNA array.

93. The method of claim 90, wherein said testing step comprises the use of a DNA chip.

94. The method of claim 90, wherein said testing step comprises PCR.

95. The method of claim 90, wherein said testing step comprises Southern blotting.


SEQUENCE LISTING (20 sequences)

SEQ ID NO: 1 - DNA, 19 bp, Artificial Sequence
  Description of Artificial Sequence  Synthetic Primer
gaagaaagaggaggggctg

SEQ ID NO: 2 - DNA, 20 bp, Artificial Sequence
  Description of Artificial Sequence  Synthetic Primer
gcgctacctgattccaattc

SEQ ID NO: 3 - DNA, 20 bp, Artificial Sequence
  Description of Artificial Sequence  Synthetic Primer
ggaaattggaaactggaagc

SEQ ID NO: 4 - DNA, 19 bp, Artificial Sequence
  Description of Artificial Sequence  Synthetic Primer
tctgagctttggaagctct

SEQ ID NO: 5 - DNA, 18 bp, Artificial Sequence
  Description of Artificial Sequence  Synthetic Primer
ttctttctgccctctgca

SEQ ID NO: 6 - DNA, 20 bp, Artificial Sequence
  Description of Artificial Sequence  Synthetic Primer
gcagttgtggccctgtagga

SEQ ID NO: 7 - DNA, 20 bp, Artificial Sequence
  Description of Artificial Sequence  Synthetic Primer
ccagaagcaatccaggcgcg

SEQ ID NO: 8 - DNA, 19 bp, Artificial Sequence
  Description of Artificial Sequence  Synthetic Primer
aatgcacacctcgccaacg

SEQ ID NO: 9 - DNA, 20 bp, Artificial Sequence
  Description of Artificial Sequence  Synthetic Primer
tgagtttaacctgaaggtgg

SEQ ID NO: 10 - DNA, 19 bp, Artificial Sequence
  Description of Artificial Sequence  Synthetic Primer
gggtgggaaattgggtaag

SEQ ID NO: 11 - DNA, 20 bp, Artificial Sequence
  Description of Artificial Sequence  Synthetic Primer
ttcctcttcctgcagtactc

SEQ ID NO: 12 - DNA, 21 bp, Artificial Sequence
  Description of Artificial Sequence  Synthetic Primer
accctgggcaaccagccctgt

SEQ ID NO: 13 - DNA, 21 bp, Artificial Sequence
  Description of Artificial Sequence  Synthetic Primer
acagggctggttgcccagggt

SEQ ID NO: 14 - DNA, 19 bp, Artificial Sequence
  Description of Artificial Sequence  Synthetic Primer
agttgcaaaccagacctat

SEQ ID NO: 15 - DNA, 20 bp, Artificial Sequence
  Description of Artificial Sequence  Synthetic Primer
gtgttgtctcctaggttggc

SEQ ID NO: 16 - DNA, 20 bp, Artificial Sequence
  Description of Artificial Sequence  Synthetic Primer
gtcagaggcaagcagaggct

SEQ ID NO: 17 - DNA, 20 bp, Artificial Sequence
  Description of Artificial Sequence  Synthetic Primer
tatcctgagtagtggtaatc

SEQ ID NO: 18 - DNA, 20 bp, Artificial Sequence
  Description of Artificial Sequence  Synthetic Primer
aagtgaatctgaggcataac

SEQ ID NO: 19 - DNA, 21 bp, Artificial Sequence
  Description of Artificial Sequence  Synthetic Primer
gcagttatgcctcagattcac

SEQ ID NO: 20 - DNA, 21 bp, Artificial Sequence
  Description of Artificial Sequence  Synthetic Primer
aagacttagtacctgaagggt